(19) United States
(12) Patent Application Publication
(10) Pub. No.: US 2013/0273029 A1
(43) Pub. Date: Oct. 17, 2013

(54) ANTIBODIES FOR EPIDERMAL GROWTH FACTOR RECEPTOR 3 (HER3) DIRECTED TO DOMAIN II OF HER3

(71) Applicant: NOVARTIS AG, Basel (CH)

(72) Inventors: Winfried ELIS, Munchen-Pasing (DE); Seth ETTENBERG, Melrose, MA (US); Andrew Paul GARNER, Arlington, MA (US); Christian Carsten Silvester KUNZ, Munchen (DE); Tobias SEITZ, Munich (DE)

(21) Appl. No.: 13/693,339

(22) Filed: Dec. 4, 2012

Related U.S. Application Data
(60) Provisional application No. 61/566,905, filed on Dec. 5, 2011.

(51) Int. Cl. C07K 16/28; A61K 39/395; A61K 45/06

(52) U.S. Cl. CPC C07K 16/2863; A61K 45/06; A61K 39/3955
USPC 424/133.1; 530/387.9; 530/387.3; 424/139.1

(57) ABSTRACT

The present invention relates to antibodies or fragments thereof that target an epitope of a HER3 receptor residing in domain 2 of the HER3 receptor to block both ligand-dependent and ligand-independent signal transduction and tumor growth; and compositions and methods of use thereof.

### RELATED APPLICATIONS

This application claims priority to U.S. Provisional Application No. 61/566,905 filed on Dec. 5, 2011, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Jun. 17, 2013, is named PAT054913-US-NP_SL.txt and is 381,423 bytes in size.

### FIELD OF THE INVENTION

This invention relates generally to antibodies or fragments thereof that recognize an epitope of HER3 comprising residues within domain 2 resulting in inhibition of both ligand-dependent and ligand-independent signal transduction and tumor growth; and compositions and methods of use of such antibodies or fragments thereof.

### BACKGROUND OF THE INVENTION

The human epidermal growth factor receptor 3 (ErbB3, also known as HER3) is a receptor protein tyrosine kinase and belongs to the epidermal growth factor receptor (EGFR) subfamily of receptor protein tyrosine kinases, which also includes EGFR (HER1, ErbB1), HER2 (ErbB2, Neu), and HER4 (ErbB4) (Plowman et al., (1990) Proc. Natl. Acad. Sci. U.S.A. 87:4905-4909; Kraus et al., (1989) Proc. Natl. Acad. Sci. U.S.A. 86:9193-9197; and Kraus et al., (1993) Proc. Natl. Acad. Sci. U.S.A. 90:2900-2904). Like the prototypical epidermal growth factor receptor, the transmembrane receptor HER3 consists of an extracellular ligand-binding domain (ECD), a dimerization domain within the ECD, a transmembrane domain, an intracellular protein tyrosine kinase-like domain (TKD) and a C-terminal phosphorylation domain. Unlike the other HER family members, the kinase domain of HER3 displays very low intrinsic kinase activity.

The ligands neuregulin 1 (NRG) or neuregulin 2 bind to the extracellular domain of HER3 and activate receptor-mediated signaling pathway by promoting dimerization with other dimerization partners such as HER2. Heterodimerization results in activation and transphosphorylation of HER3's intracellular domain and is a means not only for signal diversification but also signal amplification. In addition, HER3 heterodimerization can also occur in the absence of activating ligands and this is commonly termed ligand-independent HER3 activation. For example, when HER2 is expressed at high levels as a result of gene amplification (e.g. in breast, lung, ovarian or gastric cancer) spontaneous HER2/HER3 dimers can be formed. In this situation the HER2/HER3 is considered the most active ErbB signaling dimer and is therefore highly transforming.

Increased HER3 has been found in several types of cancer such as breast, lung, gastrointestinal and pancreatic cancers. Interestingly, a correlation between the expression of HER2/HER3 and the progression from a non-invasive to an invasive stage has been shown (Alimandi et al., (1995) Oncogene 10:1813-1821; DeFazio et al., (2000) Cancer 87:487-498; Naidu et al., (1988) Br. J. Cancer 78:1385-1390). Accordingly, agents that interfere with HER3 mediated signaling are needed.

### SUMMARY OF THE INVENTION

The invention is based on the discovery of antibodies or fragments thereof that bind to an epitope (linear, non-linear, conformational) of HER3 receptor comprising amino acid residues within domain 2 of HER3. Surprisingly, binding of the antibodies or fragments thereof to an epitope within domain 2 of HER3 blocks both ligand-dependent (e.g. neuregulin) and ligand-independent HER3 signaling pathways.

Accordingly, in one aspect, the invention pertains to an isolated antibody or fragment thereof that recognizes an epitope of a HER3 receptor, wherein the epitope comprises amino acid residues 208-328 within domain 2 of the HER3 receptor, wherein the antibody or fragment thereof recognizes at least amino acid residue 268 within domain 2, and wherein the antibody or fragment thereof blocks both ligand-dependent and ligand-independent signal transduction.

The eptiope is selected from the group consisting of a linear epitope, a non-linear epitope, and a conformational epitope. In one embodiment, the antibody or fragment thereof binds to an inactive state of the HER3 receptor. In one embodiment, HER3 ligand binding to the ligand binding site fails to activate HER3 signal transduction. In one embodiment, a HER3 ligand can concurrently bind to the ligand binding site on the HER3 receptor. In one embodiment, the HER3 ligand is selected from the group consisting of neuregulin 1 (NRG), neuregulin 2, betacellulin, heparin-binding epidermal growth factor, and epiregulin. The antibodies or fragments thereof described herein can bind to amino acid residue 268 (within domain 2). In one embodiment, binding amino acid 268 affects binding in domain 2, thereby blocking antibody or antibody fragment binding. In one embodiment, the antibody or fragment thereof has a characteristic selected from the group consisting of destabilizing HER3 such that it is susceptale to degradation, accelerating down regulation of cell surface HER3, inhibiting dimerization with other HER receptors, and generating an un-natural HER3 dimer that is susceptible to proteolytic degradation or unable to dimerize with other receptor tyrosine kinases. In one embodiment, the binding of the antibody or fragment thereof to the HER3 receptor in the absence of a HER3 ligand reduces ligand-independent formation of a HER2-HER3 protein complex in a cell which expresses HER2 and HER3. In one embodiment, the HER3 receptor fails to dimerize with the HER2 receptor to form a HER2-HER3 protein complex. In one embodiment, the failure to form a HER2-HER3 protein complex prevents activation of signal transduction. In one embodiment, the antibody or fragment thereof inhibits phosphorylation of HER3 as assessed by a HER3 ligand-independent phosphorylation assay. In one embodiment, the HER3 ligand-independent phosphorylation assay uses HER2 amplified cells, wherein the HER2 amplified cells are SK-Br-3 cells and BT-474. In one embodiment, binding of the antibody or fragment thereof to the HER3 receptor in the presence of a HER3 ligand reduces ligand-dependent formation of a HER2-HER3 protein complex in a cell which expresses HER2 and HER3. In one embodiment, the HER3 receptor fails to dimerize with the HER2 receptor in the presence of a HER3 ligand to form a HER2-HER3 protein complex. In one embodiment, the failure to form a HER2-HER3 protein complex prevents activation of signal transduction. In one embodiment, the antibody or fragment thereof inhibits phosphorylation of HER3 as assessed by HER3 ligand-dependent phosphorylation assay. In one embodiment, the HER3 ligand-dependent phosphorylation assay uses stimulated MCF7 cells in the presence of neuregulin (NRG). In one embodiment, the antibody is selected from the group consisting of a monoclonal antibody, a polyclonal antibody, a chimeric antibody, a humanized antibody, and a synthetic antibody.

In another aspect, the invention pertains to an isolated antibody or fragment thereof that recognizes a epitope of a HER3 receptor within domain 2 of the HER3 receptor, wherein the epitope comprises amino acid residues 208-328 within domain 2 of the HER3 receptor, wherein the antibody or fragment thereof recognizes at least amino acid residue 268 within domain 2, and wherein the antibody or fragment thereof has a dissociation (K_{D}) of at least 1×10⁷ M⁻¹, 10⁸ M⁻¹, 10⁹ M⁻¹, 10¹⁰ M⁻¹, 10¹¹ M⁻¹,10¹² M⁻¹, 10¹³ M⁻¹, and wherein the antibody or fragment thereof blocks both ligand-dependent and ligand-independent signal transduction. In one embodiment, the antibody or fragment thereof inhibits phosphorylation of HER3 as measured by an in vitro phosphorylation assay selected from the group consisting of phospho-HER3 and phospho-Akt. In one embodiment, the antibody or fragment thereof binds to the same epitope as an antibody described in Table 1. In one embodiment, the isolated antibody or fragment thereof cross-competes with an antibody described in Table 1. In one embodiment, the fragment of an antibody that selected from the group consisting of; Fab, F(ab₂)′, F(ab)₂′, scFv, VHH, VH, VL, dAbs.

In another aspect, the invention pertains to a pharmaceutical composition comprising an antibody or fragment thereof and a pharmaceutically acceptable carrier, wherein the antibody or fragment thereof binds a HER3 receptor comprising amino acid residues 208-328 within domain 2 of the HER3 receptor, wherein the antibody or fragment thereof recognizes at least amino acid residue 268 within domain 2, and wherein the antibody or fragment thereof blocks both ligand-dependent and ligand-independent signal transduction. In one embodiment, the pharmaceutical composition further comprises an additional therapeutic agent. In one embodiment, the additional therapeutic agent is selected from the group consisting of an HER1 inhibitor, a HER2 inhibitor, a HER3 inhibitor, a HER4 inhibitor, an mTOR inhibitor and a PI3 Kinase inhibitor. In one embodiment, the additional therapeutic agent is a HER1 inhibitor selected from the group consisting of Matuzumab (EMD72000), Erbitux®/Cetuximab, Vectibix®/Panitumumab, mAb 806, Nimotuzumab, Iressa®/Gefitinib, CI-1033 (PD183805), Lapatinib (GW-572016), Tykerb®/Lapatinib Ditosylate, Tarceva®/Erlotinib HCL (OSI-774), PKI-166, and Tovok®; a HER2 inhibitor selected from the group consisting of Pertuzumab, Trastuzumab, MM-111, neratinib, lapatinib or lapatinib ditosylate/Tykerb®; a HER3 inhibitor selected from the group consisting of, MM-121, MM-111, IB4C3, 2DID12 (U3 Pharma AG), AMG888 (Amgen), AV-203 (Aveo), MEHD7945A (Genentech), MOR10703 (Novartis), and small molecules that inhibit HER3; and a HER4 inhibitor. In one embodiment, the additional therapeutic agent is an mTOR inhibitor selected from the group consisting of Temsirolimus/Torisel®, ridaforolimus/Deforolimus, AP23573, MK8669, everolimus/Affinitor®. In one embodiment, the additional therapeutic agent is a PI3 Kinase inhibitor selected from the group consisting of GDC 0941, BEZ235, BMK120 and BYL719.

In another aspect, the invention pertains to a method of treating a cancer comprising selecting a subject having an HER3 expressing cancer, administering to the subject an effective amount of a composition comprising an antibody or fragment thereof disclosed in Table 1, wherein the antibody or fragment thereof recognizes an epitope of a HER3 receptor comprising amino acid residues 208-328 within domain 2 of the HER3 receptor, wherein the antibody or fragment thereof recognizes at least amino acid residue 268 within domain 2, and wherein the antibody or fragment thereof blocks both ligand-dependent and ligand-independent signal transduction. In one embodiment, the subject is a human and the cancer is selected from the group consisting of breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis. In one embodiment, the cancer is breast cancer.

In one aspect, the invention pertains to an antibody or fragment thereof for use in treating a cancer mediated by a HER3 ligand-dependent signal transduction or ligand-independent signal transduction pathway. In one aspect, the invention pertains to an antibody or fragment thereof for use as a medicament. In one aspect, the invention pertains to use of an antibody or fragment thereof for the manufacture of a medicament for the treatment of a cancer mediated by a HER3 ligand-dependent signal transduction or ligand-independent signal transduction pathway selected from the group consisting of breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis.

### BRIEF DESCRIPTION OF FIGURES

FIG. 1: Representative MOR12616 (A) and MOR12925 (B) SET curves obtained with human HER3;

FIG. 2: SK-Br-3 cell binding determination by FACS titration;

FIG. 3: HER3 domain binding ELISA titration curves for MOR12616 (A) and MOR12925 (B);

FIG. 4: HER3 mutant binding ELISA curves binding to (A) human HER3 (WT) and (B) human mutant HER3 (L268A);

FIG. 5: HER3 epitope competition by ELISA determined with (A) 12925 competition; and (B) 12616 competition;

FIG. 6: Inhibition of ligand induced (A) HER3 and (B) Akt phosphorylation;

FIG. 7: Inhibition of ligand independent HER3 and Akt phosphorylation in HER2 amplified cell lines with MOR12925 shown as (A) pHER3 in unstimulated SKBR3 cells, (B) pAKT in unstimulated SKBR3 cells, (C) pHER3 unstimulated BT474 cells, and (D) pAKT in unstimulated BT474 cells;

FIG. 8: Inhibition of (A) ligand dependent and (B, C) ligand independent cell proliferation; and

FIG. 9: Data showing in vivo inhibition of tumor growth in BxPC3 (A) and BT474 (B).

### DETAILED DESCRIPTION OF THE INVENTION

### Definitions

In order that the present invention may be more readily understood, certain terms are first defined. Additional definitions are set forth throughout the detailed description.

The phrase “signal transduction” or “signaling activity” as used herein refers to a biochemical causal relationship generally initiated by a protein-protein interaction such as binding of a growth factor to a receptor, resulting in transmission of a signal from one portion of a cell to another portion of a cell. For HER3, the transmission involves specific phosphorylation of one or more tyrosine, serine, or threonine residues on one or more proteins in the series of reactions causing signal transduction. Penultimate processes typically include nuclear events, resulting in a change in gene expression.

The term “HER3” or “HER3 receptor” also known as “ErbB3” as used herein refers to mammalian HER3 protein and “her3” or “erbB3” refers to mammalian her3 gene. The preferred HER3 protein is human HER3 protein present in the cell membrane of a cell. The human her3 gene is described in U.S. Pat. No. 5,480,968 and Plowman et al., (1990) Proc. Natl. Acad. Sci. USA, 87:4905-4909.

Human HER3 as defined in Accession No. NP_{—}001973 (human), and represented below as SEQ ID NO: 1. All nomenclature is for full length, immature HER3 (amino acids 1-1342). The immature HER3 is cleaved between positions 19 and 20, resulting in the mature HER3 protein (20-1342 amino acids).

(SEQ ID NO: 1)
mrandalqvl gllfslargs evgnsqavcp gtlnglsvtg daenqyqtly klyercevvm
gnleivltgh nadlsflqwi revtgyvlva mnefstlplp nlrvvrgtqv ydgkfaifvm
lnyntnssha lrqlrltqlt eilsggvyie kndklchmdt idwrdivrdr daeivvkdng
rscppchevc kgrcwgpgse dcqtltktic apqcnghcfg pnpnqcchde caggcsgpqd
tdcfacrhfn dsgacvprcp qplyynkltf qlepnphtky qyggvcvasc phnfvvdqts
cvracppdkm evdknglkmc epcgglcpka cegtgsgsrf qtvdssnidg fvnctkilgn
ldflitglng dpwhkipald peklnvfrtv reitgylniq swpphmhnfs vfsnlttigg
rslynrgfsl limknlnvts lgfrslkeis agriyisanr qlcyhhslnw tkvlrgptee
rldikhnrpr rdcvaegkvc dplcssggcw gpgpgqclsc rnysrggvcv thcnflngep
refaheaecf schpecqpme gtatcngsgs dtcaqcahfr dgphcvsscp hgvlgakgpi
ykypdvqnec rpchenctqg ckgpelqdcl gqtlvligkt hltmaltvia glvvifmmlg
gtflywrgrr iqnkramrry lergesiepl dpsekankvl arifketelr klkvlgsgvf
gtvhkgvwip egesikipvc ikviedksgr qsfqavtdhm laigsldhah ivrllglcpg
sslqlvtqyl plgslldhvr qhrgalgpql llnwgvqiak gmyyleehgm vhrnlaarnv
llkspsqvqv adfgvadllp pddkqllyse aktpikwmal esihfgkyth qsdvwsygvt
vwelmtfgae pyaglrlaev pdllekgerl aqpqictidv ymvmvkcwmi denirptfke
laneftrmar dpprylvikr esgpgiapgp ephgltnkkl eevelepeld ldldleaeed
nlatttlgsa lslpvgtlnr prgsqsllsp ssgympmnqg nlgescqesa vsgssercpr
pvslhpmprg clasessegh vtgseaelqe kvsmcrsrsr srsprprgds ayhsqrhsll
tpvtplsppg leeedvngyv mpdthlkgtp ssregtlssv glssvlgtee ededeeyeym
nrrrrhspph pprpssleel gyeymdvgsd lsaslgstqs cplhpvpimp tagttpdedy
eymnrqrdgg gpggdyaamg acpaseqgye emrafqgpgh qaphyhyarl ktlrsleatd
safdnpdywh srlfpkanaq rt

The term “HER3 ligand” as used herein refers to polypeptides which bind and activate HER3. Examples of HER3 ligands include, but are not limited to neuregulin 1 (NRG) and neuregulin 2, betacellulin, heparin-binding epidermal growth factor, and epiregulin. The term includes biologically active fragments and/or variants of a naturally occurring polypeptide.

The “HER2-HER3 protein complex” is a noncovalently associated oligomer containing HER2 receptor and the HER3 receptor. This complex can form when a cell expressing both of these receptors is exposed to a HER3 ligand e.g., NRG or when HER2 is active/overexpressed

The phrase “HER3 activity” or “HER3 activation” as used herein refers to an increase in oligomerization (e.g. an increase in HER3 containing complexes), HER3 phosphorylation, conformational rearrangements (for example those induced by ligands), and HER3 mediated downstream signaling.

The term “stabilization” or “stabilized” used in the context of HER3 refers to an antibody or fragment thereof that directly maintains (locks, tethers, holds, preferentially binds, favors) the inactive state or conformation of HER3 without blocking ligand binding to HER3, such that ligand binding is no longer able to activate HER3.

The term “ligand-dependent signaling” as used herein refers to the activation of HER3 via ligand. HER3 activation is evidenced by increased heterodimerization and/or HER3 phosphorylation such that downstream signaling pathways (e.g. PI3K) are activated. The antibody or fragment thereof can statistically significantly reduce the amount of phosphorylated HER3 in a stimulated cell exposed to an antibody or fragment thereof relative to an untreated (control) cell, as measured using the assays described in the Examples. The cell which expresses HER3 can be a naturally occurring cell line (e.g. MCF7) or can be recombinantly produced by introducing nucleic acids encoding HER3 protein into a host cell. Cell stimulation can occur either via the exogenous addition of an activating HER3 ligand or by the endogenous expression of an activating ligand.

The antibody or fragment thereof which “reduces neregulin-induced HER3 activation in a cell” is one which statistically significantly reduces HER3 tyrosine phosphorylation relative to an untreated (control) cell, as measured using the assays described in the Examples. This can be determined based on HER3 phosphotyrosine levels following exposure of HER3 to NRG and the antibody of interest. The cell which expresses HER3 protein can be a naturally occurring cell or cell line (e.g. MCF7) or can be recombinantly produced.

The term “ligand-independent signaling” as used herein refers to cellular HER3 activity (e.g. phosphorylation) in the absence of a requirement for ligand binding. For example, ligand-independent HER3 activation can be a result of HER2 overexpression or activating mutations in HER3 heterodimer partners such as EGFR and HER2. The antibody or fragment thereof can statistically significantly reduce the amount of phosphorylated HER3 in a cell exposed to an antibody or fragment thereof relative to an untreated (control) cell. The cell which expresses HER3 can be a naturally occurring cell line (e.g. SK-Br-3) or can be recombinantly produced by introducing nucleic acids encoding HER3 protein into a host cell.

The term “blocks” as used herein refers to stopping or preventing an interaction or a process, e.g., stopping ligand-dependent or ligand-independent signaling.

The term “recognize” as used herein refers to an antibody or fragment thereof that finds and interacts (e.g., binds) with its epitope in domain 2 of HER3. For example, an antibody or fragment thereof that interacts with at least one amino acid residue within domain 2 of HER3 (amino acid residues 208-328 of SEQ ID NO: 1). In another example, the antibody or fragment thereof that interacts with at least Lys 268 within domain 2 of HER3.

The phrase “concurrently binds” as used herein refers to a HER3 ligand that can bind to a ligand binding site on the HER3 receptor along with the HER3 antibody or fragment thereof. This means that both the antibody and ligand can bind to the HER3 receptor together. For the sake of illustration only, the HER3 ligand NRG, can bind to the HER3 receptor along with the HER3 antibody. Assay to measure concurrent binding of the ligand and antibody are described in the Examples section.

The term “fails” as used herein refers to an antibody or fragment thereof that does not do a particular event. For example, an antibody or fragment thereof that “fails to activate signal transduction” is one that does not trigger signal transduction.

The term “antibody” as used herein refers to whole antibodies that interact with (e.g., by binding, steric hindrance, stabilizing/destabilizing, spatial distribution) an HER3 epitope and inhibit signal transduction. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The phrase “antibody fragment”, as used herein, refers to one or more portions of an antibody that retain the ability to specifically interact with (e.g., by binding, steric hindrance, stabilizing/destabilizing, spatial distribution) an HER3 epitope and inhibit signal transduction. Examples of binding fragments include, but are not limited to, a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (VH-CH1-VH-CH1) which, together with complementary light chain polypeptides, form a pair of antigen binding regions (Zapata et al., (1995) Protein Eng. 8:1057-1062; and U.S. Pat. No. 5,641,870).

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear,” “non-linear” or “conformational.” In one embodiment, the epitope is within domain 2 of HER3. In one embodiment, the epitope is a linear epitope within domain 2 of HER3. In one embodiment, the epitope is a non-linear epitope within domain 2 of HER3. In another embodiment, the epitope is a conformational epitope comprising amino acids residues within domain 2 of HER3. In one embodiment, the epitope comprises at least one of amino acid residue within domain 2 of HER3 (amino acids 208-328 of SEQ ID NO: 1), or a subset thereof. In one embodiment, the epitope comprises at least amino acid Lys268 (within domain 2) of SEQ ID NO:1. Antibodies or fragments thereof described herein can bind to Lys268 within domain 2 of HER3.

The term “linear epitope” refers to an epitope with all of the points of interaction between the protein and the interacting molecule (such as an antibody) occur linearly along the primary amino acid sequence of the protein (i.e., continuous amino acids). Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

The term “non-linear epitope” refers to epitope with non-contiguous amino acids that form a three-dimensional structure within a particular domain (e.g., within domain 1, within domain 2, within domain 3, or within domain 4). In one embodiment, the non-linear epitope is within domain 2. The non-linear epitope may also occur between two or more domains (e.g., the interface between domains 3-4). Non-linear epitope also refers to non-contiguous amino acids that are a result of a three-dimensional structure within a particular domain. The term “conformational epitope” refers to an epitope in which discontinuous amino acids that come together in three dimensional configuration involving at least two different domains, such as domain 2 and domain 4; or domain 3 and domain 4 In a conformational epitope, the points of interaction occur across amino acid residues on the protein that are separated from one another. As will be appreciated by one of skill in the art, the space that is occupied by a residue or side chain that creates the shape of a molecule helps to determine what an epitope is.

Generally, antibodies specific for a particular target antigen will preferentially recognize an epitope on the target antigen in a complex mixture of proteins and/or macromolecules.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., hydrogen/deuterium exchange, x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. MoI. Biol. 157:105-132; for hydropathy plots.

The phrases “monoclonal antibody” or “monoclonal antibody composition” as used herein refers to polypeptides, including antibodies, antibody fragments, bispecific antibodies, etc. that have substantially identical to amino acid sequence or are derived from the same genetic source. This term also includes preparations of antibody molecules of single molecular composition. A monoclonal antibody composition displays a single binding specificity and affinity for a particular epitope.

The phrase “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., (2000) J Mol Biol 296:57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit, NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing).

The phrase “human monoclonal antibody” as used herein refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The phrase “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Specific binding between two entities means a binding with an equilibrium constant (K_{A}) (kₒₙ/k_{off}) of at least 10²M⁻¹, at least 5×10²M⁻¹, at least 10³M⁻¹, at least 5×10³M⁻¹, at least 10⁴M⁻¹ at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹⁰M⁻¹, at least 10¹¹M⁻¹, at least 5×10¹¹M⁻¹, at least 10¹²M⁻¹, at least 5×10¹²M⁻¹, at least 10¹³M⁻¹, at least 5×10¹³ M⁻¹, at least 10¹⁴M⁻¹, at least 5×10¹⁴M⁻¹, at least 10¹⁵M⁻¹, or at least 5×10¹⁵M⁻¹.

The phrase “specifically (or selectively) binds” refers to a binding reaction of a HER3 binding antibody and HER3 receptor in a heterogeneous population of proteins and other biologics. In addition to the equilibrium constant (K_{A}) noted above, an HER3 binding antibody of the invention typically also has a dissociation rate constant (K_{D}) (k_{off}/kₒₙ) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower, and binds to HER3 with an affinity that is at least two-fold greater than its affinity for binding to a non-specific antigen (e.g., HSA).

In one embodiment, the antibody or fragment thereof has dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM, less than 10 pM, less than 1 pM as assessed using a method described herein or known to one of skill in the art (e.g., a BIAcore assay, ELISA, FACS, SET) (Biacore International AB, Uppsala, Sweden).

The term “K_{assoc}” or “Kₐ”, as used herein, refers to the association rate of a particular antibody-antigen interaction, whereas the term “K_{dis}” or “Kₐ,” as used herein, refers to the dissociation rate of a particular antibody-antigen interaction. The term “K_{D}”, as used herein, refers to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “avidity” as used herein refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

The term “valency” as used herein refers to the number of potential target binding sites in a polypeptide. Each target binding site specifically binds one target molecule or specific site (i.e., epitope) on a target molecule. When a polypeptide comprises more than one target binding site, each target binding site may specifically bind the same or different molecules (e.g., may bind to different molecules, e.g., different antigens, or different epitopes on the same molecule).

The phrase “inhibiting antibody” as used herein refers to an antibody that binds with HER3 and inhibits the biological activity of HER3 signaling, e.g., reduces, decreases and/or inhibits HER3 induced signaling activity, e.g., in a phospho-HER3 or phospho-Akt assay. Examples of assays are described in more details in the examples below. Accordingly, an antibody that “inhibits” one or more of these HER3 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). An antibody that inhibits HER3 activity effects such a statistically significant decrease by at least 10% of the measured parameter, by at least 50%, 80% or 90%, and in certain embodiments an antibody of the invention may inhibit greater than 95%, 98% or 99% of HER3 functional activity as evidenced by a reduction in the level of cellular HER3 phosphorylation.

The phrase “isolated antibody” refers to an antibody that is substantially free of other antibodies having different antigenic specificities (e.g., an isolated antibody that specifically binds HER3 is substantially free of antibodies that specifically bind antigens other than HER3). An isolated antibody that specifically binds HER3 may, however, have cross-reactivity to other antigens. Moreover, an isolated antibody may be substantially free of other cellular material and/or chemicals.

The phrase “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The terms “cross-compete” and “cross-competing” are used interchangeably herein to mean the ability of an antibody or fragment thereof to interfere with the binding of other antibodies or fragments thereof to HER3 in a standard competitive binding assay.

The ability or extent to which an antibody of fragment thereof is able to interfere with the binding of another antibody or fragment thereof to HER3, and therefore whether it can be said to cross-compete according to the invention, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-competing uses an ELISA-based approach.

The term “optimized” as used herein refers to a nucleotide sequence has been altered to encode an amino acid sequence using codons that are preferred in the production cell or organism, generally a eukaryotic cell, for example, a cell of Pichia, a cell of Trichoderma, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

Standard assays to evaluate the binding ability of the antibodies toward HER3 of various species are known in the art, including for example, ELISAs, western blots and RIAs. Suitable assays are described in detail in the Examples. The binding kinetics (e.g., binding affinity) of the antibodies also can be assessed by standard assays known in the art, such as by Biacore analysis, or FACS relative affinity (Scatchard). Assays to evaluate the effects of the antibodies on functional properties of HER3 (e.g., receptor binding assays, modulating the HER3 signal pathway) are described in further detail in the Examples.

The phrases “percent identical” or “percent identity,” in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The phrase “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “subject” as used herein includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “anti-cancer agent” as used herein refers to any agent that can be used to treat a cell proliferative disorder such as cancer, including cytotoxic agents, chemotherapeutic agents, radiotherapy and radiotherapeutic agents, targeted anti-cancer agents, and immunotherapeutic agents.

The term “tumor” as used herein refers to neoplastic cell growth and proliferation, whether malignant or benign, and all pre-cancerous and cancerous cells and tissues.

The term “anti-tumor activity” as used herein refers to a reduction in the rate of tumor cell proliferation, viability, or metastatic activity. A possible way of showing anti-tumor activity is show a decline in growth rate of abnormal cells that arises during therapy or tumor size stability or reduction. Such activity can be assessed using accepted in vitro or in vivo tumor models, including but not limited to xenograft models, allograft models, MMTV models, and other known models known in the art to investigate anti-tumor activity.

The term “malignancy” as used herein refers to a non-benign tumor or a cancer.

The term “cancer” as used herein refers to a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

Various aspects of the invention are described in further detail in the following sections and subsections.

### Structure and Mechanism of Activation of the HER Receptors

All four HER receptors have an extracellular ligand-binding domain, a single trans-membrane domain and a cytoplasmic tyrosine kinase-containing domain. The intracellular tyrosine kinase domain of HER receptors is highly conserved, although the kinase domain of HER3 contains substitutions of critical amino acids and therefore lacks kinase activity (Guy et al., (1994): PNAS 91, 8132-8136). Ligand-induced dimerisation of the HER receptors induces activation of the kinase, receptor transphosphorylation on tyrosine residues in the C-terminal tail, followed by recruitment and activation of intracellular signalling effectors (Yarden and Sliwkowski, (2001) Nature Rev 2, 127-137; Jorissen et al., (2003) Exp Cell Res 284, 31-53.

The crystal structures of the extracellular domains of HERs have provided some insight into the process of ligand-induced receptor activation (Schlessinger, (2002) Cell 110, 669-672). The extracellular domain of each HER receptor consists of four subdomains: Subdomain I and III cooperate in forming the ligand-binding site, whereas subdomain II (and perhaps also subdomain IV) participates in receptor dimerisation via direct receptor-receptor interactions. In the structures of ligand-bound HER1, a 13 hairpin (termed the dimerisation loop) in subdomain II interacts with the dimerisation loop of the partner receptor, mediating receptor dimerisation (Garrett et al, (2002) Cell 110, 763-773; Ogiso et al., (2002) Cell 110, 775-787). In contrast, in the structures of the inactive HER1, HER3 and HER4, the dimerisation loop is engaged in intramolecular interactions with subdomain IV, which prevents receptor dimerisation in the absence of ligand (Cho and Leahy, (2002) Science 297, 1330-1333; Ferguson et al., (2003) Mol Cell 12, 541-552; Bouyan et al., (2005) PNAS 102, 15024-15029). The structure of HER2 is unique among the HERs. In the absence of a ligand, HER2 has a conformation that resembles the ligand-activated state of HER1 with a protruding dimerisation loop, available to interact with other HER receptors (Cho et al., (2003) Nature 421, 756-760; Garrett et al., (2003) Mol Cell 11, 495-505). This may explain the enhanced heterodimerisation capacity of HER2.

Although the HER receptor crystal structures provide a model for HER receptor homo- and heterodimerisation, the background for the prevalence of some HER homo- and heterodimers over others (Franklin et al., (2004) Cancer Cell 5, 317-328) as well as the role of each of the domain in receptor dimerisation and autoinhibition (Burgess et al., (2003) Mol Cell 12, 541-552; Mattoon et al., (2004) PNAS 101, 923-928) remains somewhat unclear.

### HER3 Structure and Epitopes

A conformational epitope to which anti-HER3 antibodies bind has previously been described in PCT/EP2011/064407, and U.S. Ser. No. 61/375,408, both filed Aug. 22, 2011, and which are incorporated herein by reference in their entirety. The three dimensional structure of a truncated form of HER3 complexed with a HER3 antibody fragment, showed conformational epitope comprising domain 2 and domain 4 of HER3.

The present invention provides an additional class of antibodies or fragments thereof that bind a linear, non-linear, or conformational epitope within domain 2 of HER3. These antibodies or fragments thereof interact with HER3 to inhibit both ligand dependent and ligand independent signal transduction.

To examine the crystal structure of domain 2 antibodies or fragments thereof bound to HER3, crystals of HER3 may be prepared by expressing a nucleotide sequence encoding HER3 or a variant thereof in a suitable host cell, and then crystallising the purified protein(s) in the presence of the relevant HER3 targeted Fab. Preferably the HER3 polypeptide contains the extracellular domain (amino acids 20 to 640 of the human polypeptide (SEQ ID NO: 1) or a truncated version thereof, preferably comprising amino acids 20-640) but lacks the transmembrane and intracellular domains.

HER3 polypeptides may also be produced as fusion proteins, for example to aid in extraction and purification. Examples of fusion protein partners include glutathione-S-transferase (GST), histidine (HIS), hexahistidine (6HIS) (SEQ ID NO: 542), GAL4 (DNA binding and/or transcriptional activation domains) and beta-galactosidase. It may also be convenient to include a proteolytic cleavage site between the fusion protein partner and the protein sequence of interest to allow removal of fusion protein sequences.

After expression, the proteins may be purified and/or concentrated, for example by immobilised metal affinity chromatography, ion-exchange chromatography, and/or gel filtration.

The protein(s) may be crystallised using techniques described herein. Commonly, in a crystallisation process, a drop containing the protein solution is mixed with the crystallisation buffer and allowed to equilibrate in a sealed container. Equilibration may be achieved by known techniques such as the “hanging drop” or the “sitting drop” method. In these methods, the drop is hung above or sitting beside a much larger reservoir of crystallization buffer and equilibration is reached through vapor diffusion. Alternatively, equilibration may occur by other methods, for example under oil, through a semi-permeable membrane, or by free-interface diffusion (See e.g., Chayen et al., (2008) Nature Methods 5, 147-153.

Once the crystals have been obtained, the structure may be solved by known X-ray diffraction techniques. Many techniques use chemically modified crystals, such as those modified by heavy atom derivatization to approximate phases. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can then be determined by X-ray diffraction analysis of the soaked crystal. The patterns obtained on diffraction of a monochromatic beam of X-rays by the atoms (scattering centres) of the crystal can be solved by mathematical equations to give mathematical coordinates. The diffraction data are used to calculate an electron density map of the repeating unit of the crystal. Another method of obtaining phase information is using a technique known as molecular replacement. In this method, rotational and translational alogrithms are applied to a search model derived from a related structure, resulting in an approximate orientation for the protein of interest (See Rossmann, (1990) Acta Crystals A 46, 73-82). The electron density maps are used to establish the positions of the individual atoms within the unit cell of the crystal (Blundel et al., (1976) Protein Crystallography, Academic Press).

The approximate domain boundaries of extracellular domain of HER3 are as follows; domain 1: amino acids 20-207; domain 2: amino acids 208-328; domain 3: amino acids 329-498; and domain 4: amino acids 499-642. The three-dimensional structure of HER3 and the antibody allows the identification of target binding sites for potential HER3 modulators. Preferred target binding sites are those involved in the activation of HER3. In one embodiment, the target binding site is located within domain 2 of HER3. Thus an antibody or fragment thereof which binds to domain 2 can, for example, modulate HER3 activation by modifying the relative position of the domain relative to itself or other HER3 domains. Thus binding an antibody or fragment thereof to amino acid residues within domain 2 may cause the protein to adopt a configuration that prevents activation or prevent dimerisation with dimerizing partners (e.g., HER2).

In some embodiments, the antibody or fragment thereof recognize a specific conformational state of HER3 such that the antibody or fragment thereof prevents HER3 from interacting with a co-receptor (including, but not limited to, HER1, HER2 and HER4). In some embodiments, the antibody or fragment thereof prevents HER3 from interacting with a co-receptor by stabilizing the HER3 receptor in an inactive or closed state. In some embodiments, the antibody or fragment thereof may stabilize the HER3 receptor by binding to amino acid residues within domain 2 of HER3. In some embodiments, the antibody or fragment thereof binds to human HER3 protein having an epitope comprising HER3 amino acid residues within domain 2 (amino acids 208-328 of SEQ ID NO: 1), or a subset thereof. In some embodiments, the antibody or fragment thereof binds to amino acids within or overlapping amino acid residue within domain 2 (amino acids 208-328 of SEQ ID NO: 1). The antibody or fragment thereof described herein can bind to Lys 268 within domain 2 of HER3. In some embodiments, the antibody or fragment thereof binds to a linear epitope within domain 2 of HER3. In some embodiments, the antibody or fragment thereof binds to a non-linear epitope within domain 2 of HER3. In some embodiments, the antibody or fragment thereof binds to a conformational epitope within domain 2 of HER3.

In some embodiments, the antibody or fragment thereof binds to the epitope in domain 2 of HER3 such that the dimerization loop within domain 2 of HER3 is unavailable for dimerization with a co-receptor. The failure to form homo- or heterodimers results in failure to activate signal transduction.

In some embodiments, the antibody or fragment thereof can binds to an epitope in domain 2 in either the active or inactive state of HER3.

In some embodiments, the antibody or fragment thereof binds an epitope in domain 2 of HER3 receptor, where binding of the antibody or fragment thereof to the HER3 receptor allows dimerization with a co-receptor to form an inactive receptor-receptor complex. The formation of the inactive receptor-receptor complex prevents activation of ligand-independent signal transduction. For example, in ligand-independent signal transduction, HER3 may exists in an inactive state, however the overexpression of HER2 causes HER2-HER3 complex formation, however these resulting complexes are inactive and prevent activation of ligand-independent signal transduction.

In some embodiments, the domain(s)/region(s) containing residues that are in contact with or are buried by an antibody can be identified by mutating specific residues in HER3 (e.g., a wild-type antigen) and determining whether antibody or fragment thereof can bind the mutated or variant HER3 protein or measure changes of affinity from wild-type. By making a number of individual mutations, residues that play a direct role in binding or that are in sufficiently close proximity to the antibody such that a mutation can affect binding between the antibody and antigen can be identified. From a knowledge of these amino acids, the domain(s) or region(s) of the antigen (HER3) that contain residues in contact with the antibody or covered by the antibody can be elucidated. Mutagenesis using known techniques such as alanine-scanning can help define functionally relevant epitopes. Mutagenesis utilizing an arginine/glutamic acid scanning protocol can also be employed (see, e.g., Nanevicz et al., (1995), J. Biol. Chem. 270(37):21619-21625 and Zupnick et al., (2006), J. Biol. Chem. 281(29):20464-20473). In general, arginine and glutamic acids are substituted (typically individually) for an amino acid in the wild-type polypeptide because these amino acids are charged and bulky and thus have the potential to disrupt binding between an antigen binding protein and an antigen in the region of the antigen where the mutation is introduced. Arginines that exist in the wild-type antigen are replaced with glutamic acid. A variety of such individual mutants can be obtained and the collected binding results analyzed to determine what residues affect binding. A series of mutant HER3 antigens can be created, with each mutant antigen having a single mutation. Binding of each mutant HER3 antigen with various HER3 antibodies or fragments thereof can be measured and compared to the ability of the selected an antibody or fragments thereof to bind wild-type HER3 (SEQ ID NO: 1). Examples of such mutants are shown below in the Examples section, e.g., Lys 268 Als mutant.

An alteration (for example a reduction or increase) in binding between an antibody or fragment thereof and a mutant or variant HER3 as used herein means that there is a change in binding affinity (e.g., as measured by known methods such as Biacore testing or the bead based assay described below in the examples), EC₅₀, and/or a change (for example a reduction) in the total binding capacity of the antigen binding protein (for example, as evidenced by a decrease in Bₘₐₓ in a plot of antigen binding protein concentration versus antigen concentration). A significant alteration in binding indicates that the mutated residue is involved in binding to the antibody or fragment thereof.

In some embodiments, a significant reduction in binding means that the binding affinity, EC₅₀, and/or capacity between an antibody or fragments thereof and a mutant HER3 antigen is reduced by greater than 10%, greater than 20%, greater than 40%, greater than 50%, greater than 55%, greater than 60%, greater than 65%, greater than 70%, greater than 75%, greater than 80%, greater than 85%, greater than 90% or greater than 95% relative to binding between the an antibody or fragment thereof and a wild type HER3 (e.g., SEQ ID NO: 1).

In some embodiments, binding of an antibody or fragments thereof is significantly reduced or increased for a mutant HER3 protein having one or more (e.g., 1, 2, 3, 4, 5, 6, 7, 8, 9, 10, or more) mutations as compared to a wild-type HER3 protein (e.g., SEQ ID NO: 1).

Although the variant forms are referenced with respect to the wild-type sequence shown in SEQ ID NO: 1, it will be appreciated that in an allelic or splice variants of HER3 the amino acids could differ. Antibodies or fragments thereof showing significantly altered binding (e.g., lower or higher binding) for such allelic forms of HER3 are also contemplated.

In addition to the general structural aspects of antibodies, the more specific interaction between the paratope and the epitope may be examined through structural approaches. In one embodiment, the structure of the CDRs contribute to a paratope, through which an antibody is able to bind to an epitope. The shape of such a paratope may be determined in a number of ways. Traditional structural examination approaches can be used, such as NMR or x-ray crystallography. These approaches can examine the shape of the paratope alone, or while it is bound to the epitope. Alternatively, molecular models may be generated in silico. A structure can be generated through homology modeling, aided with a commercial package, such as InsightII modeling package from Accelrys (San Diego, Calif.). Briefly, one can use the sequence of the antibody to be examined to search against a database of proteins of known structures, such as the Protein Data Bank. After one identifies homologous proteins with known structures, these homologous proteins are used as modeling templates. Each of the possible templates can be aligned, thus producing structure based sequence alignments among the templates. The sequence of the antibody with the unknown structure can then be aligned with these templates to generate a molecular model for the antibody with the unknown structure. As will be appreciated by one of skill in the art, there are many alternative methods for generating such structures in silico, any of which may be used. For instance, a process similar to the one described in Hardman et al., issued U.S. Pat. No. 5,958,708 employing QUANTA (Polygen Corp., Waltham, Mass.) and CHARM (Brooks et al., (1983), J. Comp. Chem. 4:187) may be used (hereby incorporated in its entirety by reference).

Not only is the shape of the paratope important in determining whether and how well a possible paratope will bind to an epitope, but the interaction itself, between the epitope and the paratope is a source of great information in the design of variant antibodies. As appreciated by one of skill in the art, there are a variety of ways in which this interaction can be studied. One way is to use the structural model generated, perhaps as described above, and then to use a program such as InsightII (Accelrys, San Diego, Calif.), which has a docking module, which, among other things, is capable of performing a Monte Carlo search on the conformational and orientational spaces between the paratope and its epitope. The result is that one is able to estimate where and how the epitope interacts with the paratope. In one embodiment, only a fragment, or variant, of the epitope is used to assist in determining the relevant interactions. In one embodiment, the entire epitope is used in the modeling of the interaction between the paratope and the epitope.

Through the use of these modelled structures, one is able to predict which residues are the most important in the interaction between the epitope and the paratope. Thus, in one embodiment, one is able to readily select which residues to change in order to alter the binding characteristics of the antibody. For instance, it may be apparent from the docking models that the side chains of certain residues in the paratope may sterically hinder the binding of the epitope, thus altering these residues to residues with smaller side chains may be beneficial. One can determine this in many ways. For example, one may simply look at the two models and estimate interactions based on functional groups and proximity. Alternatively, one may perform repeated pairings of epitope and paratope, as described above, in order to obtain more favorable energy interactions. One can also determine these interactions for a variety of variants of the antibody to determine alternative ways in which the antibody may bind to the epitope. One can also combine the various models to determine how one should alter the structure of the antibodies in order to obtain an antibody with the particular characteristics that are desired.

The models determined above can be tested through various techniques. For example, the interaction energy can determined with the programs discussed above in order to determine which of the variants to further examine. Also, coulumbic and van der Waals interactions are used to determine the interaction energies of the epitope and the variant paratopes. Also site directed mutagenesis is used to see if predicted changes in antibody structure actually result in the desired changes in binding characteristics. Alternatively, changes may be made to the epitope to verify that the models are correct or to determine general binding themes that may be occurring between the paratope and the epitope.

As will be appreciated by one of skill in the art, while these models will provide the guidance necessary to make the antibodies and variants thereof of the present embodiments, it may still be desirable to perform routine testing of the in silico models, perhaps through in vitro studies. In addition, as will be apparent to one of skill in the art, any modification may also have additional side effects on the activity of the antibody. For instance, while any alteration predicted to result in greater binding, may induce greater binding, it may also cause other structural changes which might reduce or alter the activity of the antibody. The determination of whether or not this is the case is routine in the art and can be achieved in many ways. For example, the activity can be tested through an ELISA test. Alternatively, the samples can be tested through the use of a surface plasmon resonance device.

### HER3 Antibodies

The present invention provides antibodies that recognize an epitope within domain 2 of HER3. The invention is based on the surprising finding that a class of antibodies against HER3, block both ligand-dependent and ligand-independent HER3 signal transduction pathways. A class of antibodies that bind to an epitope within domain 2 of HER3 is disclosed in Table 1. In one embodiment, the antibodies inhibit both ligand-dependent and ligand-independent HER3 signalling. In another embodiment, the antibodies bind to HER3 and do not block HER ligand binding to the ligand binding site (i.e. both ligand and antibody can bind HER3 concurrently).

TABLE 1
Examples of HER3 Antibodies that bind to domain 2 of HER3

| | | |
|---|---|---|
| MOR12509 | | |
| SEQ ID NO: 2 (Kabat) | HCDR1 | NSWVA |
| SEQ ID NO: 3 (Kabat) | HCDR2 | IIYPGNSDTIYSPSFQG |
| SEQ ID NO: 4 (Kabat) | HCDR3 | VHIIQPPSAWSYNAMDV |
| SEQ ID NO: 5 (Chothia) | HCDR1 | GYSFTNS |
| SEQ ID NO: 6 (Chothia) | HCDR2 | YPGNSD |
| SEQ ID NO: 7 (Chothia) | HCDR3 | VHIIQPPSAWSYNAMDV |
| SEQ ID NO: 8 (Kabat) | LCDR1 | RASQSITHYLN |
| SEQ ID NO: 9 (Kabat) | LCDR2 | GASNLQS |
| SEQ ID NO: 10 (Kabat) | LCDR3 | QQAFVDPSTT |
| SEQ ID NO: 11 (Chothia) | LCDR1 | SQSITHY |
| SEQ ID NO: 12 (Chothia) | LCDR2 | GAS |
| SEQ ID NO: 13 (Chothia) | LCDR3 | AFVDPST |
| SEQ ID NO: 14 | VH | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSS |
| SEQ ID NO: 15 | VL | DIQMTQSPSSLSASVGDRVTITCRASQSITHYLNWYQQKPGKAPKLLIFGASNLQSGVPSRFSGSGS |
| | | GTDFTLTISSLQPEDFATYYCQQAFVDPSTTFGQGTKVEIK |
| SEQ ID NO: 16 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 17 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTACTCATTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGGCTTTCGTTGACCCGTCTACTACCTTTGGCCAGGGCACGAAAGTTGAAATT |
| | | AAA |
| SEQ ID NO: 18 | Heavy Chain | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 19 | Light Chain | DIQMTQSPSSLSASVGDRVTITCRASQSITHYLNWYQQKPGKAPKLLIFGASNLQSGVPSRFSGSGS |
| | | GTDFTLTISSLQPEDFATYYCQQAFVDPSTTFGQGTKVEIKRTVAAPSVFIFPPSDEQLKSGTASVVCL |
| | | LNNFYPREAKVQWKVDNALQSGNSQESVTEQDSKDSTYSLSSTLTLSKADYEKHKVYACEVTHQGL |
| | | SSPVTKSFNRGEC |
| SEQ ID NO: 20 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 21 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTACTCATTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGGCTTTCGTTGACCCGTCTACTACCTTTGGCCAGGGCACGAAAGTTGAAATT |
| | | AAACGTACGGTGGCCGCTCCCAGCGTGTTCATCTTCCCCCCCAGCGACGAGCAGCTGAAGAGCG |
| | | GCACCGCCAGCGTGGTGTGCCTGCTGAACAACTTCTACCCCCGGGAGGCCAAGGTGCAGTGGA |
| | | AGGTGGACAACGCCCTGCAGAGCGGCAACAGCCAGGAAAGCGTCACCGAGCAGGACAGCAAG |
| | | GACTCCACCTACAGCCTGAGCAGCACCCTGACCCTGAGCAAGGCCGACTACGAGAAGCACAAG |
| | | GTGTACGCCTGCGAGGTGACCCACCAGGGCCTGTCCAGCCCCGTGACCAAGAGCTTCAACCGG |
| | | GGCGAGTGT |
| MOR12510 | | |
| SEQ ID NO: 22 (Kabat) | HCDR1 | NSWVA |
| SEQ ID NO: 23 (Kabat) | HCDR2 | IIYPGNSDTIYSPSFQG |
| SEQ ID NO: 24 (Kabat) | HCDR3 | VHIIQPPSAWSYNAMDV |
| SEQ ID NO: 25 (Chothia) | HCDR1 | GYSFTNS |
| SEQ ID NO: 26 (Chothia) | HCDR2 | YPGNSD |
| SEQ ID NO: 27 (Chothia) | HCDR3 | VHIIQPPSAWSYNAMDV |
| SEQ ID NO: 28 (Kabat) | LCDR1 | RASQSISTYLN |
| SEQ ID NO: 29 (Kabat) | LCDR2 | GASNLQS |
| SEQ ID NO: 30 (Kabat) | LCDR3 | QQDIDEPWT |
| SEQ ID NO: 31 (Chothia) | LCDR1 | SQSISTY |
| SEQ ID NO: 32 (Chothia) | LCDR2 | GAS |
| SEQ ID NO: 33 (Chothia) | LCDR3 | DIDEPW |
| SEQ ID NO: 34 | VH | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSS |
| SEQ ID NO: 35 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQDIDEPWTFGQGTKVEIK |
| SEQ ID NO: 36 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 37 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGGACATCGACGAACCGTGGACCTTTGGCCAGGGCACGAAAGTTGAAATTA |
| | | AA |
| SEQ ID NO: 38 | Heavy Chain | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 39 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQDIDEPWTFGQGTKVEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSP |
| | | VTKSFNRGEC |
| SEQ ID NO: 40 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 41 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGGACATCGACGAACCGTGGACCTTTGGCCAGGGCACGAAAGTTGAAATTA |
| | | AACGTACGGTGGCCGCTCCCAGCGTGTTCATCTTCCCCCCCAGCGACGAGCAGCTGAAGAGCG |
| | | GCACCGCCAGCGTGGTGTGCCTGCTGAACAACTTCTACCCCCGGGAGGCCAAGGTGCAGTGGA |
| | | AGGTGGACAACGCCCTGCAGAGCGGCAACAGCCAGGAAAGCGTCACCGAGCAGGACAGCAAG |
| | | GACTCCACCTACAGCCTGAGCAGCACCCTGACCCTGAGCAAGGCCGACTACGAGAAGCACAAG |
| | | GTGTACGCCTGCGAGGTGACCCACCAGGGCCTGTCCAGCCCCGTGACCAAGAGCTTCAACCGG |
| | | GGCGAGTGT |
| MOR12616 | | |
| SEQ ID NO: 42 (Kabat) | HCDR1 | NSWVA |
| SEQ ID NO: 43 (Kabat) | HCDR2 | IIYPGNSDTIYSPSFQG |
| SEQ ID NO: 44 (Kabat) | HCDR3 | VHIIQPPSAWSYNAMDV |
| SEQ ID NO: 45 (Chothia) | HCDR1 | GYSFTNS |
| SEQ ID NO: 46 (Chothia) | HCDR2 | YPGNSD |
| SEQ ID NO: 47 (Chothia) | HCDR3 | VHIIQPPSAWSYNAMDV |
| SEQ ID NO: 48 (Kabat) | LCDR1 | RASQSISTYLN |
| SEQ ID NO: 49 (Kabat) | LCDR2 | GASNLQS |
| SEQ ID NO: 50 (Kabat) | LCDR3 | QQSITELFT |
| SEQ ID NO: 51 (Chothia) | LCDR1 | SQSISTY |
| SEQ ID NO: 52 (Chothia) | LCDR2 | GAS |
| SEQ ID NO: 53 (Chothia) | LCDR3 | SITELF |
| SEQ ID NO: 54 | VH | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSS |
| SEQ ID NO: 55 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 56 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 57 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 58 | Heavy Chain | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLOSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 59 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 60 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 61 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR12923 | | |
| SEQ ID NO: 62 (Kabat) | HCDR1 | NSWVA |
| SEQ ID NO: 63 (Kabat) | HCDR2 | IIYPGNSDTIYSPSFQG |
| SEQ ID NO: 64 (Kabat) | HCDR3 | VHIIQPPSAWSYNAMDV |
| SEQ ID NO: 65 (Chothia) | HCDR1 | GYSFTNS |
| SEQ ID NO: 66 (Chothia) | HCDR2 | YPGNSD |
| SEQ ID NO: 67 (Chothia) | HCDR3 | VHIIQPPSAWSYNAMDV |
| SEQ ID NO: 68 (Kabat) | LCDR1 | RASQSISTYLN |
| SEQ ID NO: 69 (Kabat) | LCDR2 | GASNLQS |
| SEQ ID NO: 70 (Kabat) | LCDR3 | QQSMYLPFT |
| SEQ ID NO: 71 (Chothia) | LCDR1 | SQSISTY |
| SEQ ID NO: 72 (Chothia) | LCDR2 | GAS |
| SEQ ID NO: 73 (Chothia) | LCDR3 | SMYLPF |
| SEQ ID NO: 74 | VH | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSS |
| SEQ ID NO: 75 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSMYLPFTFGQGTKVEIK |
| SEQ ID NO: 76 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 77 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATGTACCTGCCGTTCACCTTTGGCCAGGGCACGAAAGTTGAAATTAAA |
| SEQ ID NO: 78 | Heavy Chain | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 79 | Light Chain | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSMYLPFTFGQGTKVEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSP |
| | | VTKSFNRGEC |
| SEQ ID NO: 80 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 81 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATGTACCTGCCGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR12924 | | |
| SEQ ID NO: 82 (Kabat) | HCDR1 | NSWVA |
| SEQ ID NO: 83 (Kabat) | HCDR2 | IIYPGNSDTIYSPSFQG |
| SEQ ID NO: 84 (Kabat) | HCDR3 | VHIIQPPSAWSYNAMDV |
| SEQ ID NO: 85 (Chothia) | HCDR1 | GYSFTNS |
| SEQ ID NO: 86 (Chothia) | HCDR2 | YPGNSD |
| SEQ ID NO: 87 (Chothia) | HCDR3 | VHIIQPPSAWSYNAMDV |
| SEQ ID NO: 88 (Kabat) | LCDR1 | RASQSISTYLN |
| SEQ ID NO: 89 (Kabat) | LCDR2 | GASNLQS |
| SEQ ID NO: 90 (Kabat) | LCDR3 | QQSVWEPAT |
| SEQ ID NO: 91 (Chothia) | LCDR1 | SQSISTY |
| SEQ ID NO: 92 (Chothia) | LCDR2 | GAS |
| SEQ ID NO: 93 (Chothia) | LCDR3 | SVWEPA |
| SEQ ID NO: 94 | VH | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSS |
| SEQ ID NO: 95 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSVWEPATFGQGTKVEIK |
| SEQ ID NO: 96 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 97 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTGTTTGGGAACCGGCTACCTTTGGCCAGGGCACGAAAGTTGAAATTAA |
| | | A |
| SEQ ID NO: 98 | Heavy Chain | QVQLVQSGAEVKKPGESLKISCKGSGYSFTNSWVAWVRQMPGKGLEWMGIIYPGNSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 99 | Light Chain | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSVWEPATFGQGTKVEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSP |
| | | VTKSFNRGEC |
| SEQ ID NO: 100 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACTCTTGGGTTGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 101 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTGTTTGGGAACCGGCTACCTTTGGCCAGGGCACGAAAGTTGAAATTAA |
| | | ACGTACGGTGGCCGCTCCCAGCGTGTTCATCTTCCCCCCCAGCGACGAGCAGCTGAAGAGCGGC |
| | | ACCGCCAGCGTGGTGTGCCTGCTGAACAACTTCTACCCCCGGGAGGCCAAGGTGCAGTGGAAG |
| | | GTGGACAACGCCCTGCAGAGCGGCAACAGCCAGGAAAGCGTCACCGAGCAGGACAGCAAGGA |
| | | CTCCACCTACAGCCTGAGCAGCACCCTGACCCTGAGCAAGGCCGACTACGAGAAGCACAAGGT |
| | | GTACGCCTGCGAGGTGACCCACCAGGGCCTGTCCAGCCCCGTGACCAAGAGCTTCAACCGGGG |
| | | CGAGTGT |
| MOR12925 | | |
| SEQ ID NO: 102 (Kabat) | HCDR1 | NYWIA |
| SEQ ID NO: 103 (Kabat) | HCDR2 | IIYPGNSDTIYSPSFQG |
| SEQ ID NO: 104 (Kabat) | HCDR3 | VHIIQPPSAWSYNAMDV |
| SEQ ID NO: 105 (Chothia) | HCDR1 | GYSFSNY |
| SEQ ID NO: 106 (Chothia) | HCDR2 | YPGNSD |
| SEQ ID NO: 107 (Chothia) | HCDR3 | VHIIQPPSAWSYNAMDV |
| SEQ ID NO: 108 (Kabat) | LCDR1 | RASQSISTYLN |
| SEQ ID NO: 109 (Kabat) | LCDR2 | GASNLQS |
| SEQ ID NO: 110 (Kabat) | LCDR3 | QQSITELFT |
| SEQ ID NO: 111 (Chothia) | LCDR1 | SQSISTY |
| SEQ ID NO: 112 (Chothia) | LCDR2 | GAS |
| SEQ ID NO: 113 (Chothia) | LCDR3 | SITELF |
| SEQ ID NO: 114 | VH | QVQLVQSGAEVKKPGESLKISCKGSGYSFSNYWIAWVRQMPGKGLEWMGIIYPGNSDTIYSPSFQG |
| | | QVTISADKSISTAYLQWSSLKASDTAMYYCARVHIIQPPSAWSYNAMDVWGQGTLVTVSS |
| SEQ ID NO: 115 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 116 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 117 | DNA VL | GATATCGCGCTGACCCAGCCGGCGAGCGTGAGCGGTAGCCCGGGCCAGAGCATTACCATTAGC |
| | | TGCACCGGCACCAGCAGCGATGTGGGCACTTACAACCAGGTGTCTTGGTACCAGCAGCATCCG |
| | | GGCAAGGCGCCGAAACTGATGATCTACGGTGTTTCTAAACGTCCGAGCGGCGTGAGCAACCGT |
| | | TTTAGCGGATCCAAAAGCGGCAACACCGCGAGCCTGACCATTAGCGGCCTGCAAGCGGAAGAC |
| | | GAAGCGGATTATTACTGCCAGTCTCGTGGTGAATACCGTCCGGGTTGGGTGTTTGGCGGCGGC |
| | | ACGAAGTTAACCGTCCTA |
| SEQ ID NO: 118 | Heavy Chain | QVQLVQSGAEVKKPGESLKISCKGSGYSFSNYWIAWVRQMPGKGLEWMGIIYPGNSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 119 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 120 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTAACAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 121 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13750 | | |
| SEQ ID NO: 122 (Kabat) | HCDR1 | SSWVA |
| SEQ ID NO: 123 (Kabat) | HCDR2 | IIYPGGSDTIYSPSFQG |
| SEQ ID NO: 124 (Kabat) | HCDR3 | VHIIQPPSAWSYNAMDV |
| SEQ ID NO: 125 (Chothia) | HCDR1 | GYSFTSS |
| SEQ ID NO: 126 (Chothia) | HCDR2 | YPGGSD |
| SEQ ID NO: 127 (Chothia) | HCDR3 | VHIIQPPSAWSYNAMDV |
| SEQ ID NO: 128 (Kabat) | LCDR1 | RASQSISTYLN |
| SEQ ID NO: 129 (Kabat) | LCDR2 | GASNLQS |
| SEQ ID NO: 130 (Kabat) | LCDR3 | QQSITELFT |
| SEQ ID NO: 131 (Chothia) | LCDR1 | SQSISTY |
| SEQ ID NO: 132 (Chothia) | LCDR2 | GAS |
| SEQ ID NO: 133 (Chothia) | LCDR3 | SITELF |
| SEQ ID NO: 134 | VH | QVQLVQSGAEVKKPGESLKISCKGSGYSFTSSWVAWVRQMPGKGLEWMGIIYPGGSDTIYSPSFQ |
| | | GQVTISADKSISTAYLQWSSLKASDTAMYYCARVHIIQPPSAWSYNAMDVWGQGTLVTVSS |
| SEQ ID NO: 135 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 136 | DNA VH | CAGGTGCAATTGGTGCAGAGCGGTGCGGAAGTGAAAAAACCGGGTGCCAGCGTGAAAGTTAG |
| | | CTGCAAAGCGTCCGGATATACCTTCACTTCTTACGACATCCATTGGGTGCGCCAGGCCCCGGGC |
| | | CAGGGCCTCGAGTGGATGGGCCGTATCGACCCGTACTCTGGCAACACGAACTACGCGCAGAAA |
| | | TTTCAGGGCCGGGTGACCATGACCCGTGATACCAGCATTAGCACCGCGTATATGGAACTGAGCC |
| | | GTCTGCGTAGCGAAGATACGGCCGTGTATTATTGCGCGCGTGGTTCTTTCTACACTCGTGACTCT |
| | | TACTTCGATGTTTGGGGCCAAGGCACCCTGGTGACTGTTAGCTCA |
| SEQ ID NO: 137 | DNA VL | GATATCGCGCTGACCCAGCCGGCGAGCGTGAGCGGTAGCCCGGGCCAGAGCATTACCATTAGC |
| | | TGCACCGGCACCAGCAGCGATGTGGGCACTTACAACCAGGTGTCTTGGTACCAGCAGCATCCG |
| | | GGCAAGGCGCCGAAACTGATGATCTACGGTGTTTCTAAACGTCCGAGCGGCGTGAGCAACCGT |
| | | TTTAGCGGATCCAAAAGCGGCAACACCGCGAGCCTGACCATTAGCGGCCTGCAAGCGGAAGAC |
| | | GAAGCGGATTATTACTGCCAGTCTCGTGGTGAATACCGTCCGGGTTGGGTGTTTGGCGGCGGC |
| | | ACGAAGTTAACCGTCCTA |
| SEQ ID NO: 138 | Heavy Chain | QVQLVQSGAEVKKPGESLKISCKGSGYSFTSSWVAWVRQMPGKGLEWMGIIYPGGSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 139 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 140 | DNA Heavy | CAGGTGCAATTGGTGCAGAGCGGTGCGGAAGTGAAAAAACCGGGTGCCAGCGTGAAAGTTAG |
| | Chain | CTGCAAAGCGTCCGGATATACCTTCACTTCTTACGACATCCATTGGGTGCGCCAGGCCCCGGGC |
| | | CAGGGCCTCGAGTGGATGGGCCGTATCGACCCGTACTCTGGCAACACGAACTACGCGCAGAAA |
| | | TTTCAGGGCCGGGTGACCATGACCCGTGATACCAGCATTAGCACCGCGTATATGGAACTGAGCC |
| | | GTCTGCGTAGCGAAGATACGGCCGTGTATTATTGCGCGCGTGGTTCTTTCTACACTCGTGACTCT |
| | | TACTTCGATGTTTGGGGCCAAGGCACCCTGGTGACTGTTAGCTCAGCCTCCACCAAGGGTCCAT |
| | | CGGTCTTCCCCCTGGCACCCTCCTCCAAGAGCACCTCTGGGGGCACAGCGGCCCTGGGCTGCCT |
| | | GGTCAAGGACTACTTCCCCGAACCGGTGACGGTGTCGTGGAACTCAGGCGCCCTGACCAGCGG |
| | | CGTGCACACCTTCCCGGCTGTCCTACAGTCCTCAGGACTCTACTCCCTCAGCAGCGTGGTGACCG |
| | | TGCCCTCCAGCAGCTTGGGCACCCAGACCTACATCTGCAACGTGAATCACAAGCCCAGCAACAC |
| | | CAAGGTGGACAAGAGAGTTGAGCCCAAATCTTGTGACAAAACTCACACATGCCCACCGTGCCCA |
| | | GCACCTGAACTCCTGGGGGGACCGTCAGTCTTCCTCTTCCCCCCAAAACCCAAGGACACCCTCAT |
| | | GATCTCCCGGACCCCTGAGGTCACATGCGTGGTGGTGGACGTGAGCCACGAAGACCCTGAGGT |
| | | CAAGTTCAACTGGTACGTGGACGGCGTGGAGGTGCATAATGCCAAGACAAAGCCGCGGGAGG |
| | | AGCAGTACAACAGCACGTACCGGGTGGTCAGCGTCCTCACCGTCCTGCACCAGGACTGGCTGA |
| | | ATGGCAAGGAGTACAAGTGCAAGGTCTCCAACAAAGCCCTCCCAGCCCCCATCGAGAAAACCAT |
| | | CTCCAAAGCCAAAGGGCAGCCCCGAGAACCACAGGTGTACACCCTGCCCCCATCCCGGGAGGA |
| | | GATGACCAAGAACCAGGTCAGCCTGACCTGCCTGGTCAAAGGCTTCTATCCCAGCGACATCGCC |
| | | GTGGAGTGGGAGAGCAATGGGCAGCCGGAGAACAACTACAAGACCACGCCTCCCGTGCTGGA |
| | | CTCCGACGGCTCCTTCTTCCTCTACAGCAAGCTCACCGTGGACAAGAGCAGGTGGCAGCAGGG |
| | | GAACGTCTTCTCATGCTCCGTGATGCATGAGGCTCTGCACAACCACTACACGCAGAAGAGCCTC |
| | | TCCCTGTCTCCGGGTAAA |
| SEQ ID NO: 141 | DNA Light | GATATCGCGCTGACCCAGCCGGCGAGCGTGAGCGGTAGCCCGGGCCAGAGCATTACCATTAGC |
| | Chain | TGCACCGGCACCAGCAGCGATGTGGGCACTTACAACCAGGTGTCTTGGTACCAGCAGCATCCG |
| | | GGCAAGGCGCCGAAACTGATGATCTACGGTGTTTCTAAACGTCCGAGCGGCGTGAGCAACCGT |
| | | TTTAGCGGATCCAAAAGCGGCAACACCGCGAGCCTGACCATTAGCGGCCTGCAAGCGGAAGAC |
| | | GAAGCGGATTATTACTGCCAGTCTCGTGGTGAATACCGTCCGGGTTGGGTGTTTGGCGGCGGC |
| | | ACGAAGTTAACCGTCCTAGGTCAGCCCAAGGCTGCCCCCTCGGTCACTCTGTTCCCGCCCTCCTC |
| | | TGAGGAGCTTCAAGCCAACAAGGCCACACTGGTGTGTCTCATAAGTGACTTCTACCCGGGAGCC |
| | | GTGACAGTGGCCTGGAAGGCAGATAGCAGCCCCGTCAAGGCGGGAGTGGAGACCACCACACC |
| | | CTCCAAACAAAGCAACAACAAGTACGCGGCCAGCAGCTATCTGAGCCTGACGCCTGAGCAGTG |
| | | GAAGTCCCACAGAAGCTACAGCTGCCAGGTCACGCATGAAGGGAGCACCGTGGAGAAGACAG |
| | | TGGCCCCTACAGAATGTTCA |
| MOR13752 | | |
| SEQ ID NO: 142 (Kabat) | HCDR1 | NYWVA |
| SEQ ID NO: 143 (Kabat) | HCDR2 | IIYPGGSDTIYSPSFQG |
| SEQ ID NO: 144 (Kabat) | HCDR3 | VHIIQPPSAWSYNAMDV |
| SEQ ID NO: 145 (Chothia) | HCDR1 | GYSFTNY |
| SEQ ID NO: 146 (Chothia) | HCDR2 | YPGGSD |
| SEQ ID NO: 147 (Chothia) | HCDR3 | VHIIQPPSAWSYNAMDV |
| SEQ ID NO: 148 (Kabat) | LCDR1 | RASQSISTYLN |
| SEQ ID NO: 149 (Kabat) | LCDR2 | GASNLQS |
| SEQ ID NO: 150 (Kabat) | LCDR3 | QQSITELFT |
| SEQ ID NO: 151 (Chothia) | LCDR1 | SQSISTY |
| SEQ ID NO: 152 (Chothia) | LCDR2 | GAS |
| SEQ ID NO: 153 (Chothia) | LCDR3 | SITELF |
| SEQ ID NO: 154 | VH | QVQLVQSGAEVKKPGESLKISCKGSGYSFTNYWVAWVRQMPGKGLEWMGIIYPGGSDTIYSPSFQ |
| | | GQVTISADKSISTAYLQWSSLKASDTAMYYCARVHIIQPPSAWSYNAMDVWGQGTLVTVSS |
| SEQ ID NO: 155 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 156 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACTATTGGGTTGCTTGGGTGCGCCAGATGCCGGGC |
| | | AAAGGTCTCGAGTGGATGGGCATCATCTACCCGGGTGGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCA |
| SEQ ID NO: 157 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 158 | Heavy Chain | QVQLVQSGAEVKKPGESLKISCKGSGYSFTNYWVAWVRQMPGKGLEWMGIIYPGGSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 159 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 160 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACTATTGGGTTGCTTGGGTGCGCCAGATGCCGGGC |
| | | AAAGGTCTCGAGTGGATGGGCATCATCTACCCGGGTGGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCAGC |
| | | CTCCACCAAGGGTCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTGAAT |
| | | CACAAGCCCAGCAACACCAAGGTGGACAAGAGAGTTGAGCCCAAATCTTGTGACAAAACTCAC |
| | | ACATGCCCACCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCAA |
| | | GACAAAGCCGCGGGAGGAGCAGTACAACAGCACGTACCGGGTGGTCAGCGTCCTCACCGTCCT |
| | | GCACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCAAAGGCTT |
| | | CTATCCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| SEQ ID NO: 161 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13754 | | |
| SEQ ID NO: 162 (Kabat) | HCDR1 | SYWVA |
| SEQ ID NO: 163 (Kabat) | HCDR2 | IIYPGGSDTIYSPSFQG |
| SEQ ID NO: 164 (Kabat) | HCDR3 | VHIIQPPSAWSYNAMDV |
| SEQ ID NO: 165 (Chothia) | HCDR1 | GYSFTSY |
| SEQ ID NO: 166 (Chothia) | HCDR2 | YPGGSD |
| SEQ ID NO: 167 (Chothia) | HCDR3 | VHIIQPPSAWSYNAMDV |
| SEQ ID NO: 168 (Kabat) | LCDR1 | RASQSISTYLN |
| SEQ ID NO: 169 (Kabat) | LCDR2 | GASNLQS |
| SEQ ID NO: 170 (Kabat) | LCDR3 | QQSITELFT |
| SEQ ID NO: 171 (Chothia) | LCDR1 | SQSISTY |
| SEQ ID NO: 172 (Chothia) | LCDR2 | GAS |
| SEQ ID NO: 173 (Chothia) | LCDR3 | SITELF |
| SEQ ID NO: 174 | VH | QVQLVQSGAEVKKPGESLKISCKGSGYSFTSYWVAWVRQMPGKGLEWMGIIYPGGSDTIYSPSFQ |
| | | GQVTISADKSISTAYLQWSSLKASDTAMYYCARVHIIQPPSAWSYNAMDVWGQGTLVTVSS |
| SEQ ID NO: 175 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 176 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAGCTATTGGGTTGCTTGGGTGCGCCAGATGCCGGGC |
| | | AAAGGTCTCGAGTGGATGGGCATCATCTACCCGGGTGGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCA |
| SEQ ID NO: 177 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 178 | Heavy Chain | QVQLVQSGAEVKKPGESLKISCKGSGYSFTSYWVAWVRQMPGKGLEWMGIIYPGGSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 179 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 180 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAGCTATTGGGTTGCTTGGGTGCGCCAGATGCCGGGC |
| | | AAAGGTCTCGAGTGGATGGGCATCATCTACCCGGGTGGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCAGC |
| | | CTCCACCAAGGGTCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTGAAT |
| | | CACAAGCCCAGCAACACCAAGGTGGACAAGAGAGTTGAGCCCAAATCTTGTGACAAAACTCAC |
| | | ACATGCCCACCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCAA |
| | | GACAAAGCCGCGGGAGGAGCAGTACAACAGCACGTACCGGGTGGTCAGCGTCCTCACCGTCCT |
| | | GCACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCAAAGGCTT |
| | | CTATCCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| SEQ ID NO: 181 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13755 | | |
| SEQ ID NO: 182 (Kabat) | HCDR1 | TYWVA |
| SEQ ID NO: 183 (Kabat) | HCDR2 | IIYPGQSDTIYSPSFQG |
| SEQ ID NO: 184 (Kabat) | HCDR3 | VHIIQPPSAWSYNAMDV |
| SEQ ID NO: 185 (Chothia) | HCDR1 | GYSFTTY |
| SEQ ID NO: 186 (Chothia) | HCDR2 | YPGQSD |
| SEQ ID NO: 187 (Chothia) | HCDR3 | VHIIQPPSAWSYNAMDV |
| SEQ ID NO: 188 (Kabat) | LCDR1 | RASQSISTYLN |
| SEQ ID NO: 189 (Kabat) | LCDR2 | GASNLQS |
| SEQ ID NO: 190 (Kabat) | LCDR3 | QQSITELFT |
| SEQ ID NO: 191 (Chothia) | LCDR1 | SQSISTY |
| SEQ ID NO: 192 (Chothia) | LCDR2 | GAS |
| SEQ ID NO: 193 (Chothia) | LCDR3 | SITELF |
| SEQ ID NO: 194 | VH | QVQLVQSGAEVKKPGESLKISCKGSGYSFTTYWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 195 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 196 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTACCTATTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 197 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 198 | Heavy Chain | QVQLVQSGAEVKKPGESLKISCKGSGYSFTTYWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLOSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 199 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 200 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTACCTATTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 201 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13756 | | |
| SEQ ID NO: 202 (Kabat) | HCDR1 | LTWVA |
| SEQ ID NO: 203 (Kabat) | HCDR2 | IIYPGQSDTIYSPSFQG |
| SEQ ID NO: 204 (Kabat) | HCDR3 | VHIIQPPSAWSYNAMDV |
| SEQ ID NO: 205 (Chothia) | HCDR1 | GYSFTLT |
| SEQ ID NO: 206 (Chothia) | HCDR2 | YPGQSD |
| SEQ ID NO: 207 (Chothia) | HCDR3 | VHIIQPPSAWSYNAMDV |
| SEQ ID NO: 208 (Kabat) | LCDR1 | RASQSISTYLN |
| SEQ ID NO: 209 (Kabat) | LCDR2 | GASNLQS |
| SEQ ID NO: 210 (Kabat) | LCDR3 | QQSITELFT |
| SEQ ID NO: 211 (Chothia) | LCDR1 | SQSISTY |
| SEQ ID NO: 212 (Chothia) | LCDR2 | GAS |
| SEQ ID NO: 213 (Chothia) | LCDR3 | SITELF |
| SEQ ID NO: 214 | VH | QVQLVQSGAEVKKPGESLKISCKGSGYSFTLTWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 215 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 216 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTCTCACCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 217 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 218 | Heavy Chain | QVQLVQSGAEVKKPGESLKISCKGSGYSFTLTWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 219 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 220 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTCTCACCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 221 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13758 | | |
| SEQ ID NO: 222 (Kabat) | HCDR1 | LYWVA |
| SEQ ID NO: 223 (Kabat) | HCDR2 | IIYPGQSDTIYSPSFQG |
| SEQ ID NO: 224 (Kabat) | HCDR3 | VHIIQPPSAWSYNAMDV |
| SEQ ID NO: 225 (Chothia) | HCDR1 | GYSFTLY |
| SEQ ID NO: 226 (Chothia) | HCDR2 | YPGQSD |
| SEQ ID NO: 227 (Chothia) | HCDR3 | VHIIQPPSAWSYNAMDV |
| SEQ ID NO: 228 (Kabat) | LCDR1 | RASQSISTYLN |
| SEQ ID NO: 229 (Kabat) | LCDR2 | GASNLQS |
| SEQ ID NO: 230 (Kabat) | LCDR3 | QQSITELFT |
| SEQ ID NO: 231 (Chothia) | LCDR1 | SQSISTY |
| SEQ ID NO: 232 (Chothia) | LCDR2 | GAS |
| SEQ ID NO: 233 (Chothia) | LCDR3 | SITELF |
| SEQ ID NO: 234 | VH | QVQLVQSGAEVKKPGESLKISCKGSGYSFTLYWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 235 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 236 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTCTCTACTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 237 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 238 | Heavy Chain | QVQLVQSGAEVKKPGESLKISCKGSGYSFTLYWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 239 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 240 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTCTCTACTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 241 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13759 | | |
| SEQ ID NO: 242 (Kabat) | HCDR1 | TTWVA |
| SEQ ID NO: 243 (Kabat) | HCDR2 | IIYPGLSDTIYSPSFQG |
| SEQ ID NO: 244 (Kabat) | HCDR3 | VHIIQPPSAWSYNAMDV |
| SEQ ID NO: 245 (Chothia) | HCDR1 | GYSFTTT |
| SEQ ID NO: 246 (Chothia) | HCDR2 | YPGLSD |
| SEQ ID NO: 247 (Chothia) | HCDR3 | VHIIQPPSAWSYNAMDV |
| SEQ ID NO: 248 (Kabat) | LCDR1 | RASQSISTYLN |
| SEQ ID NO: 249 (Kabat) | LCDR2 | GASNLQS |
| SEQ ID NO: 250 (Kabat) | LCDR3 | QQSITELFT |
| SEQ ID NO: 251 (Chothia) | LCDR1 | SQSISTY |
| SEQ ID NO: 252 (Chothia) | LCDR2 | GAS |
| SEQ ID NO: 253 (Chothia) | LCDR3 | SITELF |
| SEQ ID NO: 254 | VH | QVQLVQSGAEVKKPGESLKISCKGSGYSFTTTWVAWVRQMPGKGLEWMGIIYPGLSDTIYSPSFQG |
| | | QVTISADKSISTAYLQWSSLKASDTAMYYCARVHIIQPPSAWSYNAMDVWGQGTLVTVSS |
| SEQ ID NO: 255 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 256 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTACCACTTGGGTTGCTTGGGTGCGCCAGATGCCGGGC |
| | | AAAGGTCTCGAGTGGATGGGCATCATCTACCCGGGTCTGAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 257 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 258 | Heavy Chain | QVQLVQSGAEVKKPGESLKISCKGSGYSFTTTWVAWVRQMPGKGLEWMGIIYPGLSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 259 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 260 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTACCACTTGGGTTGCTTGGGTGCGCCAGATGCCGGGC |
| | | AAAGGTCTCGAGTGGATGGGCATCATCTACCCGGGTCTGAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 261 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13761 | | |
| SEQ ID NO: 262 (Kabat) | HCDR1 | QTWVA |
| SEQ ID NO: 263 (Kabat) | HCDR2 | IIYPGQSDTIYSPSFQG |
| SEQ ID NO: 264 (Kabat) | HCDR3 | VHIIQPPSAWSYNAMDV |
| SEQ ID NO: 265 (Chothia) | HCDR1 | GYSFTQT |
| SEQ ID NO: 266 (Chothia) | HCDR2 | YPGQSD |
| SEQ ID NO: 267 (Chothia) | HCDR3 | VHIIQPPSAWSYNAMDV |
| SEQ ID NO: 268 (Kabat) | LCDR1 | RASQSISTYLN |
| SEQ ID NO: 269 (Kabat) | LCDR2 | GASNLQS |
| SEQ ID NO: 270 (Kabat) | LCDR3 | QQSITELFT |
| SEQ ID NO: 271 (Chothia) | LCDR1 | SQSISTY |
| SEQ ID NO: 272 (Chothia) | LCDR2 | GAS |
| SEQ ID NO: 273 (Chothia) | LCDR3 | SITELF |
| SEQ ID NO: 274 | VH | QVQLVQSGAEVKKPGESLKISCKGSGYSFTQTWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 275 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 276 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTCAGACCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 277 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 278 | Heavy Chain | QVQLVQSGAEVKKPGESLKISCKGSGYSFTQTWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 279 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 280 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTCAGACCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 281 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13762 | | |
| SEQ ID NO: 282 (Kabat) | HCDR1 | NTWVA |
| SEQ ID NO: 283 (Kabat) | HCDR2 | IIYPGQSDTIYSPSFQG |
| SEQ ID NO: 284 (Kabat) | HCDR3 | VHIIQPPSAWSYNAMDV |
| SEQ ID NO: 285 (Chothia) | HCDR1 | GYSFTNT |
| SEQ ID NO: 286 (Chothia) | HCDR2 | YPGQSD |
| SEQ ID NO: 287 (Chothia) | HCDR3 | VHIIQPPSAWSYNAMDV |
| SEQ ID NO: 288 (Kabat) | LCDR1 | RASQSISTYLN |
| SEQ ID NO: 289 (Kabat) | LCDR2 | GASNLQS |
| SEQ ID NO: 290 (Kabat) | LCDR3 | QQSITELFT |
| SEQ ID NO: 291 (Chothia) | LCDR1 | SQSISTY |
| SEQ ID NO: 292 (Chothia) | LCDR2 | GAS |
| SEQ ID NO: 293 (Chothia) | LCDR3 | SITELF |
| SEQ ID NO: 294 | VH | QVQLVQSGAEVKKPGESLKISCKGSGYSFTNTWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 295 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 296 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAACACCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 297 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 298 | Heavy Chain | QVQLVQSGAEVKKPGESLKISCKGSGYSFTNTWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLOSSGLYSLSSVVIVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 299 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 300 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAACACCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 301 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13763 | | |
| SEQ ID NO: 302 (Kabat) | HCDR1 | SYWVA |
| SEQ ID NO: 303 (Kabat) | HCDR2 | IIYPGQSDTIYSPSFQG |
| SEQ ID NO: 304 (Kabat) | HCDR3 | VHIIQPPSAWSYNAMDV |
| SEQ ID NO: 305 (Chothia) | HCDR1 | GYSFTSY |
| SEQ ID NO: 306 (Chothia) | HCDR2 | YPGQSD |
| SEQ ID NO: 307 (Chothia) | HCDR3 | VHIIQPPSAWSYNAMDV |
| SEQ ID NO: 308 (Kabat) | LCDR1 | RASQSISTYLN |
| SEQ ID NO: 309 (Kabat) | LCDR2 | GASNLQS |
| SEQ ID NO: 310 (Kabat) | LCDR3 | QQSITELFT |
| SEQ ID NO: 311 (Chothia) | LCDR1 | SQSISTY |
| SEQ ID NO: 312 (Chothia) | LCDR2 | GAS |
| SEQ ID NO: 313 (Chothia) | LCDR3 | SITELF |
| SEQ ID NO: 314 | VH | QVQLVQSGAEVKKPGESLKISCKGSGYSFTSYWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 315 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 316 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAGCTACTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 317 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 318 | Heavy Chain | QVQLVQSGAEVKKPGESLKISCKGSGYSFTSYWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 319 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 320 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAGCTACTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 321 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13765 | | |
| SEQ ID NO: 322 (Kabat) | HCDR1 | GSWVA |
| SEQ ID NO: 323 (Kabat) | HCDR2 | IIYPGTSDTIYSPSFQG |
| SEQ ID NO: 324 (Kabat) | HCDR3 | VHIIQPPSAWSYNAMDV |
| SEQ ID NO: 325 (Chothia) | HCDR1 | GYSFTGS |
| SEQ ID NO: 326 (Chothia) | HCDR2 | YPGTSD |
| SEQ ID NO: 327 (Chothia) | HCDR3 | VHIIQPPSAWSYNAMDV |
| SEQ ID NO: 328 (Kabat) | LCDR1 | RASQSISTYLN |
| SEQ ID NO: 329 (Kabat) | LCDR2 | GASNLQS |
| SEQ ID NO: 330 (Kabat) | LCDR3 | QQSITELFT |
| SEQ ID NO: 331 (Chothia) | LCDR1 | SQSISTY |
| SEQ ID NO: 332 (Chothia) | LCDR2 | GAS |
| SEQ ID NO: 333 (Chothia) | LCDR3 | SITELF |
| SEQ ID NO: 334 | VH | QVQLVQSGAEVKKPGESLKISCKGSGYSFTGSWVAWVRQMPGKGLEWMGIIYPGTSDTIYSPSFQ |
| | | GQVTISADKSISTAYLQWSSLKASDTAMYYCARVHIIQPPSAWSYNAMDVWGQGTLVTVSS |
| SEQ ID NO: 335 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 336 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTGGCAGCTGGGTTGCTTGGGTGCGCCAGATGCCGGG |
| | | CAAAGGTCTCGAGTGGATGGGCATCATCTACCCGGGTACCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCA |
| SEQ ID NO: 337 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 338 | Heavy Chain | QVQLVQSGAEVKKPGESLKISCKGSGYSFTGSWVAWVRQMPGKGLEWMGIIYPGTSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 339 | Light Chain | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 340 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTGGCAGCTGGGTTGCTTGGGTGCGCCAGATGCCGGG |
| | | CAAAGGTCTCGAGTGGATGGGCATCATCTACCCGGGTACCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCAGC |
| | | CTCCACCAAGGGTCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTGAAT |
| | | CACAAGCCCAGCAACACCAAGGTGGACAAGAGAGTTGAGCCCAAATCTTGTGACAAAACTCAC |
| | | ACATGCCCACCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCAA |
| | | GACAAAGCCGCGGGAGGAGCAGTACAACAGCACGTACCGGGTGGTCAGCGTCCTCACCGTCCT |
| | | GCACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCAAAGGCTT |
| | | CTATCCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| SEQ ID NO: 341 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13766 | | |
| SEQ ID NO: 342 (Kabat) | HCDR1 | TTWVA |
| SEQ ID NO: 343 (Kabat) | HCDR2 | IIYPGSSDTIYSPSFQG |
| SEQ ID NO: 344 (Kabat) | HCDR3 | VHIIQPPSAWSYNAMDV |
| SEQ ID NO: 345 (Chothia) | HCDR1 | GYSFTTT |
| SEQ ID NO: 346 (Chothia) | HCDR2 | YPGSSD |
| SEQ ID NO: 347 (Chothia) | HCDR3 | VHIIQPPSAWSYNAMDV |
| SEQ ID NO: 348 (Kabat) | LCDR1 | RASQSISTYLN |
| SEQ ID NO: 349 (Kabat) | LCDR2 | GASNLQS |
| SEQ ID NO: 350 (Kabat) | LCDR3 | QQSITELFT |
| SEQ ID NO: 351 (Chothia) | LCDR1 | SQSISTY |
| SEQ ID NO: 352 (Chothia) | LCDR2 | GAS |
| SEQ ID NO: 353 (Chothia) | LCDR3 | SITELF |
| SEQ ID NO: 354 | VH | QVQLVQSGAEVKKPGESLKISCKGSGYSFTTTWVAWVRQMPGKGLEWMGIIYPGSSDTIYSPSFQG |
| | | QVTISADKSISTAYLQWSSLKASDTAMYYCARVHIIQPPSAWSYNAMDVWGQGTLVTVSS |
| SEQ ID NO: 355 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 356 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTACCACCTGGGTTGCTTGGGTGCGCCAGATGCCGGGC |
| | | AAAGGTCTCGAGTGGATGGGCATCATCTACCCGGGTAGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCA |
| SEQ ID NO: 357 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 358 | Heavy Chain | QVQLVQSGAEVKKPGESLKISCKGSGYSFTTTWVAWVRQMPGKGLEWMGIIYPGSSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| SEQ ID NO: 359 | Light Chain | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| | | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 360 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTACCACCTGGGTTGCTTGGGTGCGCCAGATGCCGGGC |
| | | AAAGGTCTCGAGTGGATGGGCATCATCTACCCGGGTAGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCAGC |
| | | CTCCACCAAGGGTCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTGAAT |
| | | CACAAGCCCAGCAACACCAAGGTGGACAAGAGAGTTGAGCCCAAATCTTGTGACAAAACTCAC |
| | | ACATGCCCACCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCAA |
| | | GACAAAGCCGCGGGAGGAGCAGTACAACAGCACGTACCGGGTGGTCAGCGTCCTCACCGTCCT |
| | | GCACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCAAAGGCTT |
| | | CTATCCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| SEQ ID NO: 361 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13767 | | |
| SEQ ID NO: 362 (Kabat) | HCDR1 | TSWVA |
| SEQ ID NO: 363 (Kabat) | HCDR2 | IIYPGQSDTIYSPSFQG |
| SEQ ID NO: 364 (Kabat) | HCDR3 | VHIIQPPSAWSYNAMDV |
| SEQ ID NO: 365 (Chothia) | HCDR1 | GYSFTTS |
| SEQ ID NO: 366 (Chothia) | HCDR2 | YPGQSD |
| SEQ ID NO: 367 (Chothia) | HCDR3 | VHIIQPPSAWSYNAMDV |
| SEQ ID NO: 368 (Kabat) | LCDR1 | RASQSISTYLN |
| SEQ ID NO: 369 (Kabat) | LCDR2 | GASNLQS |
| SEQ ID NO: 370 (Kabat) | LCDR3 | QQSITELFT |
| SEQ ID NO: 371 (Chothia) | LCDR1 | SQSISTY |
| SEQ ID NO: 372 (Chothia) | LCDR2 | GAS |
| SEQ ID NO: 373 (Chothia) | LCDR3 | SITELF |
| SEQ ID NO: 374 | VH | QVQLVQSGAEVKKPGESLKISCKGSGYSFTTSWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 375 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 376 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTACCAGCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 377 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| SEQ ID NO: 378 | Heavy Chain | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | QVQLVQSGAEVKKPGESLKISCKGSGYSFTTSWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLQSSGLYSLSSVVTVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 379 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 380 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTACCAGCTGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 381 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13768 | | |
| SEQ ID NO: 382 (Kabat) | HCDR1 | SSWVA |
| SEQ ID NO: 383 (Kabat) | HCDR2 | IlYPGQSDTIYSPSFQG |
| SEQ ID NO: 384 (Kabat) | HCDR3 | VHIIQPPSAWSYNAMDV |
| SEQ ID NO: 385 (Chothia) | HCDR1 | GYSFTSS |
| SEQ ID NO: 386 (Chothia) | HCDR2 | YPGQSD |
| SEQ ID NO: 387 (Chothia) | HCDR3 | VHIIQPPSAWSYNAMDV |
| SEQ ID NO: 388 (Kabat) | LCDR1 | RASQSISTYLN |
| SEQ ID NO: 389 (Kabat) | LCDR2 | GASNLQS |
| SEQ ID NO: 390 (Kabat) | LCDR3 | QQSITELFT |
| SEQ ID NO: 391 (Chothia) | LCDR1 | SQSISTY |
| SEQ ID NO: 392 (Chothia) | LCDR2 | GAS |
| SEQ ID NO: 393 (Chothia) | LCDR3 | SITELF |
| SEQ ID NO: 394 | VH | QVQLVQSGAEVKKPGESLKISCKGSGYSFTSSWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSS |
| SEQ ID NO: 395 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 396 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCACTAGCTCATGGGTTGCTTGGGTGCGCCAGATGCCGGGC |
| SEQ ID NO: 397 | DNA VL | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| | | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 398 | Heavy Chain | QVQLVQSGAEVKKPGESLKISCKGSGYSFTSSWVAWVRQMPGKGLEWMGIIYPGQSDTIYSPSFQ |
| | | GQVTISADKSISTAYLQWSSLKASDTAMYYCARVHIIQPPSAWSYNAMDVWGQGTLVTVSSASTKG |
| | | PSVFPLAPSSKSTSGGTAALGCLVKDYFPEPVTVSWNSGALTSGVHTFPAVLOSSGLYSLSSVVIVPSS |
| | | SLGTQTYICNVNHKPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 399 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 400 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTAGCTCATGGGTTGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 401 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13867 | | |
| SEQ ID NO: 402 (Kabat) | HCDR1 | NYWIA |
| SEQ ID NO: 403 (Kabat) | HCDR2 | IlYPGQSDTIYSPSFQG |
| SEQ ID NO: 404 (Kabat) | HCDR3 | VHIIQPPSAWSYNAMDV |
| SEQ ID NO: 405 (Chothia) | HCDR1 | GYSFSNY |
| SEQ ID NO: 406 (Chothia) | HCDR2 | YPGQSD |
| SEQ ID NO: 407 (Chothia) | HCDR3 | VHIIQPPSAWSYNAMDV |
| SEQ ID NO: 408 (Kabat) | LCDR1 | RASQSISTYLN |
| SEQ ID NO: 409 (Kabat) | LCDR2 | GASNLQS |
| SEQ ID NO: 410 (Kabat) | LCDR3 | QQSITELFT |
| SEQ ID NO: 411 (Chothia) | LCDR1 | SQSISTY |
| SEQ ID NO: 412 (Chothia) | LCDR2 | GAS |
| SEQ ID NO: 413 (Chothia) | LCDR3 | SITELF |
| SEQ ID NO: 414 | VH | QVQLVQSGAEVKKPGESLKISCKGSGYSFSNYWIAWVRQMPGKGLEWMGIIYPGQSDTIYSPSFQG |
| | | QVTISADKSISTAYLQWSSLKASDTAMYYCARVHIIQPPSAWSYNAMDVWGQGTLVTVSS |
| SEQ ID NO: 415 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 416 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 417 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 418 | Heavy Chain | QVQLVQSGAEVKKPGESLKISCKGSGYSFSNYWIAWVRQMPGKGLEWMGIIYPGQSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 419 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 420 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 421 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13868 | | |
| SEQ ID NO: 422 (Kabat) | HCDR1 | NYWIA |
| SEQ ID NO: 423 (Kabat) | HCDR2 | IIYPGLSDTIYSPSFQG |
| SEQ ID NO: 424 (Kabat) | HCDR3 | VHIIQPPSAWSYNAMDV |
| SEQ ID NO: 425 (Chothia) | HCDR1 | GYSFSNY |
| SEQ ID NO: 426 (Chothia) | HCDR2 | YPGLSD |
| SEQ ID NO: 427 (Chothia) | HCDR3 | VHIIQPPSAWSYNAMDV |
| SEQ ID NO: 428 (Kabat) | LCDR1 | RASQSISTYLN |
| SEQ ID NO: 429 (Kabat) | LCDR2 | GASNLQS |
| SEQ ID NO: 430 (Kabat) | LCDR3 | QQSITELFT |
| SEQ ID NO: 431 (Chothia) | LCDR1 | SQSISTY |
| SEQ ID NO: 432 (Chothia) | LCDR2 | GAS |
| SEQ ID NO: 433 (Chothia) | LCDR3 | SITELF |
| SEQ ID NO: 434 | VH | QVQLVQSGAEVKKPGESLKISCKGSGYSFSNYWIAWVRQMPGKGLEWMGIIYPGLSDTIYSPSFQG |
| | | QVTISADKSISTAYLQWSSLKASDTAMYYCARVHIIQPPSAWSYNAMDVWGQGTLVTVSS |
| SEQ ID NO: 435 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 436 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTCTGAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 437 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 438 | Heavy Chain | QVQLVQSGAEVKKPGESLKISCKGSGYSFSNYWIAWVRQMPGKGLEWMGIIYPGLSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 439 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 440 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTCTGAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 441 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13869 | | |
| SEQ ID NO: 442 (Kabat) | HCDR1 | NYWIA |
| SEQ ID NO: 443 (Kabat) | HCDR2 | IIYPGGSDTIYSPSFQG |
| SEQ ID NO: 444 (Kabat) | HCDR3 | VHIIQPPSAWSYNAMDV |
| SEQ ID NO: 445 (Chothia) | HCDR1 | GYSFSNY |
| SEQ ID NO: 446 (Chothia) | HCDR2 | YPGGSD |
| SEQ ID NO: 447 (Chothia) | HCDR3 | VHIIQPPSAWSYNAMDV |
| SEQ ID NO: 448(Kabat) | LCDR1 | RASQSISTYLN |
| SEQ ID NO: 449 (Kabat) | LCDR2 | GASNLQS |
| SEQ ID NO: 450 (Kabat) | LCDR3 | QQSITELFT |
| SEQ ID NO: 451 (Chothia) | LCDR1 | SQSISTY |
| SEQ ID NO: 452 (Chothia) | LCDR2 | GAS |
| SEQ ID NO: 453 (Chothia) | LCDR3 | SITELF |
| SEQ ID NO: 454 | VH | QVQLVQSGAEVKKPGESLKISCKGSGYSFSNYWIAWVRQMPGKGLEWMGIIYPGGSDTIYSPSFQG |
| | | QVTISADKSISTAYLQWSSLKASDTAMYYCARVHIIQPPSAWSYNAMDVWGQGTLVTVSS |
| SEQ ID NO: 455 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 456 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTGGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCA |
| SEQ ID NO: 457 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 458 | Heavy Chain | QVQLVQSGAEVKKPGESLKISCKGSGYSFSNYWIAWVRQMPGKGLEWMGIIYPGGSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 459 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 460 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTGGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCAGC |
| | | CTCCACCAAGGGTCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTGAAT |
| | | CACAAGCCCAGCAACACCAAGGTGGACAAGAGAGTTGAGCCCAAATCTTGTGACAAAACTCAC |
| | | ACATGCCCACCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCAA |
| | | GACAAAGCCGCGGGAGGAGCAGTACAACAGCACGTACCGGGTGGTCAGCGTCCTCACCGTCCT |
| | | GCACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCAAAGGCTT |
| | | CTATCCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| SEQ ID NO: 461 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13870 | | |
| SEQ ID NO: 462 (Kabat) | HCDR1 | NYWIA |
| SEQ ID NO: 463 (Kabat) | HCDR2 | IIYPGTSDTIYSPSFQG |
| SEQ ID NO: 464 (Kabat) | HCDR3 | VHIIQPPSAWSYNAMDV |
| SEQ ID NO: 465 (Chothia) | HCDR1 | GYSFSNY |
| SEQ ID NO: 466 (Chothia) | HCDR2 | YPGTSD |
| SEQ ID NO: 467 (Chothia) | HCDR3 | VHIIQPPSAWSYNAMDV |
| SEQ ID NO: 468 (Kabat) | LCDR1 | RASQSISTYLN |
| SEQ ID NO: 469 (Kabat) | LCDR2 | GASNLQS |
| SEQ ID NO: 470 (Kabat) | LCDR3 | QQSITELFT |
| SEQ ID NO: 471 (Chothia) | LCDR1 | SQSISTY |
| SEQ ID NO: 472 (Chothia) | LCDR2 | GAS |
| SEQ ID NO: 473 (Chothia) | LCDR3 | SITELF |
| SEQ ID NO: 474 | VH | QVQLVQSGAEVKKPGESLKISCKGSGYSFSNYWIAWVRQMPGKGLEWMGIIYPGTSDTIYSPSFQG |
| | | QVTISADKSISTAYLQWSSLKASDTAMYYCARVHIIQPPSAWSYNAMDVWGQGTLVTVSS |
| SEQ ID NO: 475 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 476 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTACCAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 477 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 478 | Heavy Chain | QVQLVQSGAEVKKPGESLKISCKGSGYSFSNYWIAWVRQMPGKGLEWMGIIYPGTSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 479 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 480 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTACCAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 481 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR13871 | | |
| SEQ ID NO: 482 (Kabat) | HCDR1 | NYWIA |
| SEQ ID NO: 483 (Kabat) | HCDR2 | IIYPGSSDTIYSPSFQG |
| SEQ ID NO: 484 (Kabat) | HCDR3 | VHIIQPPSAWSYNAMDV |
| SEQ ID NO: 485 (Chothia) | HCDR1 | GYSFSNY |
| SEQ ID NO: 486 (Chothia) | HCDR2 | YPGSSD |
| SEQ ID NO: 487 (Chothia) | HCDR3 | VHIIQPPSAWSYNAMDV |
| SEQ ID NO: 488 (Kabat) | LCDR1 | RASQSISTYLN |
| SEQ ID NO: 489 (Kabat) | LCDR2 | GASNLQS |
| SEQ ID NO: 490 (Kabat) | LCDR3 | QQSITELFT |
| SEQ ID NO: 491 (Chothia) | LCDR1 | SQSISTY |
| SEQ ID NO: 492 (Chothia) | LCDR2 | GAS |
| SEQ ID NO: 493 (Chothia) | LCDR3 | SITELF |
| SEQ ID NO: 494 | VH | QVQLVQSGAEVKKPGESLKISCKGSGYSFSNYWIAWVRQMPGKGLEWMGIIYPGSSDTIYSPSFQG |
| | | QVTISADKSISTAYLQWSSLKASDTAMYYCARVHIIQPPSAWSYNAMDVWGQGTLVTVSS |
| SEQ ID NO: 495 | VL | DIQMTQSPSSLSASVGDRVTITCRASQSISTYLNWYQQKPGKAPKLLIFGASNLQSGVPSRFSGSGSG |
| | | TDFTLTISSLQPEDFATYYCQQSITELFTFGQGTKVEIK |
| SEQ ID NO: 496 | DNA VH | CAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTAGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCA |
| SEQ ID NO: 497 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 498 | Heavy Chain | QVQLVQSGAEVKKPGESLKISCKGSGYSFSNYWIAWVRQMPGKGLEWMGIIYPGSSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 499 | Light Chain | DIQMTQSPSSLSASVGDRVTITCRASQSISTYLNWYQQKPGKAPKLLIFGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 500 | DNA Heavy | CAGGTGCAATTGGTGCAGAGCGGTGCGGAAGTGAAAAAACCGGGCGAAAGCCTGAAAATTAG |
| | Chain | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTAGCAGCGACACCATCTATAGCCCGAGC |
| | | TTTCAGGGCCAGGTGACCATTAGCGCGGATAAAAGCATCAGCACCGCGTATCTGCAATGGAGC |
| | | AGCCTGAAAGCGAGCGATACCGCGATGTATTATTGCGCGCGTGTTCATATCATCCAGCCGCCGT |
| | | CTGCTTGGTCTTACAACGCTATGGATGTTTGGGGCCAAGGCACCCTGGTGACTGTTAGCTCAGC |
| | | CTCCACCAAGGGTCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCAACGTGAAT |
| | | CACAAGCCCAGCAACACCAAGGTGGACAAGAGAGTTGAGCCCAAATCTTGTGACAAAACTCAC |
| | | ACATGCCCACCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATAATGCCAA |
| | | GACAAAGCCGCGGGAGGAGCAGTACAACAGCACGTACCGGGTGGTCAGCGTCCTCACCGTCCT |
| | | GCACCAGGACTGGCTGAATGGCAAGGAGTACAAGTGCAAGGTCTCCAACAAAGCCCTCCCAGC |
| | | CCCCATCGAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCAAAGGCTT |
| | | CTATCCCAGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCTACAGCAAGCTCACCGTGGACAAG |
| | | AGCAGGTGGCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| SEQ ID NO: 501 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTTCGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR14535 | | |
| SEQ ID NO: 502 (Kabat) | HCDR1 | TTWVA |
| SEQ ID NO: 503 (Kabat) | HCDR2 | IIYPGLSDTIYSPSFQG |
| SEQ ID NO: 504 (Kabat) | HCDR3 | VHIIQPPSAWSYNAMDV |
| SEQ ID NO: 505 (Chothia) | HCDR1 | GYSFTTT |
| SEQ ID NO: 506 (Chothia) | HCDR2 | YPGLSD |
| SEQ ID NO: 507 (Chothia) | HCDR3 | VHIIQPPSAWSYNAMDV |
| SEQ ID NO: 508 (Kabat) | LCDR1 | RASQSISTYLN |
| SEQ ID NO: 509 (Kabat) | LCDR2 | GASNLQS |
| SEQ ID NO: 510 (Kabat) | LCDR3 | QQSITELFT |
| SEQ ID NO: 511 (Chothia) | LCDR1 | SQSISTY |
| SEQ ID NO: 512 (Chothia) | LCDR2 | GAS |
| SEQ ID NO: 513 (Chothia) | LCDR3 | SITELF |
| SEQ ID NO: 514 | VH | EVQLVQSGAEVKKPGESLKISCKGSGYSFTTTWVAWVRQMPGKGLEWMGIIYPGLSDTIYSPSFQG |
| | | QVTISADKSISTAYLQWSSLKASDTAMYYCARVHIIQPPSAWSYNAMDVWGQGTLVTVSS |
| SEQ ID NO: 515 | VL | DIQMTQSPSSLSASVGDRVTITCRASQSISTYLNWYQQKPGKAPKLLIYGASNLQSGVPSRFSGSGSG |
| | | TDFTLTISSLQPEDFATYYCQQSITELFTFGQGTKVEIK |
| SEQ ID NO: 516 | DNA VH | GAGGTGCAATTGGTGCAGAGCGGTGCGGAAGTGAAAAAACCGGGCGAAAGCCTGAAAATTAG |
| | | CTGCAAAGGCTCCGGATATAGCTTCACTACCACTTGGGTTGCTTGGGTGCGCCAGATGCCGGGC |
| | | AAAGGTCTCGAGTGGATGGGCATCATCTACCCGGGTCTGAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 517 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTACGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 518 | Heavy Chain | EVQLVQSGAEVKKPGESLKISCKGSGYSFTTTWVAWVRQMPGKGLEWMGIIYPGLSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 519 | Light Chain | DIQMTQSPSSLSASVGDRVTITCRASQSISTYLNWYQQKPGKAPKLLIYGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 520 | DNA Heavy | GAGGTGCAATTGGTGCAGAGCGGTGCGGAAGTGAAAAAACCGGGCGAAAGCCTGAAAATTAG |
| | Chain | CTGCAAAGGCTCCGGATATAGCTTCACTACCACTTGGGTTGCTTGGGTGCGCCAGATGCCGGGC |
| | | AAAGGTCTCGAGTGGATGGGCATCATCTACCCGGGTCTGAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| SEQ ID NO: 521 | DNA Light | GATATCCAGATGACCCAGAGCCCGAGCAGCCTGAGCGCCAGCGTGGGCGATCGCGTGACCATT |
| | Chain | ACCTGCAGAGCCAGCCAGTCTATTTCTACTTACCTGAACTGGTACCAGCAGAAACCGGGCAAAG |
| | | CGCCGAAACTATTAATCTACGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |
| MOR14536 | | |
| SEQ ID NO: 522 (Kabat) | HCDR1 | NYWIA |
| SEQ ID NO: 523 (Kabat) | HCDR2 | IIYPGQSDTIYSPSFQG |
| SEQ ID NO: 524 (Kabat) | HCDR3 | VHIIQPPSAWSYNAMDV |
| SEQ ID NO: 525 (Chothia) | HCDR1 | GYSFSNY |
| SEQ ID NO: 526 (Chothia) | HCDR2 | YPGQSD |
| SEQ ID NO: 527 (Chothia) | HCDR3 | VHIIQPPSAWSYNAMDV |
| SEQ ID NO: 528 (Kabat) | LCDR1 | RASQSISTYLN |
| SEQ ID NO: 529 (Kabat) | LCDR2 | GASNLQS |
| SEQ ID NO: 530 (Kabat) | LCDR3 | QQSITELFT |
| SEQ ID NO: 531 (Chothia) | LCDR1 | SQSISTY |
| SEQ ID NO: 532 (Chothia) | LCDR2 | GAS |
| SEQ ID NO: 533 (Chothia) | LCDR3 | SITELF |
| SEQ ID NO: 534 | VH | EVOIVQSGAEVKKPGESLKISCKGSGYSFSNYWIAWVRQMPGKGLEWMGIIYPGQSDTIYSPSFQG |
| | | QVTISADKSISTAYLQWSSLKASDTAMYYCARVHIIQPPSAWSYNAMDVWGQGTLVTVSS |
| SEQ ID NO: 535 | VL | DIQMTQSPSSLSASVGDRVTITCRASQSISTYLNWYQQKPGKAPKLLIYGASNLQSGVPSRFSGSGSG |
| | | TDFTLTISSLQPEDFATYYCQQSITELFTFGQGTKVEIK |
| SEQ ID NO: 536 | DNA VH | GAGGTGCAATTGGTGCAGAGCGGTGCGGAAGTGAAAAAACCGGGCGAAAGCCTGAAAATTAG |
| | | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCA |
| SEQ ID NO: 537 | DNA VL | GATATCCAGATGACCCAGAGCCCGAGCAGCCTGAGCGCCAGCGTGGGCGATCGCGTGACCATT |
| | | ACCTGCAGAGCCAGCCAGTCTATTTCTACTTACCTGAACTGGTACCAGCAGAAACCGGGCAAAG |
| | | CGCCGAAACTATTAATCTACGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| SEQ ID NO: 538 | Heavy Chain | EVOIVQSGAEVKKPGESLKISCKGSGYSFSNYWIAWVRQMPGKGLEWMGIIYPGQSDTIYSPSFQG |
| | | QVTISADKSISTAYLQWSSLKASDTAMYYCARVHIIQPPSAWSYNAMDVWGQGTLVTVSSASTKGP |
| | | SVFPLAPSSKSTSGGTAALGCLVKDYFPEPVTVSWNSGALTSGVHTFPAVLQSSGLYSLSSVVTVPSSS |
| | | LGTQTYICNVNHKPSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| SEQ ID NO: 539 | Light Chain | DIQMTQSPSSLSASVGDRVTITCRASQSISTYLNWYQQKPGKAPKLLIYGASNLQSGVPSRFSGSGSG |
| | | TDFTLTISSLOPEDFATYYCQQSITELFTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPV |
| | | TKSFNRGEC |
| SEQ ID NO: 540 | DNA Heavy | GAGGTGCAATTGGTGCAGAGCGGTGCGGAAGTGAAAAAACCGGGCGAAAGCCTGAAAATTAG |
| | Chain + Leader | CTGCAAAGGCTCCGGATATAGCTTCTCTAACTACTGGATCGCTTGGGTGCGCCAGATGCCGGGC |
| | | AAAGGTCTCGAGTGGATGGGCATCATCTACCCGGGTCAAAGCGACACCATCTATAGCCCGAGCT |
| | | TTCAGGGCCAGGTGACCATTAGCGCGGATAAAAGCATCAGCACCGCGTATCTGCAATGGAGCA |
| | | GCCTGAAAGCGAGCGATACCGCGATGTATTATTGCGCGCGTGTTCATATCATCCAGCCGCCGTC |
| | | TGCTTGGTCTTACAACGCTATGGATGTTTGGGGCCAAGGCACCCTGGTGACTGTTAGCTCAGCC |
| | | TCCACCAAGGGTCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTGGAACTCAG |
| | | GCGCCCTGACCAGCGGCGTGCACACCTTCCCGGCTGTCCTACAGTCCTCAGGACTCTACTCCCTC |
| | | AGCAGCGTGGTGACCGTGCCCTCCAGCAGCTTGGGCACCCAGACCTACATCTGCAACGTGAATC |
| | | ACAAGCCCAGCAACACCAAGGTGGACAAGAGAGTTGAGCCCAAATCTTGTGACAAAACTCACA |
| | | CATGCCCACCGTGCCCAGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATGCCAAGA |
| | | CAAAGCCGCGGGAGGAGCAGTACAACAGCACGTACCGGGTGGTCAGCGTCCTCACCGTCCTGC |
| | | ACCAGGACTGGCTGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAGGTGTACACCCTGC |
| | | CCCCATCCCGGGAGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGTGGACAAGA |
| | | GCAGGTGGCAGCAGGGGAACGTCTTCTCATGCTCCGTGATGCATGAGGCTCTGCACAACCACTA |
| | | CACGCAGAAGAGCCTCTCCCTGTCTCCGGGTAAA |
| | | GATATCCAGATGACCCAGAGCCCGAGCAGCCTGAGCGCCAGCGTGGGCGATCGCGTGACCATT |
| SEQ ID NO: 541 | DNA Light | ACCTGCAGAGCCAGCCAGTCTATTTCTACTTACCTGAACTGGTACCAGCAGAAACCGGGCAAAG |
| | Chain + Leader | CGCCGAAACTATTAATCTACGGTGCTTCTAACCTGCAAAGCGGCGTGCCGAGCCGCTTTAGCGG |
| | | CAGCGGATCCGGCACCGATTTCACCCTGACCATTAGCTCTCTGCAACCGGAAGACTTTGCGACCT |
| | | ATTATTGCCAGCAGTCTATCACTGAACTGTTCACCTTTGGCCAGGGCACGAAAGTTGAAATTAAA |
| | | CGTACGGTGGCCGCTCCCAGCGTGTTCATCTTCCCCCCCAGCGACGAGCAGCTGAAGAGCGGCA |
| | | CCGCCAGCGTGGTGTGCCTGCTGAACAACTTCTACCCCCGGGAGGCCAAGGTGCAGTGGAAGG |
| | | TGGACAACGCCCTGCAGAGCGGCAACAGCCAGGAAAGCGTCACCGAGCAGGACAGCAAGGAC |
| | | TCCACCTACAGCCTGAGCAGCACCCTGACCCTGAGCAAGGCCGACTACGAGAAGCACAAGGTG |
| | | TACGCCTGCGAGGTGACCCACCAGGGCCTGTCCAGCCCCGTGACCAAGAGCTTCAACCGGGGC |
| | | GAGTGT |

The present invention provides antibodies that specifically bind a HER3 protein (e.g., human and/or cynomologus HER3), the antibodies comprising a VH domain having an amino acid sequence of SEQ ID NO: 14, 34, 54, 74, 94, 114, 134, 154, 174, 194, 214, 234, 254, 274, 294, 314, 334, 354, 374, 394, 414, 434, 454, 474, 494, 514, and 524. The present invention provides antibodies that specifically bind a HER3 protein (e.g., human and/or cynomologus HER3), the antibodies comprising a VL domain having an amino acid sequence of SEQ ID NO: 15, 35, 55, 75, 95, 115, 135, 155, 175, 195, 215, 235, 255, 275, 295, 315, 335, 355, 375, 395, 415, 435, 455, 475, 495, 515, and 535. The present invention also provides antibodies that specifically bind to a HER3 protein (e.g., human and/or cynomologus HER3), said antibodies comprising a VH CDR having an amino acid sequence of any one of the VH CDRs listed in Table 1. In particular, the invention provides antibodies that specifically bind to a HER3 protein (e.g., human and/or cynomologus HER3), said antibodies comprising (or alternatively, consisting of) one, two, three, four, five or more VH CDRs having an amino acid sequence of any of the VH CDRs listed in Table 1.

Other antibodies of the invention include amino acids that have been mutated, yet have at least 50%, 60%, 70%, 80%, 90%, 95%, 96%, 97%, 98% or 99% identity in the CDR regions with the CDR regions depicted in the sequences described in Table 1. In some embodiments, it includes mutant amino acid sequences wherein no more than 1, 2, 3, 4 or 5 amino acids have been mutated in the CDR regions when compared with the CDR regions depicted in the sequence described Table 1, while still maintaining their specificity for the original antibody's epitope

Other antibodies of the invention include amino acids that have been mutated, yet have at least 50%, 60%, 70%, 80%, 90%, 95%, 96%, 97%, 98% or 99% identity in the framework regions with the framework regions depicted in the sequences described in Table 1. In some embodiments, it includes mutant amino acid sequences wherein no more than 1, 2, 3, 4, 5, 6, or 7 amino acids have been mutated in the framework regions when compared with the framework regions depicted in the sequence described Table 1, while still maintaining their specificity for the original antibody's epitope. The present invention also provides nucleic acid sequences that encode VH, VL, the full length heavy chain, and the full length light chain of the antibodies that specifically bind to a HER3 protein (e.g., human and/or cynomologus HER3).

Other antibodies of the invention include those where the amino acids or nucleic acids encoding the amino acids have been mutated, yet have at least 50%, 60%, 70%, 80%, 90%, 95%, 96%, 97%, 98% or 99% identity to the sequences described in Table 1. In some embodiments, it include mutant amino acid sequences wherein no more than 1, 2, 3, 4 or 5 amino acids have been mutated in the variable regions when compared with the variable regions depicted in the sequence described in Table 1, while retaining substantially the same therapeutic activity.

Since each of these antibodies or fragments thereof can bind to HER3, the VH, VL, full length light chain, and full length heavy chain sequences (amino acid sequences and the nucleotide sequences encoding the amino acid sequences) can be “mixed and matched” to create other HER3 antibodies of the invention. Such “mixed and matched” HER3 antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

Accordingly, in one aspect, the invention provides an isolated monoclonal antibody or fragment thereof having: VH comprising an amino acid sequence selected from the group consisting of SEQ ID NO: 14, 34, 54, 74, 94, 114, 134, 154, 174, 194, 214, 234, 254, 274, 294, 314, 334, 354, 374, 394, 414, 434, 454, 474, 494, 514, and 524; and VL comprising an amino acid sequence selected from the group consisting of SEQ ID NOs: 15, 35, 55, 75, 95, 115, 135, 155, 175, 195, 215, 235, 255, 275, 295, 315, 335, 355, 375, 395, 415, 435, 455, 475, 495, 515, and 535; wherein the antibody specifically binds to HER3 (e.g., human and/or cynomologus).

In a specific embodiment, an antibody that binds to HER3 comprises a VH of SEQ ID NO: 14 and VL of SEQ ID NO: 15. In a specific embodiment, an antibody that binds to HER3 comprises a VH of SEQ ID NO: 34 and VL of SEQ ID NO: 35. In a specific embodiment, an antibody that binds to HER3 comprises a VH of SEQ ID NO: 54 and VL of SEQ ID NO: 55. In a specific embodiment, an antibody that binds to HER3 comprises a SEQ ID NO: 74 and VL of SEQ ID NO: 75. In a specific embodiment, an antibody that binds to HER3 comprises a VH of SEQ ID NO: 94 and VL of SEQ ID NO: 95. In a specific embodiment, an antibody that binds to HER3 comprises a VH of SEQ ID NO: 114 and VL of SEQ ID NO: 115. In a specific embodiment, an antibody that binds to HER3 comprises a VH of SEQ ID NO: 134 and VL of SEQ ID NO: 135. In a specific embodiment, an antibody that binds to HER3 comprises a VH of SEQ ID NO: 154 and VL of SEQ ID NO: 155. In a specific embodiment, an antibody that binds to HER3 comprises a VH of SEQ ID NO: 174 and VL of SEQ ID NO: 175. In a specific embodiment, an antibody that binds to HER3 comprises a VH of SEQ ID NO: 194 and VL of SEQ ID NO: 195. In a specific embodiment, an antibody that binds to HER3 comprises a VH of SEQ ID NO: 214 and VL of SEQ ID NO: 215. In a specific embodiment, an antibody that binds to HER3 comprises a VH of SEQ ID NO: 234 and VL of SEQ ID NO: 235. In a specific embodiment, an antibody that binds to HER3 comprises a VH of SEQ ID NO: 254 and VL of SEQ ID NO: 255. In a specific embodiment, an antibody that binds to HER3 comprises a VH of SEQ ID NO: 274 and VL of SEQ ID NO: 275. In a specific embodiment, an antibody that binds to HER3 comprises a VH of SEQ ID NO: 294 and VL of SEQ ID NO: 295. In a specific embodiment, an antibody that binds to HER3 comprises a VH of SEQ ID NO: 314 and VL of SEQ ID NO: 315. In a specific embodiment, an antibody that binds to HER3 comprises a VH of SEQ ID NO: 334 and VL of SEQ ID NO: 335. In a specific embodiment, an antibody that binds to HER3 comprises a VH of SEQ ID NO: 354 and VL of SEQ ID NO: 355. In a specific embodiment, an antibody that binds to HER3 comprises a VH of SEQ ID NO: 374 and VL of SEQ ID NO: 375. In a specific embodiment, an antibody that binds to HER3 comprises a VH of SEQ ID NO: 394 and VL of SEQ ID NO: 395. In a specific embodiment, an antibody that binds to HER3 comprises a VH of SEQ ID NO: 414 and VL of SEQ ID NO: 415. In a specific embodiment, an antibody that binds to HER3 comprises a VH of SEQ ID NO: 434 and VL of SEQ ID NO: 435. In a specific embodiment, an antibody that binds to HER3 comprises a VH of SEQ ID NO: 454 and VL of SEQ ID NO: 455. In a specific embodiment, an antibody that binds to HER3 comprises a VH of SEQ ID NO: 474 and VL of SEQ ID NO: 475. In a specific embodiment, an antibody that binds to HER3 comprises a VH of SEQ ID NO: 494 and VL of SEQ ID NO: 495. In a specific embodiment, an antibody that binds to HER3 comprises a VH of SEQ ID NO: 514 and VL of SEQ ID NO: 515. In a specific embodiment, an antibody that binds to HER3 comprises a VH of SEQ ID NO: 534 and VL of SEQ ID NO: 535.

In another aspect, the present invention provides HER3 antibodies that comprise the heavy chain and light chain CDR1s, CDR2s and CDR3s as described in Table 1, or combinations thereof. The CDR regions are delineated using the Kabat system (Kabat et al., (1991) Sequences of Proteins of Immunological Interest, Fifth Edition, U.S. Department of Health and Human Services, NIH Publication No. 91-3242; Chothia et al., (1987) J. Mol. Biol. 196:901-917; Chothia et al., (1989) Nature 342: 877-883; and Al-Lazikani et al., (1997) J. Mol. Biol. 273, 927-948). Accordingly, in one embodiment, the antibody or fragment thereof comprises a heavy chain variable region antibody sequence having a CDR1 sequence selected from the group consisting of SEQ ID NOs: 2, 22, 42, 62, 82, 102, 122, 142, 162, 182, 202, 222, 242, 262, 282, 302, 322, 342, 362, 382, 402, 422, 442, 462, 482, 502, and 522; a CDR2 sequence selected from the group consisting of SEQ ID NOs: 3, 23, 43, 63, 83, 103, 123, 143, 163, 183, 203, 223, 243, 263, 283, 303, 323, 343, 363, 383, 403, 423, 443, 463, 483, 503, and 523; and/or a CDR3 sequence selected from the group consisting of SEQ ID NOs: 4, 24, 44, 64, 84, 104, 124, 144, 164, 184, 204, 224, 244, 264, 284, 304, 324, 344, 364, 384, 404, 424, 444, 464, 484, 504, and 524; and a light chain variable region antibody sequence having a CDR1 sequence selected from the group consisting of SEQ ID NOs: 8, 28, 48, 68, 88, 108, 128, 148, 168, 188, 208, 228, 248, 268, 288, 308, 328, 348, 368, 388, 408, 428, 448, 468, 488, 508, and 528; a CDR2 sequence selected from the group consisting of SEQ ID NOs: 9, 29, 49, 69, 89, 109, 129, 149, 169, 189, 209, 229, 249, 269, 289, 309, 329, 349, 369, 389, 409, 429, 449, 469, 489, 509, and 529; and/or a CDR3 sequence selected from the group consisting of SEQ ID NOs: 10, 30, 50, 70, 90, 110, 130, 150, 170, 190, 210, 230, 250, 270, 290, 310, 330, 350, 370, 390, 410, 430, 450, 470, 490, 510, and 530, wherein the antibody or fragment thereof binds to domain 2 of HER3.

In a specific embodiment, an antibody that binds to HER3 comprises a heavy chain variable region CDR1 of SEQ ID NO: 502; a CDR2 of SEQ ID NO: 503; a CDR3 of SEQ ID NO: 504; a light chain variable region CDR1 of SEQ ID NO: 508; a CDR2 of SEQ ID NO: 509; and a CDR3 of SEQ ID NO: 510.

In a specific embodiment, an antibody that binds to HER3 comprises a heavy chain variable region CDR1 of SEQ ID NO: 522; a CDR2 of SEQ ID NO: 523; a CDR3 of SEQ ID NO: 524; a light chain variable region CDR1 of SEQ ID NO: 528; a CDR2 of SEQ ID NO: 529; and a CDR3 of SEQ ID NO: 530.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 50%, 60%, 70%, 80%, 90%, 95%, 96%, 97%, 98% or 99% identical in amino acid sequence to the amino acid sequence encoded by the germline immunoglobulin gene. Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene.

The antibodies disclosed herein can be derivatives of single chain antibodies, diabodies, domain antibodies, nanobodies, and unibodies. A “single-chain antibody” (scFv) consists of a single polypeptide chain comprising a VL domain linked to a VH domain, wherein VL domain and VH domain are paired to form a monovalent molecule. Single chain antibody can be prepared according to method known in the art (see, for example, Bird et al., (1988) Science 242:423-426 and Huston et al., (1988) Proc. Natl. Acad. Sci. USA 85:5879-5883). A “disbud” consists of two chains, each chain comprising a heavy chain variable region connected to a light chain variable region on the same polypeptide chain connected by a short peptide linker, wherein the two regions on the same chain do not pair with each other but with complementary domains on the other chain to form a bispecific molecule. Methods of preparing diabodies are known in the art (See, e.g., Holliger et al., (1993) Proc. Natl. Acad. Sci. USA 90:6444-6448, and Poljak et al., (1994) Structure 2:1121-1123). Domain antibodies (dAbs) are small functional binding units of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640; WO05/035572, WO04/101790, WO04/081026, WO04/058821, WO04/003019 and WO03/002609. Nanobodies are derived from the heavy chains of an antibody. A nanobody typically comprises a single variable domain and two constant domains (CH2 and CH3) and retains antigen-binding capacity of the original antibody. Nanobodies can be prepared by methods known in the art (See e.g., U.S. Pat. No. 6,765,087, U.S. Pat. No. 6,838,254, WO 06/079372). Unibodies consist of one light chain and one heavy chain of a IgG4 antibody. Unibodies may be made by the removal of the hinge region of IgG4 antibodies. Further details of unibodies and methods of preparing them may be found in WO2007/059782.

### Homologous Antibodies

In yet another embodiment, the present invention provides an antibody or fragment thereof comprising amino acid sequences that are homologous to the sequences described in Table 1, and said antibody binds to a HER3 protein (e.g., human and/or cynomologus HER3), and retains the desired functional properties of those antibodies described in Table 1.

For example, the invention provides an isolated monoclonal antibody (or a functional fragment thereof) comprising a heavy chain variable region and a light chain variable region, wherein the heavy chain variable region comprises an amino acid sequence that is at least 80%, 90%, 95%, 96%, 97%, 98% or 99% identical to an amino acid sequence selected from the group consisting of SEQ ID NOs: 14, 34, 54, 74, 94, 114, 134, 154, 174, 194, 214, 234, 254, 274, 294, 314, 334, 354, 374, 394, 414, 434, 454, 474, 494, 514, and 524; the light chain variable region comprises an amino acid sequence that is at least 80%, 90%, 95%, 96%, 97%, 98% or 99% identical to an amino acid sequence selected from the group consisting of SEQ ID NOs: 14, 34, 54, 74, 94, 114, 134, 154, 174, 194, 214, 234, 254, 274, 294, 314, 334, 354, 374, 394, 414, 434, 454, 474, 494, 514, and 524; the antibody binds to HER3 (e.g., human and/or cynomologus HER3) and inhibits the signaling activity of HER3, which can be measured in a phosphorylation assay or other measure of HER signaling (e.g., phospho-HER3 assays, phospho-Akt assays, cell proliferation, and ligand blocking assays as described in the Examples). Also includes within the scope of the invention are variable heavy and light chain parental nucleotide sequences; and full length heavy and light chain sequences optimized for expression in a mammalian cell. Other antibodies of the invention include amino acids or nucleic acids that have been mutated, yet have at least 60, 70, 80, 90, 95, 98, or 99% percent identity to the sequences described above. In some embodiments, it include mutant amino acid sequences wherein no more than 1, 2, 3, 4 or 5 amino acids have been mutated by amino acid deletion, insertion or substitution in the variable regions when compared with the variable regions depicted in the sequence described above.

In other embodiments, the VH and/or VL amino acid sequences may be 50%, 60%, 70%, 80%, 90%, 95%, 96%, 97%, 98% or 99% identical to the sequences set forth in Table 1. In other embodiments, the VH and/or VL amino acid sequences may be identical except an amino acid substitution in no more than 1, 2, 3, 4 or 5 amino acid position. An antibody having VH and VL regions having high (i.e., 80% or greater) identity to the VH and VL regions of the antibodies described in Table 1 can be obtained by mutagenesis (e.g., site-directed or PCR-mediated mutagenesis), followed by testing of the encoded altered antibody for retained function using the functional assays described herein.

In other embodiments, the variable regions of heavy chain and/or light chain nucleotide sequences may be 60%, 70%, 80%, 90%, 95%, 96%, 97%, 98% or 99% identical to the sequences set forth above.

As used herein, “percent identity” between the two sequences is a function of the number of identical positions shared by the sequences (i.e., % identity equals number of identical positions/total number of positions×100), taking into account the number of gaps, and the length of each gap, which needs to be introduced for optimal alignment of the two sequences.

The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identifies related sequences. For example, such searches can be performed using the BLAST program (version 2.0) of Altschul et al., (1990) J. Mol. Biol. 215:403-10.

Antibodies with Conservative Modifications

In certain embodiments, an antibody of the invention has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the HER3 antibodies of the invention.

Accordingly, the invention provides an isolated HER3 monoclonal antibody, or a fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 2, 22, 42, 62, 82, 102, 122, 142, 162, 182, 202, 222, 242, 262, 282, 302, 322, 342, 362, 382, 402, 422, 442, 462, 482, 502, and 522, and conservative modifications thereof; the heavy chain variable region CDR2 amino acid sequences are selected from the group consisting of SEQ ID NOs: 3, 23, 43, 63, 83, 103, 123, 143, 163, 183, 203, 223, 243, 263, 283, 303, 323, 343, 363, 383, 403, 423, 443, 463, 483, 503, and 523 and conservative modifications thereof; the heavy chain variable region CDR3 amino acid sequences are selected from the group consisting of SEQ ID NOs: 4, 24, 44, 64, 84, 104, 124, 144, 164, 184, 204, 224, 244, 264, 284, 304, 324, 344, 364, 384, 404, 424, 444, 464, 484, 504, and 524 and conservative modifications thereof; the light chain variable regions CDR1 amino acid sequences are selected from the group consisting of SEQ ID NOs: 8, 28, 48, 68, 88, 108, 128, 148, 168, 188, 208, 228, 248, 268, 288, 308, 328, 348, 368, 388, 408, 428, 448, 468, 488, 508, and 528 and conservative modifications thereof; the light chain variable regions CDR2 amino acid sequences are selected from the group consisting of SEQ ID NOs: 9, 29, 49, 69, 89, 109, 129, 149, 169, 189, 209, 229, 249, 269, 289, 309, 329, 349, 369, 389, 409, 429, 449, 469, 489, 509, and 529, and conservative modifications thereof; the light chain variable regions of CDR3 amino acid sequences are selected from the group consisting of SEQ ID NOs: 10, 30, 50, 70, 90, 110, 130, 150, 170, 190, 210, 230, 250, 270, 290, 310, 330, 350, 370, 390, 410, 430, 450, 470, 490, 510, and 530, and conservative modifications thereof; the antibody or fragment thereof specifically binds to HER3, and inhibits HER3 activity by inhibiting a HER3 signaling pathway, which can be measured in a phosphorylation assay or other measure of HER signaling (e.g., phospho-HER3 assays, phospho-Akt assays, cell proliferation, and ligand blocking assays as described in the Examples).

Antibodies that Bind to the Same Epitope

The present invention provides antibodies that interacts with (e.g., by binding, steric hindrance, stabilizing/destabilizing, spatial distribution) the same epitope as do the HER3 antibodies described in Table 1. Additional antibodies can therefore be identified based on their ability to cross-compete (e.g., to competitively inhibit the binding of, in a statistically significant manner) with other antibodies of the invention in HER3 binding assays. The ability of a test antibody to inhibit the binding of antibodies of the present invention to a HER3 protein (e.g., human and/or cynomologus HER3) demonstrates that the test antibody can compete with that antibody for binding to HER3; such an antibody may, according to non-limiting theory, bind to the same or a related (e.g., a structurally similar or spatially proximal) epitope on the HER3 protein as the antibody with which it competes. In a certain embodiment, the antibody that binds to the same epitope on HER3 as the antibodies of the present invention is a human monoclonal antibody. Such human monoclonal antibodies can be prepared and isolated as described herein.

In one embodiment, the antibody or fragments thereof binds to domain 2 of HER3 to hold the HER3 in conformation which prevents exposure of an dimerization loop present within domain 2. This prevents heterodimerizaton with other family members, such as HER1, HER2, and HER4. The antibodies of fragments thereof inhibit both ligand dependent and ligand-independent HER3 signal transduction.

In another embodiment, the antibody or fragment thereof binds to domain 2 of HER3 without blocking the concurrent binding of a HER3 ligand such as neuregulin. While not required to provide a theory, it is feasible that the antibody or fragment thereof binding to domain 2 of HER3, holds HER3 a conformation that does not block the ligand binding site on HER3. Thus a HER3 ligand (e.g., neuregulin) is able to bind to HER3 at the same time as the antibody or fragment thereof.

The antibodies of the invention or fragments thereof inhibit both ligand dependent and independent activation of HER3 without preventing ligand binding. This is considered advantageous for the following reasons:

(i) The therapeutic antibody would have clinical utility in a broad spectrum of tumors than an antibody which targeted a single mechanism of HER3 activation (i.e. ligand dependent or ligand independent) since distinct tumor types are driven by each mechanism.

(ii) The therapeutic antibody would be efficacious in tumor types where both mechanisms of HER3 activation are simultaneously involved. An antibody targeting a single mechanism of HER3 activation (i.e. ligand dependent or ligand independent) would display little or no efficacy in these tumor types

(iii) The efficacy of an antibody which inhibits ligand dependent activation of HER3 without preventing ligand binding would be less likely to be adversely affected by increasing concentrations of ligand. This would translate to either increased efficacy in a tumor type driven by very high concentrations of HER3 ligand or a reduced drug resistance liability where resistance is mediated by up-regulation of HER3 ligands.

(iv) An antibody which inhibits HER3 activation by stabilizing the inactive form would be less prone to drug resistance driven by alternative mechanisms of HER3 activation.

Consequently, the antibodies of the invention may be used to treat conditions where existing therapeutic antibodies are clinically ineffective.

### Engineered and Modified Antibodies

An antibody of the invention further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the invention pertains to an isolated HER3 monoclonal antibody, or fragment thereof, comprising a heavy chain variable region comprising CDR1 sequences having an amino acid sequence selected from the group consisting of SEQ ID NOs: 2, 22, 42, 62, 82, 102, 122, 142, 162, 182, 202, 222, 242, 262, 282, 302, 322, 342, 362, 382, 402, 422, 442, 462, 482, 502, and 522; CDR2 sequences having an amino acid sequence selected from the group consisting of SEQ ID NOs: 3, 23, 43, 63, 83, 103, 123, 143, 163, 183, 203, 223, 243, 263, 283, 303, 323, 343, 363, 383, 403, 423, 443, 463, 483, 503, and 523; CDR3 sequences having an amino acid sequence selected from the group consisting of SEQ ID NOs: 4, 24, 44, 64, 84, 104, 124, 144, 164, 184, 204, 224, 244, 264, 284, 304, 324, 344, 364, 384, 404, 424, 444, 464, 484, 504, and 524, respectively; and a light chain variable region having CDR1 sequences having an amino acid sequence selected from the group consisting of SEQ ID NOs: 8, 28, 48, 68, 88, 108, 128, 148, 168, 188, 208, 228, 248, 268, 288, 308, 328, 348, 368, 388, 408, 428, 448, 468, 488, 508, and 528; CDR2 sequences having an amino acid sequence selected from the group consisting of SEQ ID NOs: 9, 29, 49, 69, 89, 109, 129, 149, 169, 189, 209, 229, 249, 269, 289, 309, 329, 349, 369, 389, 409, 429, 449, 469, 489, 509, and 529; and CDR3 sequences consisting of an amino acid sequence selected from the group consisting of SEQ ID NOs: 10, 30, 50, 70, 90, 110, 130, 150, 170, 190, 210, 230, 250, 270, 290, 310, 330, 350, 370, 390, 410, 430, 450, 470, 490, 510, and 530, respectively. Thus, such antibodies contain the VH and VL CDR sequences of monoclonal antibodies, yet may contain different framework sequences from these antibodies. Such framework sequences can be obtained from public DNA databases or published references that include germline antibody gene sequences. For example, germline DNA sequences for human heavy and light chain variable region genes can be found in the “Vase” human germline sequence database (available on the Internet at www.mrc-cpe.cam.ac.uk/vbase), as well as in Kabat et al., (1991) Sequences of Proteins of Immunological Interest, Fifth Edition, U.S. Department of Health and Human Services, NIH Publication No. 91-3242; Chothia et al., (1987) J. Mol. Biol. 196:901-917; Chothia et al., (1989) Nature 342:877-883; and Al-Lazikani et al., (1997) J. Mol. Biol. 273:927-948; Tomlinson et al., (1992) J. fol. Biol. 227:776-798; and Cox et al., (1994) Eur. J Immunol. 24:827-836; the contents of each of which are expressly incorporated herein by reference.

An example of framework sequences for use in the antibodies of the invention are those that are structurally similar to the framework sequences used by selected antibodies of the invention, e.g., consensus sequences and/or framework sequences used by monoclonal antibodies of the invention. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Accordingly, in another embodiment, the invention provides isolated HER3 monoclonal antibodies, or fragment thereof, consisting of a heavy chain variable region having: a VH CDR1 region consisting of an amino acid sequence selected from the group having SEQ ID NOs: 2, 22, 42, 62, 82, 102, 122, 142, 162, 182, 202, 222, 242, 262, 282, 302, 322, 342, 362, 382, 402, 422, 442, 462, 482, 502, and 522 or an amino acid sequence having one, two, three, four or five amino acid substitutions, deletions or additions as compared to SEQ ID NOs: 2, 22, 42, 62, 82, 102, 122, 142, 162, 182, 202, 222, 242, 262, 282, 302, 322, 342, 362, 382, 402, 422, 442, 462, 482, 502, and 522; a VH CDR2 region having an amino acid sequence selected from the group consisting of SEQ ID NOs: 3, 23, 43, 63, 83, 103, 123, 143, 163, 183, 203, 223, 243, 263, 283, 303, 323, 343, 363, 383, 403, 423, 443, 463, 483, 503, and 523 or an amino acid sequence having one, two, three, four or five amino acid substitutions, deletions or additions as compared to SEQ ID NOs: 3, 23, 43, 63, 83, 103, 123, 143, 163, 183, 203, 223, 243, 263, 283, 303, 323, 343, 363, 383, 403, 423, 443, 463, 483, 503, and 523; a VH CDR3 region having an amino acid sequence selected from the group consisting of SEQ ID NOs: 4, 24, 44, 64, 84, 104, 124, 144, 164, 184, 204, 224, 244, 264, 284, 304, 324, 344, 364, 384, 404, 424, 444, 464, 484, 504, and 524, or an amino acid sequence having one, two, three, four or five amino acid substitutions, deletions or additions as compared to SEQ ID NOs: 4, 24, 44, 64, 84, 104, 124, 144, 164, 184, 204, 224, 244, 264, 284, 304, 324, 344, 364, 384, 404, 424, 444, 464, 484, 504, and 524; a VL CDR1 region having an amino acid sequence selected from the group consisting of SEQ ID NOs: 8, 28, 48, 68, 88, 108, 128, 148, 168, 188, 208, 228, 248, 268, 288, 308, 328, 348, 368, 388, 408, 428, 448, 468, 488, 508, and 528, or an amino acid sequence having one, two, three, four or five amino acid substitutions, deletions or additions as compared to SEQ ID NOs: 8, 28, 48, 68, 88, 108, 128, 148, 168, 188, 208, 228, 248, 268, 288, 308, 328, 348, 368, 388, 408, 428, 448, 468, 488, 508, and 528; a VL CDR2 region having an amino acid sequence selected from the group consisting of SEQ ID NOs: 9, 29, 49, 69, 89, 109, 129, 149, 169, 189, 209, 229, 249, 269, 289, 309, 329, 349, 369, 389, 409, 429, 449, 469, 489, 509, and 529, or an amino acid sequence having one, two, three, four or five amino acid substitutions, deletions or additions as compared to SEQ ID NOs: 9, 29, 49, 69, 89, 109, 129, 149, 169, 189, 209, 229, 249, 269, 289, 309, 329, 349, 369, 389, 409, 429, 449, 469, 489, 509, and 529; and a VL CDR3 region having an amino acid sequence selected from the group consisting of SEQ ID NOs: 10, 30, 50, 70, 90, 110, 130, 150, 170, 190, 210, 230, 250, 270, 290, 310, 330, 350, 370, 390, 410, 430, 450, 470, 490, 510, and 530, or an amino acid sequence having one, two, three, four or five amino acid substitutions, deletions or additions as compared to SEQ ID NOs: 10, 30, 50, 70, 90, 110, 130, 150, 170, 190, 210, 230, 250, 270, 290, 310, 330, 350, 370, 390, 410, 430, 450, 470, 490, 510, and 530.

Grafting Antibody Fragments into Alternative Frameworks or Scaffolds

A wide variety of antibody/immunoglobulin frameworks or scaffolds can be employed so long as the resulting polypeptide includes at least one binding region which specifically binds to HER3. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In one aspect, the invention pertains to generating non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target HER3 protein (e.g., human and/or cynomologus HER3). Known non-immunoglobulin frameworks or scaffolds include, but are not limited to, fibronectin (Compound Therapeutics, Inc., Waltham, Mass.), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany)

The fibronectin scaffolds are based on fibronectin type III domain (e.g., the tenth module of the fibronectin type III (¹⁰Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as HER3. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

In some embodiments, the Fabs are converted to silent IgG1 format by changing the Fc region. For example, antibodies in Table 1 can be converted to IgG format.

### Human or Humanized Antibodies

The present invention provides fully human antibodies that specifically bind to a HER3 protein (e.g., human and/or cynomologus/mouse/rat HER3). Compared to the chimeric or humanized antibodies, the human HER3 antibodies or fragments thereof, have further reduced antigenicity when administered to human subjects.

Human HER3 antibodies or fragments thereof can be generated using methods that are known in the art. For example, the humaneering technology used to converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

Using one of the mouse or chimeric HER3 antibodies or fragments thereof described above as the reference antibody, this method can be readily employed to generate human antibodies that bind to human HER3 with the same binding specificity and the same or better binding affinity. In addition, such human HER3 antibodies or fragments thereof can also be commercially obtained from companies which customarily produce human antibodies, e.g., KaloBios, Inc. (Mountain View, Calif.).

### Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans et al., (2004) J Biol Chem 279:1256-1261; Dumoulin et al., (2003) Nature 424:783-788; Pleschberger et al., (2003) Bioconjugate Chem 14:440-448; Cortez-Retamozo et al., (2002) Int J Cancer 89:456-62; and Lauwereys et al., (1998) EMBO J 17:3512-3520. Engineered libraries of camelid antibodies and antibody fragments are commercially available, for example, from Ablynx, Ghent, Belgium. (e.g., US20060115470; Domantis (US20070065440, US20090148434). As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. Further, these molecules can be fully expressed in prokaryotic cells such as E. coli and are expressed as fusion proteins with bacteriophage and are functional.

Accordingly, a feature of the present invention is a camelid antibody or nanobody having high affinity for HER3. In certain embodiments herein, the camelid antibody or nanobody is naturally produced in the camelid animal, i.e., is produced by the camelid following immunization with HER3 or a peptide fragment thereof, using techniques described herein for other antibodies. Alternatively, the HER3-binding camelid nanobody is engineered, i.e., produced by selection for example from a library of phage displaying appropriately mutagenized camelid nanobody proteins using panning procedures with HER3 as a target as described in the examples herein. Engineered nanobodies can further be customized by genetic engineering to have a half life in a recipient subject of from 45 minutes to two weeks. In a specific embodiment, the camelid antibody or nanobody is obtained by grafting the CDRs sequences of the heavy or light chain of the human antibodies of the invention into nanobody or single domain antibody framework sequences, as described for example in PCT/EP93/02214. In one embodiment, the camelid antibody or nanobody binds to at least amino acids residue in domain 2 of HER3 selected from amino acids 265-277, and 315. In one embodiment, the camelid antibody or nanobody binds to at least amino acid residue Lys 268 in domain 2 of HER3.

### Bispecific Molecules and Multivalent Antibodies

In another aspect, the present invention features biparatopic, bispecific or multispecific molecules comprising an antibody or a fragment thereof that binds to an epitope within domain 2 of HER3. The antibody or fragment thereof can be derivatized or linked to another functional molecule, e.g., another peptide or protein (e.g., another antibody or ligand for a receptor) to generate a bispecific molecule that binds to at least two different binding sites or target molecules. The antibody or fragment thereof may in fact be derivatized or linked to more than one other functional molecule to generate biparatopic or multi-specific molecules that bind to more than two different binding sites and/or target molecules; such biparatopic or multi-specific molecules. To create a bispecific molecule, an antibody or fragment thereof can be functionally linked (e.g., by chemical coupling, genetic fusion, non-covalent association or otherwise) to one or more other binding molecules, such as another antibody, antibody fragment, peptide or binding mimetic, such that a bispecific molecule results.

Further clinical benefits may be provided by the binding of two or more antigens within one antibody (Coloma et al., (1997); Merchant et al., (1998); Alt et al., (1999); Zuo et al., (2000); Lu et al., (2004); Lu et al., (2005); Marvin et al., (2005); Marvin et al., (2006); Shen et al., (2007); Wu et al., (2007); Dimasi et al., (2009); Michaelson et al., (2009)). (Morrison et al., (1997) Nature Biotech. 15:159-163; Alt et al. (1999) FEBS Letters 454:90-94; Zuo et al., (2000) Protein Engineering 13:361-367; Lu et al., (2004) JBC 279:2856-2865; Lu et al., (2005) JBC 280:19665-19672; Marvin et al., (2005) Acta Pharmacologica Sinica 26:649-658; Marvin et al., (2006) Curr Opin Drug Disc Develop 9:184-193; Shen et al., (2007) J Immun Methods 218:65-74; Wu et al., (2007) Nat Biotechnol. 11:1290-1297; Dimasi et al., (2009) J Mol Biol. 393:672-692; and Michaelson et al., (2009) mAbs 1:128-141.

The bispecific molecules can be prepared by conjugating the constituent binding specificities, using methods known in the art. For example, each binding specificity of the bispecific molecule can be generated separately and then conjugated to one another, for example, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., (1984) J. Exp. Med. 160:1686; Liu et al., (1985) Proc. Natl. Acad. Sci. USA 82:8648). Other methods include those described in Paulus (1985) Behring Ins. Mitt. No. 78:118-132; Brennan et al., (1985) Science 229:81-83), and Glennie et al., (1987) J. Immunol. 139: 2367-2375). Conjugating agents are SATA and sulfo-SMCC, both available from Pierce Chemical Co. (Rockford, Ill.).

With antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent compounds comprising at least two identical or different fragments of the antibodies binding to HER3. The antibody fragments can be linked together via protein fusion or covalent or non covalent linkage. Tetravalent compounds can be obtained for example by cross-linking antibodies of the antibodies of the invention with an antibody that binds to the constant regions of the antibodies of the invention, for example the Fc or hinge region. Trimerizing domain are described for example in Borean patent EP 1012280B1. Pentamerizing modules are described for example in PCT/EP97/05897.

In one embodiment, a biparatopic/bispecific binds to amino acid residues within domain 2 of HER3.

In another embodiment, the invention pertains to dual function antibodies in which a single monoclonal antibody has been modified such that the antigen binding site binds to more than one antigen, such as a dual function antibody which binds both HER3 and another antigen (e.g., HER1, HER2, and HER4). In another embodiment, the invention pertains to a dual function antibody that targets antigens having the same conformation, for example an antigen that has the same conformation of HER3 in the “closed” or “inactive” state. Examples of antigens with the same conformation of HER3 in the “closed” or “inactive” state include, but are not limited to, HER1 and HER4. Thus, a dual function antibody may bind to both HER3 and HER1; HER3 and HER4, or HER1 and HER4. The dual binding specificity of the dual function antibody may further translate into dual activity, or inhibition of activity. (See e.g., Jenny Bostrom et al., (2009) Science: 323; 1610-1614).

Antibodies with Extended Half Life

The present invention provides for antibodies that specifically bind to an epitope within domain 2 of HER3 which have an extended half-life in vivo.

Many factors may affect a protein's half life in vivo. For examples, kidney filtration, metabolism in the liver, degradation by proteolytic enzymes (proteases), and immunogenic responses (e.g., protein neutralization by antibodies and uptake by macrophages and dentritic cells). A variety of strategies can be used to extend the half life of the antibodies of the present invention. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the invention. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a normative amino acid any place an amber codon is positioned, converting the amber from a stop codon to one that signals incorporation of the chemically specified amino acid.

Recombinant pegylation technology (rPEG) can also be used for serum half-life extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half-life of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology include the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622.

The HER3 antibody or a fragment thereof may also be fused to one or more human serum albumin (HSA) polypeptides, or a portion thereof. HSA, a protein of 585 amino acids in its mature form, is responsible for a significant proportion of the osmotic pressure of serum and also functions as a carrier of endogenous and exogenous ligands. The role of albumin as a carrier molecule and its inert nature are desirable properties for use as a carrier and transporter of polypeptides in vivo. The use of albumin as a component of an albumin fusion protein as a carrier for various proteins has been suggested in WO 93/15199, WO 93/15200, and EP 413 622. The use of N-terminal fragments of HSA for fusions to polypeptides has also been proposed (EP 399 666). Accordingly, by genetically or chemically fusing or conjugating the antibodies or fragments thereof to albumin, can stabilize or extend the shelf-life, and/or to retain the molecule's activity for extended periods of time in solution, in vitro and/or in vivo.

Fusion of albumin to another protein may be achieved by genetic manipulation, such that the DNA coding for HSA, or a fragment thereof, is joined to the DNA coding for the protein. A suitable host is then transformed or transfected with the fused nucleotide sequences, so arranged on a suitable plasmid as to express a fusion polypeptide. The expression may be effected in vitro from, for example, prokaryotic or eukaryotic cells, or in vivo e.g. from a transgenic organism. Additional methods pertaining to HSA fusions can be found, for example, in WO 2001077137 and WO 200306007, incorporated herein by reference. In a specific embodiment, the expression of the fusion protein is performed in mammalian cell lines, for example, CHO cell lines. Altered differential binding of an antibody to a receptor at low or high pHs is also contemplated to be within the scope of the invention. For example, the affinity of an antibody may be modified such that it remains bound to it's receptor at a low pH, e.g., the low pH within a lyzozome, by modifying the antibody to include additional amino acids such as a histine in a CDR of the antibody (See e.g., Tomoyuki Igawa et al. (2010) Nature Biotechnology; 28, 1203-1207).

### Antibody Conjugates

The present invention provides antibodies or fragments thereof that specifically bind to HER3 recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antibody fragment described herein (e.g., a Fab fragment, Fd fragment, Fv fragment, F(ab)₂ fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., (1991) Proc. Natl. Acad. Sci. USA 88:10535-10539; Zheng et al., (1995) J. Immunol. 154:5590-5600; and Vil et al., (1992) Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., (1997) Curr. Opinion Biotechnol. 8:724-33; Harayama, (1998) Trends Biotechnol. 16(2):76-82; Hansson et al., (1999) J. Mol. Biol. 287:265-76; and Lorenzo and Blasco, (1998) Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a HER3 protein may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide (SEQ ID NO: 542) such as the tag provided in a pQE vector (QIAGEN, Inc., 9259 Eton Avenue, Chatsworth, Calif., 91311), among others, many of which are commercially available. As described in Gentz et al., (1989) Proc. Natl. Acad. Sci. USA 86:821-824, for instance, hexa-histidine (SEQ ID NO: 542) provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “flag” tag.

In other embodiments, antibodies of the present invention or fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I) carbon (¹⁴C), sulfur (³⁵S), tritium (³H), indium (¹¹⁵In, ¹¹²In, and ¹¹¹In,), technetium (⁹⁹Tc), thallium (²⁰¹Ti), gallium (⁶⁸Ga, ⁶⁷Ga), palladium (¹⁶³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, 47Sc, ¹⁸⁶Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; and positron emitting metals using various positron emission tomographies, and noradioactive paramagnetic metal ions.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine. In one embodiment, the HER3 antibody, or a fragment thereof is conjugated to a therapeutic moiety, such as a cytotoxin, a drug (e.g., an immunosuppressant) or a radiotoxin. Such conjugates are referred to herein as “immunoconjugates” Immunoconjugates that include one or more cytotoxins are referred to as “immunotoxins.” A cytotoxin or cytotoxic agent includes any agent that is detrimental to (e.g., kills) cells. Examples include taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), ablating agents (e.g., mechlorethamine, thioepa chloraxnbucil, meiphalan, carmustine (BSNU) and lomustine (CCNU), cyclothosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). (See e.g., Seattle Genetics US20090304721).

Other examples of therapeutic cytotoxins that can be conjugated to an antibody or fragment thereof of the invention include duocarmycins, calicheamicins, maytansines and auristatins, and derivatives thereof. An example of a calicheamicin antibody conjugate is commercially available (Mylotarg™; Wyeth-Ayerst).

Cytoxins can be conjugated to antibodies or fragments thereof of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito et al., (2003) Adv. Drug Deliv. Rev. 55:199-215; Trail et al., (2003) Cancer Immunol. Immunother. 52:328-337; Payne, (2003) Cancer Cell 3:207-212; Allen, (2002) Nat. Rev. Cancer 2:750-763; Pastan and Kreitman, (2002) Curr. Opin. Investig. Drugs 3:1089-1091; Senter and Springer, (2001) Adv. Drug Deliv. Rev. 53:247-264.

Antibodies or fragments thereof of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰, and lutetium¹⁷⁷. Method for preparing radioimmunconjugates are established in the art. Examples of radioimmunoconjugates are commercially available, including Zevalin™ (DEC Pharmaceuticals) and Bexxar™ (Corixa Pharmaceuticals), and similar methods can be used to prepare radioimmunoconjugates using the antibodies of the invention. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., (1998) Clin Cancer Res. 4(10):2483-90; Peterson et al., (1999) Bioconjug. Chem. 10(4):553-7; and Zimmerman et al., (1999) Nucl. Med. Biol. 26(8):943-50, each incorporated by reference in their entireties.

Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., (1982) Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Antibody Combinations

An another aspect, the invention pertains to HER3 antibodies, or fragments thereof of the invention used with other therapeutic agents such as another antibodies, small molecule inhibitors, mTOR inhibitors or PI3Kinase inhibitors. Examples include, but are not limited to, the following:

HER1 inhibitors: The HER3 antibodies or fragments thereof can be used with HER1 inhibitors which include, but are not limited to, Matuzumab (EMD72000), Erbitux®/Cetuximab (Imclone), Vectibix®/Panitumumab (Amgen), mAb 806, and Nimotuzumab (TheraCIM), Iressa®/Gefitinib (Astrazeneca); CI-1033 (PD183805) (Pfizer), Lapatinib (GW-572016) (GlaxoSmithKline), Tykerb®/Lapatinib Ditosylate (SmithKlineBeecham), Tarceva®/Erlotinib HCL (OSI-774) (OSI Pharma), and PKI-166 (Novartis), and N-[4-[(3-Chloro-4-fluorophenyl)amino]-7-[[(3″S″)-tetrahydro-3-furanyl]oxy]-6-quinazolinyl]-4(dimethylamino)-2-butenamide, sold under the tradename Tovok® by Boehringer Ingelheim).

HER2 inhibitors: The HER3 antibodies or fragments thereof can be used with HER2 inhibitors which include, but are not limited to, Pertuzumab (sold under the trademark Omnitarg®, by Genentech), Trastuzumab (sold under the trademark Herceptin® by Genentech/Roche), MM-111, neratinib (also known as HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443), lapatinib or lapatinib ditosylate (sold under the trademark Tykerb® by GlaxoSmithKline.

HER3 inhibitors: The HER3 antibodies or fragments thereof can be used with HER3 inhibitors which include, but are not limited to, MM-121, MM-111, IB4C3, 2DID12 (U3 Pharma AG), AMG888 (Amgen), AV-203 (Aveo), MEHD7945A (Genentech), and small molecules that inhibit HER3.

HER4 inhibitors: The HER3 antibodies or fragments thereof can be used with HER4 inhibitors.

PI3K inhibitors: The HER3 antibodies or fragments thereof can be used with PI3 kinase inhibitors which include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036,082 and WO 09/055,730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), BMK120 and BYL719.

mTOR inhibitors: The HER3 antibodies or fragments thereof can be used with mTOR inhibitors which include, but are not limited to, Temsirolimus (sold under the tradename Torisel® by Pfizer), ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as Deforolimus, AP23573 and MK8669 (Ariad Pharm.), and described in PCT Publication No. WO 03/064383), everolimus (RAD001) (sold under the tradename Afinitor® by Novartis), One or more therapeutic agents may be administered either simultaneously or before or after administration of a HER3 antibody or fragment thereof of the present invention.

### Methods of Producing Antibodies of the Invention

### (i) Nucleic Acids Encoding the Antibodies

The invention provides substantially purified nucleic acid molecules which encode polypeptides comprising segments or domains of the HER3 antibody chains described above. Some of the nucleic acids of the invention comprise the nucleotide sequence encoding the HER3 antibody heavy chain variable region, and/or the nucleotide sequence encoding the light chain variable region. In a specific embodiment, the nucleic acid molecules are those identified in Table 1. Some other nucleic acid molecules of the invention comprise nucleotide sequences that are substantially identical (e.g., at least 80%, 90%, 95%, 96%, 97%, 98%, or 99%) to the nucleotide sequences of those identified in Table 1. When expressed from appropriate expression vectors, polypeptides encoded by these polynucleotides are capable of exhibiting HER3 antigen binding capacity.

Also provided in the invention are polynucleotides which encode at least one CDR region and usually all three CDR regions from the heavy or light chain of the antibody or fragment thereof set forth above. Some other polynucleotides encode all or substantially all of the variable region sequence of the heavy chain and/or the light chain of the antibody or fragment thereof set forth above. Because of the degeneracy of the code, a variety of nucleic acid sequences will encode each of the immunoglobulin amino acid sequences.

The nucleic acid molecules of the invention can encode both a variable region and a constant region of the antibody. Some of nucleic acid sequences of the invention comprise nucleotides encoding a mature heavy chain variable region sequence that is substantially identical (e.g., at least 80%, 90%, 95%, 96%, 97%, 98%, or 99%) to the mature heavy chain variable region sequence of a HER3 antibody set forth in Table 1. Some other nucleic acid sequences comprising nucleotide encoding a mature light chain variable region sequence that is substantially identical (e.g., at least 80%, 90%, 95%, 96%, 97%, 98%, or 99%) to the mature light chain variable region sequence of a HER3 antibody set forth in Table 1.

The polynucleotide sequences can be produced by de novo solid-phase DNA synthesis or by PCR mutagenesis of an existing sequence encoding the antibody or fragment thereof. Direct chemical synthesis of nucleic acids can be accomplished by methods known in the art, such as the phosphotriester method of Narang et al., (1979) Meth. Enzymol. 68:90; the phosphodiester method of Brown et al., (1979) Meth. Enzymol. 68:109; the diethylphosphoramidite method of Beaucage et al., (1981) Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H.A. Erlich (Ed.), Freeman Press, NY, N.Y., 1992; PCR Protocols: A Guide to Methods and Applications, Innis et al. (Ed.), Academic Press, San Diego, Calif., 1990; Manila et al., (1991) Nucleic Acids Res. 19:967; and Eckert et al., (1991) PCR Methods and Applications 1:17.

Also provided in the invention are expression vectors and host cells for producing the antibodies or fragments thereof. Various expression vectors can be employed to express the polynucleotides encoding the HER3 antibody chains or fragments thereof. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., (1997) Nat Genet 15:345). For example, nonviral vectors useful for expression of the HER3 polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., (1995) supra; Smith, Annu. Rev. Microbiol. 49:807; and Rosenfeld et al., (1992) Cell 68:143.

The choice of expression vector depends on the intended host cells in which the vector is to be expressed. Typically, the expression vectors contain a promoter and other regulatory sequences (e.g., enhancers) that are operably linked to the polynucleotides encoding an antibody chain or fragment thereof. In some embodiments, an inducible promoter is employed to prevent expression of inserted sequences except under inducing conditions.

Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of antibody chain or fragment thereof. These elements typically include an ATG initiation codon and adjacent ribosome binding site or other sequences. In addition, the efficiency of expression may be enhanced by the inclusion of enhancers appropriate to the cell system in use (see, e.g., Scharf et al., (1994) Results Probl. Cell Differ. 20:125; and Bittner et al., (1987) Meth. Enzymol., 153:516). For example, the SV40 enhancer or CMV enhancer may be used to increase expression in mammalian host cells.

The expression vectors may also provide a secretion signal sequence position to form a fusion protein with polypeptides encoded by inserted antibody or fragment sequences. More often, the inserted antibody or fragment sequences are linked to a signal sequences before inclusion in the vector. Vectors to be used to receive sequences encoding the antibody or fragment light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the antibody or fragment chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies or fragments thereof. Insect cells in combination with baculovirus vectors can also be used.

In some preferred embodiments, mammalian host cells are used to express and produce the antibodies or fragments thereof. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen et al., (1986) Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, (1997) Cell 88:223), agent-enhanced uptake of DNA, and ex vivo transduction. For long-term, high-yield production of recombinant proteins, stable expression will often be desired. For example, cell lines which stably express antibody chains or fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### (ii) Generation of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, (1975) Nature 256:495. Many techniques for producing monoclonal antibody can be employed e.g., viral or oncogenic transformation of B lymphocytes.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5693762 and 6180370 to Queen et al.

In a certain embodiment, the antibodies of the invention are human monoclonal antibodies. Such human monoclonal antibodies directed against HER3 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg et al., (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg et al., (1994) supra; reviewed in Lonberg, (1994) Handbook of Experimental Pharmacology 113:49-101; Lonberg and Huszar, (1995) Intern. Rev. Immunol. 13:65-93, and Harding and Lonberg, (1995) Ann. N.Y. Acad. Sci. 764:536-546). The preparation and use of HuMAb mice, and the genomic modifications carried by such mice, is further described in Taylor et al., (1992) Nucleic Acids Research 20:6287-6295; Chen et al., (1993) International Immunology 5:647-656; Tuaillon et al., (1993) Proc. Natl. Acad. Sci. USA 94:3720-3724; Choi et al., (1993) Nature Genetics 4:117-123; Chen et al., (1993) EMBO J. 12:821-830; Tuaillon et al., (1994) J. Immunol. 152:2912-2920; Taylor et al., (1994) International Immunology 579-591; and Fishwild et al., (1996) Nature Biotechnology 14:845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise HER3 antibodies of the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise HER3 antibodies of the invention. For example, mice carrying both a human heavy chain transchromosome and a human light chain tranchromosome, referred to as “TC mice” can be used; such mice are described in Tomizuka et al., (2000) Proc. Natl. Acad. Sci. USA 97:722-727. Furthermore, cows carrying human heavy and light chain transchromosomes have been described in the art (Kuroiwa et al., (2002) Nature Biotechnology 20:889-894) and can be used to raise HER3 antibodies of the invention.

Human monoclonal antibodies of the invention can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

(iii) Framework or Fc Engineering

Engineered antibodies of the invention include those in which modifications have been made to framework residues within VH and/or VL, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in PCT Publication WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in PCT Publication WO 00/42072 by Presta. Moreover, the binding sites on human IgG1 for FcγR1, FcγRII, FcγRIII and FcRn have been mapped and variants with improved binding have been described (see Shields et al., (2001) J. Biol. Chen. 276:6591-6604).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen”. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., (1999) Nat. Biotech. 17:176-180).

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

### (iv) Methods of Engineering Altered Antibodies

The HER3 antibodies or fragments thereof of the invention having VH and VL sequences or full length heavy and light chain sequences shown herein can be used to create new HER3 antibodies by modifying full length heavy chain and/or light chain sequences, VH and/or VL sequences, or the constant region(s) attached thereto. Thus, in another aspect of the invention, the structural features of a HER3 antibody or fragment thereof are used to create structurally related HER3 antibodies that retain at least one functional property of the antibodies of the invention, such as binding to human HER3 and also inhibiting one or more functional properties of HER3. For example, one or more CDR regions of the antibodies of the present invention, or mutations thereof, can be combined recombinantly with known framework regions and/or other CDRs to create additional, recombinantly-engineered, HER3 antibodies as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing a antibody consisting of: a heavy chain variable region antibody sequence having a CDR1 sequence selected from the group consisting of SEQ ID NOs: 2, 22, 42, 62, 82, 102, 122, 142, 162, 182, 202, 222, 242, 262, 282, 302, 322, 342, 362, 382, 402, 422, 442, 462, 482, 502, and 522; a CDR2 sequence selected from the group consisting of SEQ ID NOs: 3, 23, 43, 63, 83, 103, 123, 143, 163, 183, 203, 223, 243, 263, 283, 303, 323, 343, 363, 383, 403, 423, 443, 463, 483, 503, and 523; and/or a CDR3 sequence selected from the group consisting of SEQ ID NOs: 4, 24, 44, 64, 84, 104, 124, 144, 164, 184, 204, 224, 244, 264, 284, 304, 324, 344, 364, 384, 404, 424, 444, 464, 484, 504, and 524; and a light chain variable region antibody sequence having a CDR1 sequence selected from the group consisting of SEQ ID NOs: 8, 28, 48, 68, 88, 108, 128, 148, 168, 188, 208, 228, 248, 268, 288, 308, 328, 348, 368, 388, 408, 428, 448, 468, 488, 508, and 528; a CDR2 sequence selected from the group consisting of SEQ ID NOs: 9, 29, 49, 69, 89, 109, 129, 149, 169, 189, 209, 229, 249, 269, 289, 309, 329, 349, 369, 389, 409, 429, 449, 469, 489, 509, and 529; and/or a CDR3 sequence selected from the group consisting of SEQ ID NOs: 10, 30, 50, 70, 90, 110, 130, 150, 170, 190, 210, 230, 250, 270, 290, 310, 330, 350, 370, 390, 410, 430, 450, 470, 490, 510, and 530; altering at least one amino acid residue within the heavy chain variable region antibody sequence and/or the light chain variable region antibody sequence to create at least one altered antibody sequence; and expressing the altered antibody sequence as a protein. The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence(s) is one that retains one, some or all of the functional properties of the antibodies or fragments thereof described herein, which functional properties include, but are not limited to, specifically binding to human and/or cynomologus HER3; the antibody binds to HER3 and inhibits HER3 biological activity by inhibiting the HER signaling activity in a phospho-HER assay.

The functional properties of the altered antibodies can be assessed using standard assays available in the art and/or described herein, such as those set forth in the Examples (e.g., ELISAs).

In certain embodiments of the methods of engineering antibodies of the invention, mutations can be introduced randomly or selectively along all or part of an antibody or fragment coding sequence and the resulting modified HER3 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Characterization of the Antibodies of the Invention

The antibodies of the invention can be characterized by various functional assays. For example, they can be characterized by their ability to inhibit biological activity by inhibiting HER signaling in a phospho-HER assay as described herein, their affinity to a HER3 protein (e.g., human and/or cynomologus HER3), the epitope binning, their resistance to proteolysis, and their ability to block HER3 downstream signaling. Various methods can be used to measure HER3-mediated signaling. For example, the HER signaling pathway can be monitored by (i) measurement of phospho-HER3; (ii) measurement of phosphorylation of HER3 or other downstream signaling proteins (e.g. Akt), (iii) ligand blocking assays as described herein, (iv) heterodimer formation, (v) HER3 dependent gene expression signature, (vi) receptor internalization, and (vii) HER3 driven cell phenotypes (e.g. proliferation).

The ability of an antibody to bind to HER3 can be detected by labelling the antibody of interest directly, or the antibody may be unlabelled and binding detected indirectly using various sandwich assay formats known in the art.

In some embodiments, the HER3 antibodies block or compete with binding of a reference HER3 antibody to a HER3. These can be fully human HER3 antibodies described above. They can also be other mouse, chimeric or humanized HER3 antibodies which bind to the same epitope as the reference antibody. The capacity to block or compete with the reference antibody binding indicates that a HER3 antibody under test binds to the same or similar epitope as that defined by the reference antibody, or to an epitope which is sufficiently proximal to the epitope bound by the reference HER3 antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen, such as a HER3 polypeptide or protein. A test antibody competes with the reference antibody for specific binding to the antigen if an excess of the test antibody substantially inhibits binding of the reference antibody. Substantial inhibition means that the test antibody reduces specific binding of the reference antibody usually by at least 10%, 25%, 50%, 75%, or 90%.

There are a number of known competition binding assays that can be used to assess competition of a HER3 antibody with the reference HER3 antibody for binding to a HER3. These include, e.g., solid phase direct or indirect radioimmunoassay (RIA), solid phase direct or indirect enzyme immunoassay (EIA), sandwich competition assay (see Stahli et al., (1983) Methods in Enzymology 9:242-253); solid phase direct biotin-avidin EIA (see Kirkland et al., (1986) J. Immunol. 137:3614-3619); solid phase direct labeled assay, solid phase direct labeled sandwich assay (see Harlow & Lane, supra); solid phase direct label RIA using 1-125 label (see Morel et al., (1988) Molec. Immunol. 25:7-15); solid phase direct biotin-avidin EIA (Cheung et al., (1990) Virology 176:546-552); and direct labeled RIA (Moldenhauer et al., (1990) Scand. J. Immunol. 32:77-82). Typically, such an assay involves the use of purified antigen bound to a solid surface or cells bearing either of these, an unlabelled test HER3-binding antibody and a labelled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if the selected HER3 monoclonal antibodies bind to unique epitopes, each antibody can be biotinylated using commercially available reagents (e.g., reagents from Pierce, Rockford, Ill.). Competition studies using unlabeled monoclonal antibodies and biotinylated monoclonal antibodies can be performed using a HER3 polypeptide coated-ELISA plates. Biotinylated MAb binding can be detected with a strep-avidin-alkaline phosphatase probe. To determine the isotype of a purified HER3-binding antibody, isotype ELISAs can be performed. For example, wells of microtiter plates can be coated with 1 μg/ml of anti-human IgG overnight at 4° C. After blocking with 1% BSA, the plates are reacted with 1 μg/ml or less of the monoclonal HER3 antibody or purified isotype controls, at ambient temperature for one to two hours. The wells can then be reacted with either human IgG1 or human IgM-specific alkaline phosphatase-conjugated probes. Plates are then developed and analyzed so that the isotype of the purified antibody can be determined

To demonstrate binding of monoclonal HER3 antibodies to live cells expressing a HER3 polypeptide, flow cytometry can be used. Briefly, cell lines expressing HER3 (grown under standard growth conditions) can be mixed with various concentrations of a HER3-binding antibody in PBS containing 0.1% BSA and 10% fetal calf serum, and incubated at 4° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

The antibodies or fragments thereof of the invention can be further tested for reactivity with a HER3 polypeptide or antigenic fragment by Western blotting. Briefly, purified HER3 polypeptides or fusion proteins, or cell extracts from cells expressing HER3 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

A number of readouts can be used to assess the efficacy, and specificity, of HER3 antibodies in cell-based assays of ligand-induced heterodimer formation. Activity can be assessed by one or more of the following:

(i) Inhibition of ligand-induced heterodimerisation of HER2 with other EGF family members in a target cell line, for example MCF-7 breast cancer cells. Immunoprecipitation of HER2 complexes from cell lysates can be performed with a receptor-specific antibody, and the absence/presence of other EGF receptors and their biologically relevant ligands within the complex can be analysed following electrophoresis/Western transfer by probing with antibodies to other EGF receptors.

(ii) Inhibition of the activation of signaling pathways by ligand-activated heterodimers. Association with HER3 appears key for other members of the EGF family of receptors to elicit maximal cellular response following ligand binding. In the case of the kinase-defective HER3, HER2 provides a functional tyrosine kinase domain to enable signaling to occur following binding of growth factor ligands. Thus, cells co-expressing HER2 and HER3 can be treated with ligand, for example heregulin, in the absence and presence of inhibitor and the effect on HER3 tyrosine phosphorylation monitored by a number of ways including immunoprecipitation of HER3 from treated cell lysates and subsequent Western blotting using anti-phosphotyrosine antibodies (see Agus op. cit. for details). Alternatively, a high-throughput assay can be developed by trapping HER3 from solubilized lysates onto the wells of a 96-well plate coated with an anti-HER3 receptor antibody, and the level of tyrosine phosphorylation measured using, for example, europium-labelled anti-phosphotyrosine antibodies, as embodied by Waddleton et al., (2002) Anal. Biochem. 309:150-157.

In a broader extension of this approach, effector molecules known to be activated downstream of activated receptor heterodimers, such as mitogen-activated protein kinases (MAPK) and Akt, may be analysed directly, by immunoprecipitation from treated lysates and blotting with antibodies that detect the activated forms of these proteins, or by analysing the ability of these proteins to modify/activate specific substrates.

(iii) Inhibition of ligand-induced cellular proliferation. A variety of cell lines are known to co-express combinations of ErbB receptors, for example many breast and prostate cancer cell lines. Assays may be performed in 24/48/96-well formats with the readout based around DNA synthesis (tritiated thymidine incorporation), increase in cell number (crystal violet staining) etc.

A number of readouts can be used to assess the efficacy, and specificity, of HER3 antibodies in cell-based assays of ligand-independent homo- and heterodimer formation. For example, HER2 overexpression triggers ligand-independent activation of the kinase domain as a result of spontaneous dimer formation. Over expressed HER2 generates either homo- or heterodimers with other HER molecules such as HER1, HER3 and HER4.

Ability of antibodies or fragments thereof to block in vivo growth of tumour xenografts of human tumour cell lines whose tumorigenic phenotype is known to be at least partly dependent on ligand activation of HER3 heterodimer cell signaling e.g. BxPC3 pancreatic cancer cells etc. This can be assessed in immunocompromised mice either alone or in combination with an appropriate cytotoxic agent for the cell line in question. Examples of functional assays are also described in the Example section below.

### Prophylactic and Therapeutic Uses

The present invention provides methods of treating a disease or disorder associated with the HER3 signaling pathway by administering to a subject in need thereof an effective amount of the antibody or fragment thereof of the invention. In a specific embodiment, the present invention provides a method of treating or preventing cancers (e.g., breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis) by administering to a subject in need thereof an effective amount of the antibodies or fragments thereof of the invention. In some embodiments, the present invention provides methods of treating or preventing cancers associated with a HER3 signaling pathway by administering to a subject in need thereof an effective amount of the antibodies of the invention.

In a specific embodiment, the present invention provides methods of treating cancers associated with a HER3 signaling pathway that include, but are not limited to breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis.

The antibodies or fragments thereof of the invention can also be used to treat or prevent other disorders associated with aberrant or defective HER3 signaling, including but are not limited to respiratory diseases, osteoporosis, osteoarthritis, polycystic kidney disease, diabetes, schizophrenia, vascular disease, cardiac disease, non-oncogenic proliferative diseases, fibrosis, and neurodegenerative diseases such as Alzheimer's disease.

Suitable agents for combination treatment with HER3 antibodies include standard of care agents known in the art that are able to modulate the ErbB signaling pathway. Suitable examples of standard of care agents for HER2 include, but are not limited to Herceptin and Tykerb. Suitable examples of standard of care agents for EGFR include, but are not limited to Iressa, Tarceva, Erbitux and Vectibix as described above. Other agents that may be suitable for combination treatment with HER3 antibodies include, but are not limited to those that modulate receptor tyrosine kinases, G-protein coupled receptors, growth/survival signal transduction pathways, nuclear hormone receptors, apoptotic pathways, cell cycle and angiogenesis.

### Diagnostic Uses

In one aspect, the invention encompasses diagnostic assays for determining HER3 and/or nucleic acid expression as well as HER3 protein function, in the context of a biological sample (e.g., blood, serum, cells, tissue) or from individual afflicted with cancer, or is at risk of developing cancer.

Diagnostic assays, such as competitive assays rely on the ability of a labelled analogue (the “tracer”) to compete with the test sample analyte for a limited number of binding sites on a common binding partner. The binding partner generally is insolubilized before or after the competition and then the tracer and analyte bound to the binding partner are separated from the unbound tracer and analyte. This separation is accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte are prepared and compared with the test results in order to quantitatively determine the amount of analyte present in the test sample. These assays are called ELISA systems when enzymes are used as the detectable markers. In an assay of this form, competitive binding between antibodies and HER3 antibodies results in the bound HER3, preferably the HER3 epitopes of the invention, being a measure of antibodies in the serum sample, most particularly, inhibiting antibodies in the serum sample.

A significant advantage of the assay is that measurement is made of inhibiting antibodies directly (i.e., those which interfere with binding of HER3, specifically, epitopes). Such an assay, particularly in the form of an ELISA test has considerable applications in the clinical environment and in routine blood screening.

Another aspect of the invention provides methods for determining HER3 nucleic acid expression or HER3 activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs) on the expression or activity of HER3 in clinical trials.

### Pharmaceutical Compositions

To prepare pharmaceutical or sterile compositions including a antibodies or fragments thereof, the antibodies or fragments thereof are mixed with a pharmaceutically acceptable carrier or excipient. The compositions can additionally contain one or more other therapeutic agents that are suitable for treating or preventing cancer (breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors schwannoma, head and neck cancer, bladder cancer, esophageal cancer, Barretts esophageal cancer, glioblastoma, clear cell sarcoma of soft tissue, malignant mesothelioma, neurofibromatosis, renal cancer, and melanoma, prostate cancer, benign prostatic hyperplasia (BPH), gynacomastica, and endometriosis).

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al., (2003) New Engl. J. Med. 348:601-608; Milgrom et al., (1999) New Engl. J. Med. 341:1966-1973; Slamon et al., (2001) New Engl. J. Med. 344:783-792; Beniaminovitz et al., (2000) New Engl. J. Med. 342:613-619; Ghosh et al., (2003) New Engl. J. Med. 348:24-32; Lipsky et al., (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising antibodies or fragments thereof of the invention can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects. A total weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, or at least 50 mg/kg (see, e.g., Yang et al., (2003) New Engl. J. Med. 349:427-434; Herold et al., (2002) New Engl. J. Med. 346:1692-1698; Liu et al., (1999) J. Neurol. Neurosurg. Psych. 67:451-456; Portielji et al., (2003) Cancer Immunol. Immunother. 52:133-144). The desired dose of antibodies or fragments thereof is about the same as for an antibody or polypeptide, on a moles/kg body weight basis. The desired plasma concentration of the antibodies or fragments thereof is about, on a moles/kg body weight basis. The dose may be at least 15 μg at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more.

For antibodies or fragments thereof of the invention, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the antibodies or fragments thereof of the invention may be calculated using the patient's weight in kilograms (kg) multiplied by the dose to be administered in mg/kg. The dosage of the antibodies or fragments thereof of the invention may be 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the antibodies or fragments thereof of the invention may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the antibodies or fragments thereof of the invention may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the antibodies or fragments thereof of the invention may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 .mu.g/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of antibodies or fragments thereof of the invention may be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side affects (see, e.g., Maynard et al., (1996) A Handbook of SOPs for Good Clinical Practice, Interpharm Press, Boca Raton, Fla.; Dent (2001) Good Laboratory and Good Clinical Practice, Urch Publ., London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., (1983) Biopolymers 22:547-556; Langer et al., (1981) J. Biomed. Mater. Res. 15:167-277; Langer (1982) Chem. Tech. 12:98-105; Epstein et al., (1985) Proc. Natl. Acad. Sci. USA 82:3688-3692; Hwang et al., (1980) Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present invention may also be administered via one or more routes of administration using one or more of a variety of methods known in the art. As will be appreciated by the skilled artisan, the route and/or mode of administration will vary depending upon the desired results. Selected routes of administration for antibodies or fragments thereof of the invention include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically. In one embodiment, the antibodies or fragments thereof of the invention is administered by infusion. In another embodiment, the multispecific epitope binding protein of the invention is administered subcutaneously.

If the antibodies or fragments thereof of the invention are administered in a controlled release or sustained release system, a pump may be used to achieve controlled or sustained release (see Langer, supra; Sefton, (1987) CRC Crit. Ref Biomed. Eng. 14:20; Buchwald et al., (1980), Surgery 88:507; Saudek et al., (1989) N. Engl. J. Med. 321:574). Polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci. Rev. Macromol. Chem. 23:61; see also Levy et al., (1985) Science 228:190; During et al., (1989) Ann. Neurol. 25:351; Howard et al., (1989) J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer, (1990), Science 249:1527-1533). Any technique known to one of skill in the art can be used to produce sustained release formulations comprising one or more antibodies or fragments thereof of the invention. See, e.g., U.S. Pat. No. 4,526,938, PCT publication WO 91/05548, PCT publication WO 96/20698, Ning et al., (1996), Radiotherapy & Oncology 39:179-189, Song et al., (1995) PDA Journal of Pharmaceutical Science & Technology 50:372-397, Cleek et al., (1997) Pro. Int'l. Symp. Control. Rel. Bioact. Mater. 24:853-854, and Lam et al., (1997) Proc. Int'l. Symp. Control Rel. Bioact. Mater. 24:759-760, each of which is incorporated herein by reference in their entirety.

If the antibodies or fragments thereof of the invention are administered topically, they can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising antibodies or fragments thereof are administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the antibodies or fragments thereof of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibodies or fragments thereof of the invention. The two or more therapies may be administered within one same patient visit.

The antibodies or fragments thereof of the invention and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the antibodies or fragments thereof of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The invention provides protocols for the administration of pharmaceutical composition comprising antibodies or fragments thereof of the invention alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof of the invention are administered to a subject in a sequence and within a time interval such that the antibodies of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The invention having been fully described, it is further illustrated by the following examples and claims, which are illustrative and are not meant to be further limiting.

### EXAMPLES

### Example 1

### Methods, Materials and Screening for Antibodies

### (i) Cell Lines

SK-Br-3, BT-474 and MCF-7 cell lines were purchased from ATCC and routinely maintained in growth media supplemented with 10% fetal bovine serum (FBS).

### (ii) Generation of Recombinant Human, Cyno, Mouse and Rat HER3 Vectors

Murine HER3 extracellular domain was PCR amplified from mouse brain cDNA (Clontech) and sequence verified by comparison with Refseq NM_{—}010153. Rat HER3 ECD was reverse transcribed from Rat-2 cell mRNA and sequence verified by comparison with NM_{—}017218. Cynomolgus HER3 cDNA template was generated using RNA from various cyno tissues (Zyagen Laboratories), and the RT-PCR product cloned into pCR®-TOPO-XL (Invitrogen) prior to sequencing of both strands. Human HER3 was derived from a human fetal brain cDNA library (Source) and sequence verified by comparison with NM_{—}001982.

To generate tagged recombinant proteins, human, mouse, rat and cyno HER3 was PCR amplified using Pwo Taq polymerase (Roche Diagnostics). Amplified PCR products were gel purified and cloned into a pDonR201 (Invitrogen) gateway entry vector that had previously been modified to include an in-frame N-terminal CD33 leader sequence and a C-terminal TAG, e.g., FLAG TAG. The TAG allows purification of monomeric proteins via an anti-TAG monoclonal antibody. The target genes were flanked with AttB1 and AttB2 allowing recombination into Gateway adapted proprietary destination vectors (e.g., pcDNA3.1) using the Gateway® cloning technology (Invitrogen). Recombination reactions were performed using a Gateway LR reaction with proprietary destination vectors containing a CMV promoter to create the TAG expression vectors, although any commercially available vector can be used.

Further recombinant HER3 proteins were generated that fused the HER3 ECD upstream of a C-terminal Factor X cleavage site and the human IgG hinge and Fc domain to create an Fc-tagged protein. To achieve this, the various HER3 ECD's were PCR amplified and cloned into a vector (e.g., pcDNA3.1) modified to contain an in-frame C-terminal fusion of Factor X site-Hinge-hFc. The generated open reading frame was flanked with AttB1 and AttB2 sites for further cloning with the Gateway® recombinant cloning technology (Invitrogen). An LR Gateway reaction was used to transfer HER3-Fc into a destination expression construct containing a CMV promoter. HER3 point mutation expression constructs were generated using standard site directed mutagenesis protocols and the resultant vectors sequence verified.

TABLE 2
Generation of HER3 expression vectors.
HER3 amino acid numbering is based on NP_001973 (human),
NP_034283 (mouse) and NP_058914 (rat).

| Name | Description |
|---|---|
| Hu HER3 | CD33-[Human HER3, residues 20-640]-TAG |
| Mu HER3 | CD33-[Murine HER3, residues 20-643]-TAG |
| Rat HER3 | CD33-[Rat HER3, residues 20-643]-TAG |
| Cyno HER3 | CD33-[Cyno HER3, residues 20-643]-TAG |
| HER3 D1-2 | CD33-[Human HER3, residues 20-329]-TAG |
| HER3 D2 | CD33-[Human HER3, residues 185-329]-TAG |
| HER3 D3-4 | CD33-[Human HER3, residues 330-643]-TAG |
| HER3 D4 | CD33-[Human HER3, residues 496-643]-TAG |
| Hu HER3-Fc | [Human HER3, residues 1-643]-Fc |
| Mu HER3-Fc | [Murine HER3, residues 1-643]-Fc |
| Cyno HER3-Fc | [Cyno HER3, residues 1-643]-Fc |
| Rat HER3-Fc | [Rat HER3, residues 1-643]-Fc |
| HER3 D2-Fc | [Human HER3 residues 207-329]-Fc |
| HER3 K267A | CD33-[Human HER3, residues 20-640, K267A]-TAG |
| HER3 L268A | CD33-[Human HER3, residues 20-640, L268A]-TAG |
| HER3 K267A/ | CD33-[Human HER3, residues 20-640, K267A/L268A]- |
| L268A | TAG |

(iii) Expression of Recombinant HER3 Proteins

The desired HER3 recombinant proteins were expressed in HEK293 derived cell lines previously adapted to suspension culture and grown in a Novartis proprietary serum-free medium. Small scale expression verification was undertaken in transient 6-well-plate transfection assays on the basis of lipofection. Large-scale protein production via transient transfection and was performed at the 10-20 L scale in the Wave™ bioreactor system (Wave Biotech). DNA Polyethylenimine (Polysciences) was used as a plasmid carrier at a ratio of 1:3 (w:w). The cell culture supernatants were harvested 7-10 days post transfection and concentrated by cross-flow filtration and diafiltration prior to purification.

### (iv) Tagged Protein Purification

Recombinant tagged HER3 proteins (e.g., TAG-HER3) were purified by collecting the cell culture supernatant and concentrating 10-fold by cross-flow filtration with a 10 kDa cut off filter (Fresenius). An anti-TAG column was prepared by coupling an anti-TAG monoclonal antibody to CNBr activated Sepharose 4B at a final ratio of 10 mg antibody per mL of resin. Concentrated supernatant was applied to a 35 ml anti-Tag column at a flow rate of 1-2 mL/minute. After base-line washing with PBS, bound material was eluted with 100 mM glycine (pH 2.7), neutralized and sterile filtered. Protein concentrations were determined by measuring the absorbance at 280 nm and converting using a theoretical factor of 0.66 AU/mg. The purified protein was finally characterized by SDS-PAGE, N-terminal sequencing and LC-MS.

### (v) Fc Tag Purification

Concentrated cell culture supernatant was applied to a 50 ml Protein A Sepharose Fast Flow column at a flow rate of 1 ml/min. After baseline washing with PBS, the column was washed with 10 column volumes of 10 mM NaH₂PO₄/30% (v/v) Isopropanol, pH 7.3 followed by 5 column volumes of PBS. Finally, bound material was eluted with 50 mM Citrate/140 mM NaCl (pH 2.7), neutralized and sterile filtered.

### (vi) HuCAL PLATINUM® Pannings

For the selection of antibodies recognizing human HER3 multiple panning strategies were employed. Therapeutic antibodies against human HER3 protein were generated by selection of clones having high binding affinities, using as the source of antibody variant proteins a commercially available phage display library, the MorphoSys HuCAL Platinum® library. The phagemid library is based on the HuCAL® concept (Knappik et al., (2000) J Mol Biol 296:57-86) and employs the CysDisplay® technology for displaying the Fab on the phage surface (WO01/05950 to Lohning).

For the isolation of anti-HER3 antibodies, standard as well as RapMAT panning strategies were performed using solid phase, solution, whole cell and differential whole cell panning approaches.

(vii) Solid Phase Panning

To identify anti-HER3 antibodies a variety of solid phase panning strategies were performed using differing recombinant HER3 proteins. To perform each round of solid phase panning, Maxisorp plates (Nunc) were coated with HER3 protein. Tagged proteins were either captured using plates previously coated with anti-Fc (goat or mouse anti-human IgG, Jackson Immuno Research), anti-Tag antibody or via passive adsorption. The coated plates were washed with PBS and blocked. Coated plates were washed twice with PBS prior to the addition of HuCAL Platinum® phage-antibodies for 2 hours at room temperature on a shaker. Bound phages were eluted were added to E. coli TG-land incubated for phage infection. Subsequently infected bacteria were isolated and plated on agar plates. Colonies were scraped off the plates and phages were rescued and amplified. Each HER3 panning strategy comprised of individual rounds of panning and contained unique antigens, antigen concentrations and washing stringency.

(viii) Solution Phase Panning

Each round of solution phase panning was performed using various biotinylated recombinant HER3 proteins in the presence or absence of neuregulin 1-β1 (R&D Systems). Proteins were biotinylated using the EZ-link sulfo-NHS-LC biotinylation kit (Pierce) according to the manufacturers instructions. 800 μl of Streptavidin linked magnetic beads (Dynabeads, Dynal) were washed once with PBS and blocked overnight with Chemiblocker (Chemicon). HuCAL Platinum® phage-antibodies and the appropriate biotinylated HER3 were incubated in a reaction tube. Streptavidin magnetic beads were added for 20 minutes and were collected with a magnetic particle separator (Dynal). Bound phages were eluted from the Dynabeads by adding DTT containing buffer to each tube and added to E. coli TG-1. Phage infection was performed in an identical manner to that described in solid phase panning. Each HER3 panning strategy comprised of individual rounds of panning and contained unique antigens, antigen concentrations and washing stringency. In order to isolate antibodies targeting a specific epitope, competition pannings were performed. In these panning strategies HER3 was incubated and pre-blocked with a reference antibody prior to addition of HuCAL Platinum® phage-antibodies. As an alternative strategy reference antibodies were used to specifically elute phage-antibodies complexed with HER3.

### (ix) Cell Based Panning

For cell pannings, HuCAL Platinum® phage-antibodies were incubated with approximately 10⁷ cells on a rotator for 2 hours at room temperature, followed by centrifugation. The cell pellet was isolated phages were eluted from the cells The supernatant was collected and added to E. coli TG-1 culture continued by the process described above. Two cell based strategies were employed to identify anti-HER3 antibodies:
a) Whole cell panning: In this strategy a variety of intact cell lines were used as the antigens.
b) Differential whole cell panning: In this strategy the antigens sequentially consisted of cells and recombinant HER3 proteins. The cell based pannings were performed as described above whilst solid phase panning protocols were employed when recombinant proteins were utilized as antigens. The washes were conducted using PBS (2-3×) and PBST (2-3×).

### (x) RapMAT™ Library Generation and Pannings

In order to increase antibody binding affinity whilst maintaining library diversity the second round output of both solution and solid phase pannings were entered into the RapMAT™ process whilst the third round output of the whole cell and differential whole cell panning strategies were entered (Prassler et al., (2009) Immunotherapy; 1: 571-583). RapMAT™ libraries were generated by sub-cloning Fab-encoding inserts of phages selected via panning into the display vector pMORPH° 25_bla_LHC and were further digested to either generate H-CDR2 RapMAT™ libraries and L-CDR3 RapMAT™ libraries by using specific restriction enzymes. The inserts were replaced with TRIM maturation cassettes (Virnekas et al., (1994) Nucleic Acids Research 22:5600-5607) for H-CDR2 or L-CDR3 according to pool composition. Library sizes were estimated to range between 8×10⁶-1×10⁸ clones. RapMAT antibody-phage were produced and subjected to two further rounds of solution, solid phase or cell based panning using the experimental methods described previously.

This extensive panning strategy, involving an iterative refinement of library design was specifically developed to bias screening away from pure ligand-competitive antibodies by including ligand-blocking antibodies directly in the pannings. Secondly, the FAB to IgG conversion process was adapted to maximize the recovery of candidate clones and ensure that all selective binders were profiled in functional assays. From 44 initial pannings, yielding over x clones, only three antibody families with the desired property of blocking both ligand-dependent and independent signal transduction. Family A that binds isolated domains 1-2 and 2 of Her3. Family B that binds isolated domains 3-4, but not 4 alone; and family C, which binds domain 3.

### Example 2

### Transient Expression of Anti-HER3 IgG's

Suspension adapted HEK293-6E cells were cultivated in a BioWave20. The cells were transiently transfected with the relevant sterile DNA: PEI-MIX and further cultivated. After transfection, cells were removed by crossflow filtration using Fresenius filters. The cell free material was concentrated with crossflow filtration using a cut off filter (Fresenius) and the concentrate was sterile filtered through a stericup filter. The sterile supernatant was stored at 4° C.

### Example 3

### Purification of Anti-HER3 IgG

The purification of IgG was performed on a ÄKTA 100 explorer Air chromatography system in a cooling cabinet, using a XK16/20 column with 25 mL of self-packed MabSelect SuRe resin (all GE Healthcare). All flow rates were 3 5 mL/min, except for loading, at a pressure limit of 5 bar. The column was equilibrated with 3 column volumes of PBS prior to loading the filtered fermentation supernatant. The column was washed with PBS. IgG was eluted with a pH gradient, starting at citrate/NaCl (pH 4.5), going linearly down to citrate/NaCl (pH 2.5), followed by a constant step of the same pH 2.5 buffer. The IgG containing fractions were pooled and immediately neutralized and sterile filtered (Millipore Steriflip, 0.22 um). OD₂₈₀ was measured and the protein concentration calculated based on the sequence data. The pools were separately tested for aggregation (SEC-MALS) and purity (SDS-PAGE and MS).

### Example 4

### Expression and Purification of HuCAL®-Fab Antibodies in E. Coli

Expression of Fab fragments encoded by pMORPH®X9_Fab_MH in TG-1 cells was carried out in shaker flask cultures using YT medium supplemented with chloramphenicol. Cultures were shaken until the OD600 nm reached 0.5. Expression was induced by addition of IPTG (isopropyl-β-D-thiogalactopyranoside). Cells were disrupted using lysozyme. His₆-tagged Fab fragments (‘His₆’ disclosed as SEQ ID NO: 542) were isolated via IMAC (Bio-Rad). Buffer exchange to 1× Dulbecco's PBS (pH 7.2) was performed using PD10 columns. Samples were sterile filtered. Protein concentrations were determined by UV-spectrophotometry. The purity of the samples was analyzed in denaturing, reducing 15% SDS-PAGE. The homogeneity of Fab preparations was determined in native state by size exclusion chromatography (HP-SEC) with calibration standards.

### Example 5

### HER3 Antibody Affinity (K_{D}) Measurements by Solution Equilibrium Titration (SET)

Affinity determination in solution was essentially performed as previously described (Friguet et al., (1985) J Immunol Methods 77:305-19). In order to improve the sensitivity and accuracy of the SET method, it was transferred from classical ELISA to ECL based technology (Haenel et al., (2005) Anal Biochem 339:182-84).

Unlabeled HER3-Tag (human, rat, mouse or cyno) described previously was used for affinity determination by SET.

The data was evaluated with XLfit software (ID Business Solutions) applying customized fitting models. For K_{D} determination of each IgG the following model was used (modified according to Piehler, et al (Piehler et al., (1997) J Immunol Methods 201:189-206).

$$y = \frac{2 {B}_{max}}{[ IgG ]} ( \frac{[ IgG ]}{2} - \frac{{( \frac{x + [ IgG ] + {K}_{D}}{2} - \sqrt{\frac{{( x + [ IgG ] + {K}_{D} )}^{2}}{2} - x [ IgG ]} )}^{2}}{2 [ IgG ]} )$$

[IgG]: applied total IgG concentration
x: applied total soluble antigen concentration (binding sites)
Bₘₐₓ: maximal signal of IgG without antigen
K_{D}: affinity

### Example 6

### Antibody Cell Binding Determination by FACS

The binding of antibodies to endogenous human antigen expressed on human cancer cells was accessed by FACS. In order to determine antibody EC₅₀ values SK-Br-3 cells were harvested with accutase and diluted to 1×10⁶ cells/mL in FACS buffer (PBS/3% FBS/0.2% NaN₃). 1×10⁵ cells/well were added to each well of a 96-well plate (Nunc) and centrifuged at 210 g for 5 minutes at 4° C. before removing the supernatant. Serial dilutions of test antibodies (diluted in 1:4 dilution steps with FACS buffer) were added to the pelleted cells and incubated for 1 hour on ice. The cells were washed and pelleted three times with 100 μL FACS buffer. PE conjugated goat anti-human IgG (Jackson ImmunoResearch) diluted 1/200 with FACS buffer were added to the cells and incubated on ice for 1 hour. Additional washing steps were performed three times with 100 μL FACS buffer followed by centrifugation steps at 210 g for 5 minutes at 4° C. Finally, cells were resuspended in 200 μL FACS buffer and fluorescence values were measured with a FACSArray (BD Biosciences). The amount of cell surface bound anti-HER3 antibody was assessed by measuring the mean channel fluorescence.

### Example 7

### HER3 Domain and Mutant Binding

96-well Maxisorp plates (Nunc) were coated overnight with 200 ng of the appropriate recombinant human protein (HER3-Tag, D1-2-Tag, D2-Tag, D3-4-Tag, D4-Tag, HER3 K267A-Tag, HER3 L268A-Tag, HER3 K267A/L268A and a tagged irrelevant control). All wells were then washed with PBS/0.1% Tween-20, blocked with PBS/1% BSA/0.1% Tween-20 and washed with PBS/0.1% Tween-20. Anti-HER3 antibodies were added to the relevant wells up to a final concentration of 10 μg/mL and incubated at room temperature. Plates were washed with PBS/0.1% Tween-20 prior to the addition of the appropriate peroxidase linked detection antibody diluted 1/10000 in PBS/1% BSA/0.1% Tween-20. The detection antibodies used were goat anti-mouse (Pierce, 31432), rabbit anti-goat (Pierce, 31402) and goat anti-human (Pierce, 31412). Plates were incubated at room temperature before washing with PBS/0.1% Tween-20. 100 μl TMB (3,3′,5,5′ tetramethyl benzidine) substrate solution (BioFx) was added to all wells before stopping the reaction with 50 μl 2.5% H₂SO₄. The extent of HER3 antibody binding to each recombinant protein was determined by measuring the OD₄₅₀ using a SpectraMax plate reader (Molecular Devices). Where appropriate, dose response curves were analyzed using Graphpad Prism.

### Example 8

### Antibody Cross-Competition by ELISA

Antibody A was coated at a constant amount on Maxisorp plates and tested for competition of binding to HER3 with increasing amounts of antibody B in solution. Maxisorp plates were coated with 24 ng/well antibody A in PBS, incubated overnight at 4° C. and then washed with PBST. Plates were blocked with 3% BSA/PBS for 1 hour at room temperature. Antibody B was titrated in 1:3 steps and incubated in molar excess with biotinylated HER3-Tag for 1 hour at room temperature in solution. HER3/antibody B complexes were then added to the antibody A coated plate for 30 minutes and bound complexes detected by quantifying the amount of biotinylated HER3-Tag. Blocked plates were subsequently washed with PBST, preformed HER3/antibody B complexes added and incubated for 30 minutes at room temperature with gentle shaking. Plates were subsequently washed with excess PBST and incubated for 1 hour with Streptavidin-AP diluted 1:5000 in 1% BSA/0.05% Tween20/PBS. Plates were washed with PBST, AttoPhos solution (1:5 diluted in H₂O) was added and fluorescent signals were measured at 535 nm following excitation at 430 nm.

If antibody A did not compete with antibody B for binding to HER3 then a high level of HER3 was detected. In contrast, for competitive antibodies or antibodies with partially overlapping epitopes, HER3 signals were significantly decreased when compared to IgG controls.

### Example 9

### Phospho-HER3 In Vitro Cell Assays

MCF-7 cells were routinely maintained in DMEM/F12, 15 mM HEPES, L-glutamine, 10% FBS, BT474 in DMEM, 10% FBS and SK-Br-3 in McCoy's 5a, 10% FBS, 1.5 mM L-glutamine. Sub-confluent cells were trypsinized, washed with PBS and diluted to 5×10⁵ cells/mL. 100 μL of cell suspension was then added to each well of a 96-well flat bottomed plate (Nunc) to give a final density of 5×10⁴ cells/well. MCF7 cells were allowed to attach for approximately 3 hours before the media was exchanged for starvation media containing 0.5% FBS. All plates were then incubated overnight at 37° C. prior to treatment with the appropriate concentration of HER3 antibodies for 80 minutes at 37° C. MCF7 cells were treated with 50 ng/mL NRG1 for the final 20 minutes to stimulate HER3 and AKT phosphorylation whilst BT474/SK-Br-3 cells required no additional stimulation. All media was gently aspirated and the cells washed with ice-cold PBS containing 1 mM CaCl₂ and 0.5 mM MgCl₂ (Gibco). The cells were lysed by adding 50 μL ice-cold lysis buffer (20 mM Tris (pH8.0)/137 mM NaCl/10% Glycerol/2 mM EDTA/1% NP-40/1 mM sodium orthovanadate/1× Phospho-Stop/1× Complete mini protease inhibitors (Roche)/0.1 mM PMSF) and incubated on ice with shaking for 30 minutes. Lysates were then collected and spun at 1800 g for 15 minutes at 4° C. to remove cell debris.

HER3 capture plates were generated using a carbon plate (Mesoscale Discovery) coated overnight at 4° C. with 20 μL of 4 μg/mL MAB3481 capture antibody (R&D Systems) diluted in PBS and subsequently blocked with 3% bovine serum albumin in 1× Tris buffer (Mesoscale Discovery)/0.1% Tween-20. HER3 was captured by adding the appropriate amount of lysate and incubating the plate at room temperature for one hour with shaking before the lysate was aspirated and the wells washed with 1× Tris buffer (Mesoscale Discovery)/0.1% Tween-20. Phosphorylated HER3 was detected using 1:8000 anti-pY1197 antibody (Cell Signaling) prepared in 3% milk/1× Tris/0.1% Tween-20 by incubating with shaking at room temperature for 1 hour. The wells were washed four times with 1× Tris/0.1% Tween-20 and phosphorylated proteins were detected by incubating with S-Tag labelled goat anti-rabbit Ab (#R32AB) diluted in 3% blocking buffer for one hour at room temperature. Each well was aspirated and washed four times with 1× Tris/0.1% Tween-20 before adding 20 μL of Read buffer T with surfactant (Mesoscale Discovery) and the signal quantified using a Mesoscale Sector Imager.

### Example 10

### Phospho-Akt (S473) In Vitro Cell Assays

Sub-confluent MCF7, SK-Br-3 and BT-474 cells were grown in complete media were harvested with accutase (PAA Laboratories) and resuspended in the appropriate growth media at a final concentration of 5×10⁵ cells/mL. 100 μL of cell suspension was then added to each well of a 96-well flat bottomed plate (Nunc) to yield a final density of 5×10⁴ cells/well. MCF7 cells were allowed to attach for approximately 3 hours before the media was exchanged for starvation media containing 0.5% FBS. All plates were then incubated overnight at 37° C. prior to treatment with the appropriate concentration of HER3 antibodies for 80 minutes at 37° C. MCF7 cells were treated with 50 ng/mL NRG1 for the final 20 minutes to stimulate HER3 and AKT phosphorylation whilst SK-Br-3 cells required no additional stimulation. All media was gently aspirated and the cells washed with ice-cold PBS containing 1 mM CaCl₂ and 0.5 mM MgCl₂ (Gibco). The cells were lysed by adding 50 μL ice-cold lysis buffer (20 mM Tris (pH8.0)/137 mM NaCl/10% Glycerol/2 mM EDTA/1% NP-40/1 mM sodium orthovanadate/Aprotinin (10 μg/mL)/Leupeptin (10 μg/mL)) and incubated on ice with shaking for 30 minutes. Lysates were then collected and spun at 1800 g for 15 minutes at 4° C. to remove cell debris. 20 μL of lysate was added to a multi-spot 384-well Phospho-Akt carbon plate (Mesoscale Discovery) that had previously been blocked with 3% BSA/1× Tris/0.1% Tween-20. The plate was incubated at room temperature for two hours with shaking before the lysate was aspirated and the wells washed four times with 1× Tris buffer (Mesoscale Discovery)/0.1% Tween-20. Phosphorylated Akt was detected using 20 μL of SULFO-TAG anti-phospho-Akt (S473) antibody (Mesoscale Discovery) diluted 50-fold in 1% BSA/1× Tris/0.1% Tween-20 by incubating with shaking at room temperature for 2 hours. The wells were washed four times with 1× Tris/0.1% Tween-20 before adding 20 μL of Read buffer T with surfactant (Mesoscale Discovery) and the signal quantified using a Mesoscale Sector Imager.

### Example 11

### Cell-Line Proliferation Assays

SK-Br-3 cells were routinely cultured in McCoy's 5A medium modified, supplemented with 10% fetal bovine serum and BT-474 cells were cultured in DMEM supplemented with 10% FBS. Sub-confluent cells were trypsinized, washed with PBS, diluted to 5×10⁴ cells/mL with growth media and plated in 96-well clear bottom black plates (Costar 3904) at a density of 5000 cells/well. The cells were incubated overnight at 37° C. before adding the appropriate concentration of HER3 antibody (typical final concentrations of 10 or 1 μg/mL). The plates were returned to the incubator for 6 days before assessing cell viability using CellTiter-Glo (Promega). 100 μL of CellTiter-Glo solution was added to each well and incubated at room temperature with gentle shaking for 10 minutes. The amount of luminescence was determined using a SpectraMax plate reader (Molecular Devices). The extent of growth inhibition obtained with each antibody was calculated by comparing the luminscence values obtained with each HER3 antibody to a standard isotype control antibody.

For proliferation assays MCF-7 cells were routinely cultured in DMEM/F12 (1:1) containing 4 mM L-Glutamine/15 mM HEPES/10% FBS. Sub-confluent cells were trypsinized, washed with PBS and diluted to 1×10⁵ cells/mL with DMEM/F12 (1:1) containing 4 mM L-Glutamine/15 mM HEPES/10 μg/mL Human Transferrin/0.2% BSA. Cells were plated in 96-well clear bottom black plates (Costar) at a density of 5000 cells/well. The appropriate concentration of HER3 antibody (typical final concentrations of 10 or 1 μg/mL) was then added. 10 ng/mL of NRG1-β1 EGF domain (R&D Systems) was also added to the appropriate wells to stimulate cell growth. The plates were returned to the incubator for 6 days before assessing cell viability using CellTiter-Glo (Promega). The extent of growth inhibition obtained with each antibody was calculated by subtracting the background (no neuregulin) luminscence values and comparing the resulting values obtained with each anti-HER3 antibody to a standard isotype control antibody.

### Example 12

### In Vivo BxPC3 Efficacy Studies

BxPC3 cells were cultured in RPMI-1640 medium containing 10% heat-inactivated fetal bovine serum without antibiotics until the time of implantation.

Female athymic nu/nu Balb/C mice (Harlan Laboratories) were implanted subcutaneously with 10×106 cells in a mixture of 50% phosphate buffered saline with 50% matrigel. The total injection volume containing cells in suspension was 200 μL. Once tumors had reached approximately 200 mm3 in size, animals were enrolled in the efficacy study. In general, a total of 10 animals per group were enrolled in studies. Animals were excluded from enrollment if they exhibited unusual tumor growth characteristics prior to enrollment.

Animals were dosed intravenously via lateral tail vein injection. Animals were on a 20 mg/kg, twice weekly schedule for the duration of the study. Tumor volume and T/C values were calculated as detailed for the BT-474 studies.

### Example 13

### In Vivo BT-474 Efficacy Studies

BT-474 cells were cultured in DMEM containing 10% heat-inactivated fetal bovine serum without antibiotics until the time of implantation.

One day before cell inoculation, female athymic nu/nu Balb/C mice (Harlan Laboratories) were implanted subcutaneously with a sustained release 17β-estradiol pellet (Innovative Research of America) to maintain serum estrogen levels. One day after 17β-estradiol pellet implantation, 5×106 cells were injected orthotopically into the 4th mammary fatpad in a suspension containing 50% phenol red-free matrigel (BD Biosciences) in Hank's balanced salt solution. The total injection volume containing cells in suspension was 200 μL. 20 days following cell implantation animals with a tumor volume of approximately 200 mm³ were enrolled in the efficacy study. In general, a total of 10 animals per group were enrolled in efficacy studies.

For single-agent studies, animals were dosed intravenously via lateral tail vein injection with control IgG or MOR13759. Animals were on a 20 mg/kg, twice weekly dosing schedule for the duration of the study. For the duration of the studies, tumor volume was measured by calipering twice per week. Percent treatment/control (T/C) values were calculated using the following formula:

% T/C=100×ΔT/ΔC if ΔT>0

where:

T=mean tumor volume of the drug-treated group on the final day of the study;
ΔT=mean tumor volume of the drug-treated group on the final day of the study−mean tumor volume of the drug-treated group on initial day of dosing;
C=mean tumor volume of the control group on the final day of the study; and
ΔC=mean tumor volume of the control group on the final day of the study−mean tumor volume of the control group on initial day of dosing.

Body weight was measured twice per week and dose was body weight adjusted. The % change in body weight was calculated as (BWcurrent−BWinitial)/(BWinitial)×100. Data is presented as percent body weight change from the day of treatment initiation.

All data were expressed as mean±standard error of the mean (SEM). Delta tumor volume and body weight were used for statistical analysis. Between groups comparisons were carried out using a one-way ANOVA followed by a post hoc Tukey. For all statistical evaluations the level of significance was set at p<0.05. Significance compared to the vehicle control group is reported.

### Results and Discussion

Collectively, these results show that a class of antibodies bind to amino acid residues within domain 2. Binding of these antibodies inhibits both ligand-dependent and ligand-independent signaling.

### (i) Affinity Determination

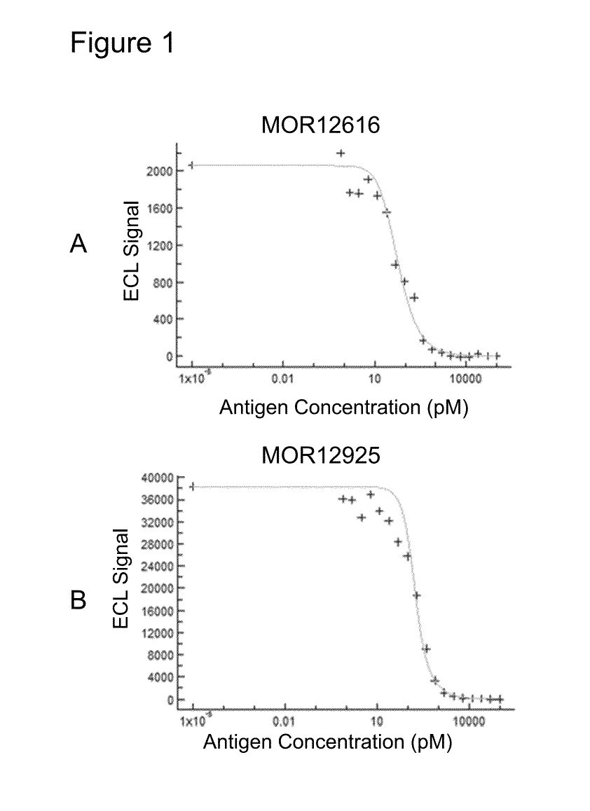

Antibody affinity was determined by solution equilibrium titration (SET) as described above. The results are summarized in Table 3 and example titration curves for MOR12616 and MOR12925 are contained in FIG. 1. The data indicate that a number of antibodies were identified that tightly bound human, cyno, rat and murine HER3.

TABLE 3
K_{D} values of anti-HER3 IgGs as determined by solution equilibrium
titration (SET).

| | SET K_{D} (pM) | | | |
|---|---|---|---|---|
| | hu | | mu | |
| MOR# | HER3-Tag | cy HER3-Tag | HER3-Tag | ra HER3-Tag |
| MOR12509 | 78 | 320 | 250 | 480 |
| MOR12510 | 55 | 400 | 200 | 750 |
| MOR12616 | 20 | 74 | 41 | 122 |
| MOR12923 | nd | nd | nd | nd |
| MOR12924 | nd | nd | nd | nd |
| MOR12925 | 28 | 74 | 38 | 100 |
| MOR13750 | 160 | nd | nd | nd |
| MOR13752 | 96 | nd | nd | nd |
| MOR13754 | 290 | nd | nd | nd |
| MOR13755 | 24 | nd | nd | nd |
| MOR13756 | 25 | nd | nd | nd |
| MOR13758 | 21 | nd | nd | nd |
| MOR13759 | 51 | nd | nd | nd |
| MOR13761 | 28 | nd | nd | nd |
| MOR13762 | 25 | nd | nd | nd |
| MOR13763 | 27 | nd | nd | nd |
| MOR13765 | 64 | nd | nd | nd |
| MOR13766 | 54 | nd | nd | nd |
| MOR13767 | 24 | nd | nd | nd |
| MOR13768 | 37 | nd | nd | nd |
| MOR13867 | 30 | nd | nd | nd |
| MOR13868 | 133 | nd | nd | nd |
| MOR13869 | 230 | nd | nd | nd |
| MOR13870 | 173 | nd | nd | nd |
| MOR13871 | 193 | nd | nd | nd |
| MOR14535 | 235 | 215 | 125 | 315 |
| MOR14536 | 72 | 44 | 29 | 64 |

Hu (human),
Cy (cynomolgus),
Mu (murine) and
ra (rat)

### (ii) SK-Br-3 Cell EC₅₀ Determination

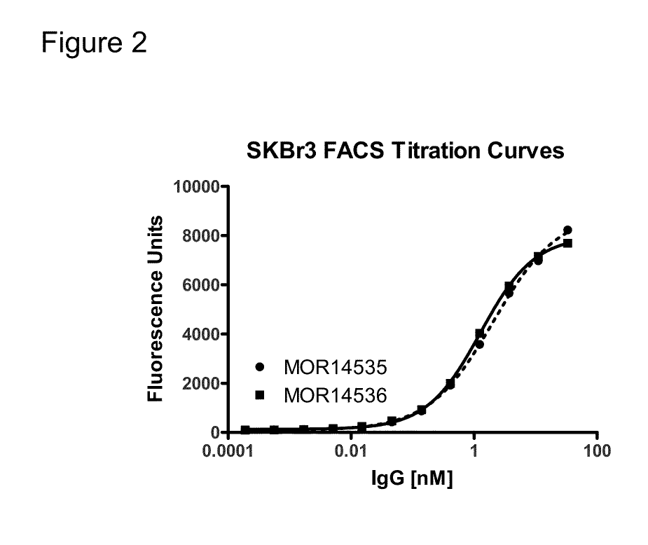

The ability of the identified antibodies to bind HER3 expressing cells was determined by calculating EC₅₀ values for their binding to the HER2 amplified cell line SK-Br-3 (see FIG. 2 and Table 4).

TABLE 4
FACS EC₅₀ values of anti-HER3 IgG on cells.

| | FACS EC₅₀ (pM) | | |
|---|---|---|---|
| MOR# | SK-Br-3 | B16-F10 | JTC12 |
| 14535 | 2075 | 524 | 65110 |
| 14536 | 1272 | 625 | 1362 |

(iii) HER3 Domain Binding

A subset of anti-HER3 antibodies were characterized for their ability to bind the various extracellular domains of human HER3 in an ELISA assay. To achieve this, the extracellular domain of HER3 was divided into its four constitutive domains and various combinations of these domains were cloned, expressed and purified as independent proteins as described above. Using this strategy the following domains were successfully generated as soluble proteins: domains 1 and 2 (D1-2), domain 2 (D2), domains 3 and 4 (D3-4) and domain 4 (D4). The integrity of each isolated domain was previously confirmed using a panel of internally generated antibodies as positive controls.

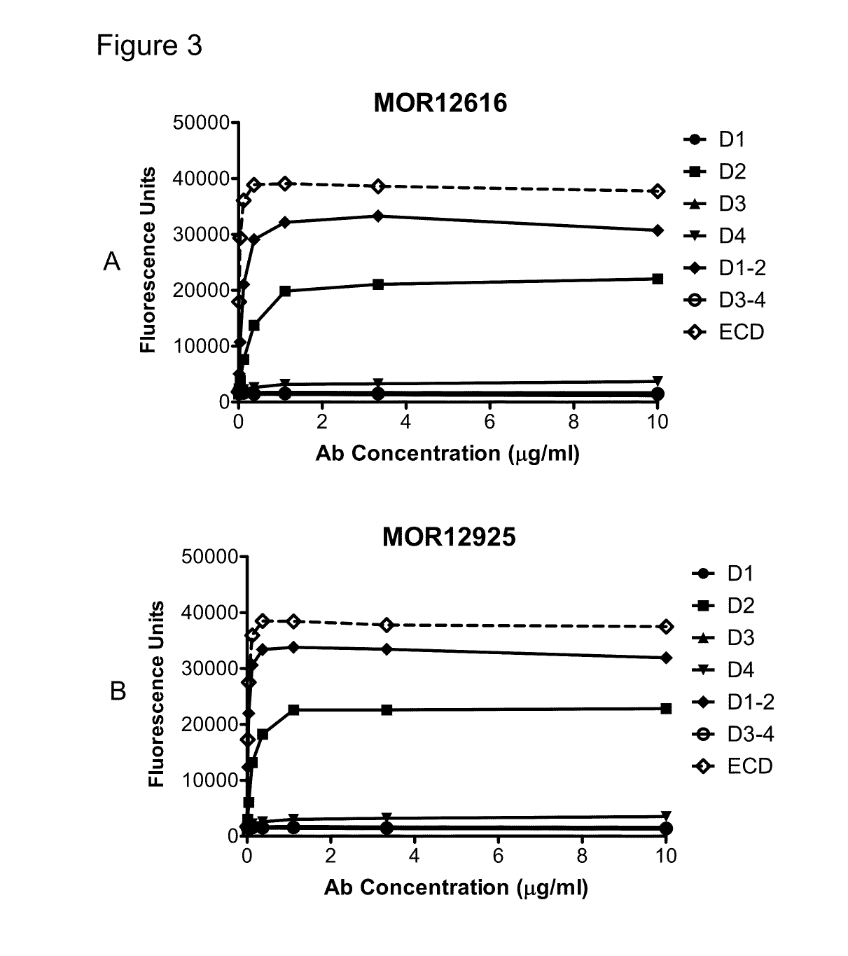

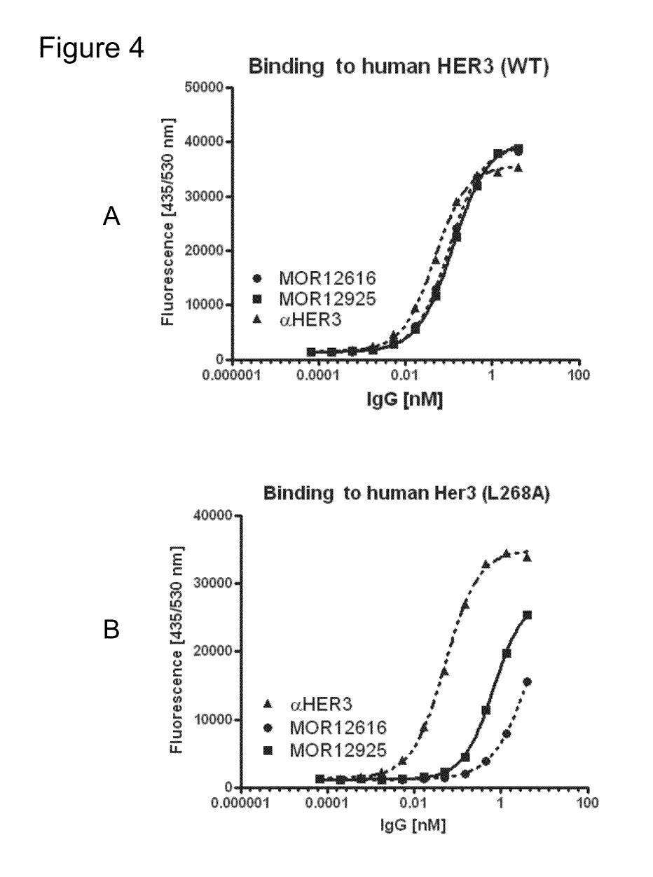

As shown in FIG. 3 MOR12616 and MOR12925 were both observed to bind the HER3 extracellular domain, isolated D1-2 and isolated D2 protein. No binding was observed with D3-4 or D4 proteins. This binding data suggests that this family of antibodies recognize an epitope primarily contained within domain 2. To further confirm the epitope we determined the impact of mutating residues within D2 upon antibody binding. As determined by both binding ELISA (FIG. 4) and SET (Table 5), mutation of Lysine²⁶⁸ to alanine severely disrupted antibody binding thus confirming that binding epitope is contained with domain 2.

TABLE 5
K_{D} values of anti-HER3 IgG binding to mutant forms of HER3
as determined by solution equilibrium titration (SET).

| | SET K_{D} (pM) | | | |
|---|---|---|---|---|
| MOR# | HER3 ECD | K267A | L268A | K267A/L268A |
| 13768 | 37 | 99 | No binding | No binding |

### v) Epitope Competition ELISA's

To further refine the epitope of this class of anti-HER3 antibodies we performed epitope competition studies on a sub-set of antibodies versus a number of proprietary anti-HER3 antibodies whose epitopes had previously been characterized. Epitope competition experiments consist of Antibody A (e.g. MOR12925 or MOR12616) being immobilized on a plate and testing its ability to capture HER3/antibody B complexes from solution. If antibody A does not compete with antibody B for binding to HER3 then HER3 complexes are captured from solution. In contrast, if antibody A possesses an identical or overlapping epitope to antibody B then HER3 complexes cannot be captured. Using this method, allosteric competitors may also be identified. In this instance binding of antibody B to HER3 could induce a conformational change that masks the antibody A epitope. Thus antibody A and antibody B may appear to directly compete even though their HER3 binding residues may be distal from each other.

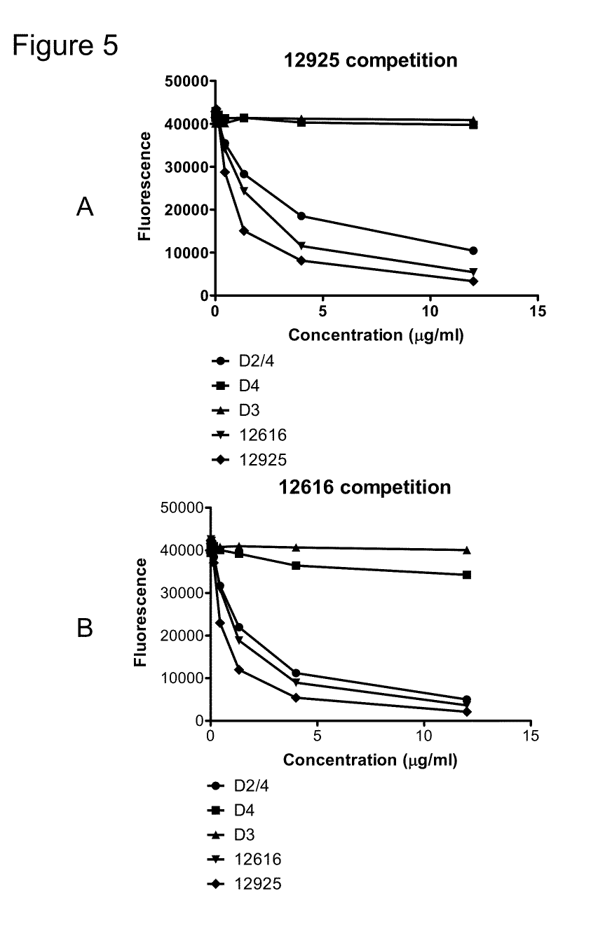

Example epitope competition data for MOR12925 and MOR12616 are illustrated in FIG. 5. As can be seen from the data both MOR12925 and MOR12616 effectively cross-compete for binding to HER3 thus demonstrating that these highly related antibodies probably bind the same HER3 epitope. Cross-competition was also observed with an antibody (D2/4) whose epitope has previously been mapped to residues contained within domains 2 and 4. Interestingly, no competition was observed with an antibody (D4) that binds isolated HER3 domain 4. This data suggests that both MOR12925 and MOR12616 bind an epitope contained within domain 2, which is consistent with our previous domain binding ELISA. Since antibody D2/4 has been demonstrated to interact with amino acid residues 265-277, 315 within domain 2 of HER3 it can be inferred that some of these residues may also be critical for MOR12925 and MOR12616 binding.

### (vi) Inhibition of Cell Signaling

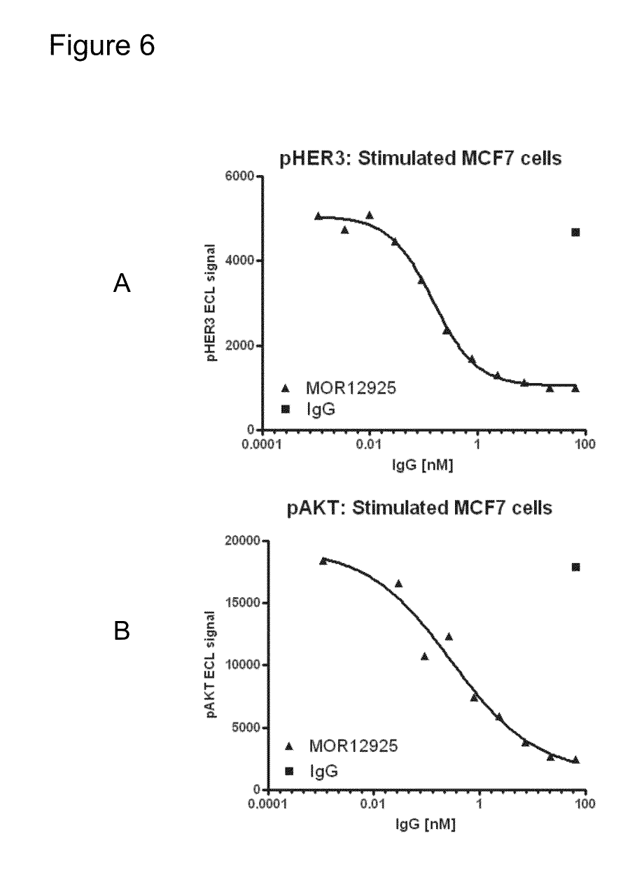

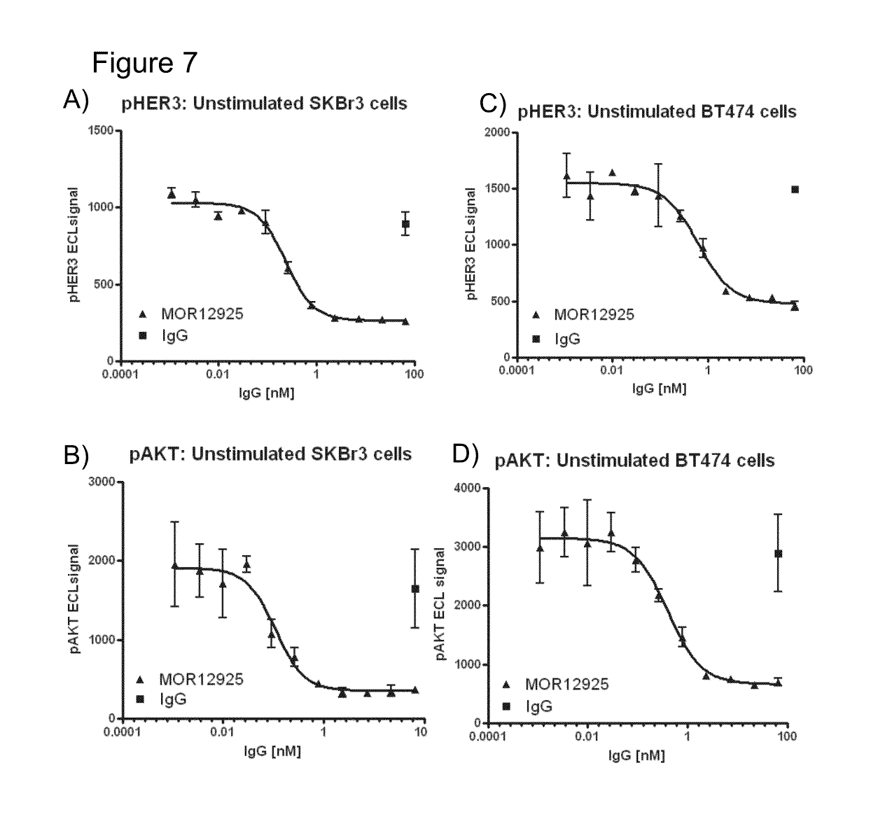

To ascertain the effect of anti-HER3 antibodies upon ligand dependent HER3 activity MCF7 cells were incubated with IgG prior to stimulation with neuregulin. Example inhibition curves are illustrated in FIG. 6 and summarized in Table 6. The effect of anti-HER3 antibodies upon HER2-mediated HER3 activation was also studied using the HER2 amplified cell lines SK-Br-3 and BT474 (FIG. 7 and Table 6).

TABLE 6
pHER3 IC₅₀ and extent of inhibition values of anti-HER3 IgG in MCF7,
BT474 and SK-Br-3 cells.

| | MCF7 | | SK-Br-3 | | | |
|---|---|---|---|---|---|---|
| | pHER3 | | pHER3 | | BT474 pHER3 | |
| | IC₅₀ | % | IC₅₀ | % | IC₅₀ | % |
| MOR# | (pM) | inhibition | (pM) | inhibition | (pM) | inhibition |
| MOR12509 | 712 | 70 | 483 | 76 | | 84 |
| MOR12510 | 450 | 73 | 157 | 78 | 34 | 85 |
| MOR12616 | 245 | 76 | 142 | 75 | 436 | 58 |
| MOR12923 | 540 | 83 | 70 | 74 | 1047 | 61 |
| MOR12924 | 631 | 80 | 123 | 76 | 1586 | 62 |
| MOR12925 | 327 | 81 | 136 | 75 | 2161 | 63 |
| MOR13750 | nd | nd | 1437 | 60 | 6676 | 44 |
| MOR13752 | nd | nd | 1000 | 71 | 4244 | 45 |
| MOR13754 | nd | nd | 606 | 63 | 3898 | 49 |
| MOR13755 | 331 | 79 | 217 | 80 | 657 | 73 |
| MOR13756 | nd | nd | 95 | 78 | 322 | 66 |
| MOR13758 | nd | nd | 91 | 77 | 382 | 67 |
| MOR13759 | 277 | 74 | 124 | 77 | 431 | 70 |
| MOR13761 | nd | nd | 62 | 76 | 364 | 56 |
| MOR13762 | nd | nd | 90 | 70 | 271 | 41 |
| MOR13763 | nd | nd | 107 | 77 | 19520 | 60 |
| MOR13765 | 374 | 71 | 227 | 79 | 806 | 61 |
| MOR13766 | 354 | 72 | 106 | 79 | 708 | 62 |
| MOR13767 | nd | nd | 130 | 78 | 496 | 61 |
| MOR13768 | 412 | 75 | 139 | 81 | 437 | 70 |
| MOR13867 | 267 | 78 | 107 | 83 | 643 | 71 |
| MOR13868 | 189 | 75 | 205 | 83 | 894 | 68 |
| MOR13869 | 427 | 64 | 263 | 77 | 1327 | 55 |
| MOR13870 | 84 | 71 | 110 | 81 | 535 | 66 |
| MOR13871 | 43 | 74 | 105 | 83 | 608 | 68 |
| MOR14535 | 533 | 73 | 231 | 76 | nd | nd |
| MOR14536 | 379 | 83 | 239 | 76 | nd | nd |

To determine whether inhibition of HER3 activity impacted downstream cell signaling, Akt, phosphorylation was also measured in NRG stimulated MCF7 cells and HER2 amplified SK-Br-3/BT474 cells following treatment with anti-HER3 antibodies (see FIG. 6, FIG. 7 and Table 7).

TABLE 7
pAkt (S⁴⁷³) IC₅₀ and extent of inhibition values of anti-HER3 IgG in
SK-Br-3, BT-474 and MCF7 cells.

| | SK-Br-3 | | | | | |
|---|---|---|---|---|---|---|
| | pAkt | | BT-474 pAkt | | MCF7 pAkt | |
| | IC₅₀ | % | IC₅₀ | % | IC₅₀ | % inhi- |
| MOR# | (pM) | inhibition | (pM) | inhibition | (pM) | bition |
| MOR12509 | nd | nd | nd | nd | nd | nd |
| MOR12510 | nd | nd | nd | nd | nd | nd |
| MOR12616 | 88 | 89 | 316 | 78 | 442 | 80 |
| MOR12923 | nd | nd | nd | nd | nd | nd |
| MOR12924 | nd | nd | nd | nd | nd | nd |
| MOR12925 | 93 | 88 | 255 | 80 | 295 | 82 |
| MOR13750 | 1644 | 83 | 17780 | 60 | nd | nd |
| MOR13752 | 734 | 86 | 39100 | 76 | nd | nd |
| MOR13754 | 465 | 84 | nd | 64 | nd | nd |
| MOR13755 | 131 | 91 | 645 | 79 | 468 | 79 |
| MOR13756 | 107 | 90 | 189 | 86 | nd | nd |
| MOR13758 | 110 | 90 | 371 | 78 | nd | nd |
| MOR13759 | 60 | 91 | 274 | 81 | 248 | 76 |
| MOR13761 | 52 | 86 | nd | 83 | nd | nd |
| MOR13762 | 63 | 86 | 291 | 86 | nd | nd |
| MOR13763 | 76 | 84 | 378 | 76 | nd | nd |
| MOR13765 | 113 | 90 | 1178 | 73 | 412 | 71 |
| MOR13766 | 89 | 91 | 1194 | 75 | 296 | 67 |
| MOR13767 | 92 | 89 | 167 | 82 | nd | nd |
| MOR13768 | 105 | 94 | 466 | 81 | 347 | 73 |
| MOR13867 | 120 | 95 | 472 | 84 | nd | nd |
| MOR13868 | 78 | 92 | 537 | 81 | nd | nd |
| MOR13869 | 212 | 91 | 3035 | 79 | nd | nd |
| MOR13870 | 76 | 91 | 424 | 77 | nd | nd |
| MOR13871 | 97 | 93 | 1627 | 83 | nd | nd |
| MOR14535 | nd | nd | nd | nd | nd | nd |
| MOR14536 | nd | nd | nd | nd | nd | nd |

In summary MOR12509, MOR12510, MOR12616, MOR12923, MOR12924, MOR12925, MOR13750, MOR13752, MOR13754, MOR13755, MOR13756, MOR13758, MOR13759, MOR13761, MOR13762, MOR13763, MOR13765, MOR13766, MOR13767, MOR13768, MOR13867, MOR13868, MOR13869, MOR13870, MOR13871, MOR14535 and MOR14536 are each capable of inhibiting cellular HER3 activity in both a ligand-dependent and ligand-independent manner.

(vii) Inhibition of Proliferation

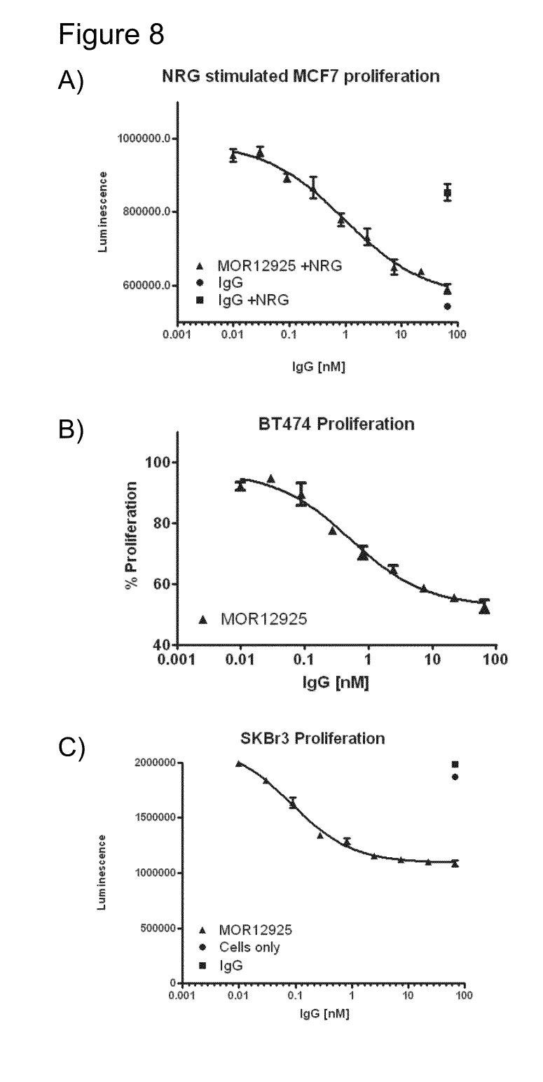

Since MOR12509, MOR12510, MOR12616, MOR12923, MOR12924, MOR12925, MOR13750, MOR13752, MOR13754, MOR13755, MOR13756, MOR13758, MOR13759, MOR13761, MOR13762, MOR13763, MOR13765, MOR13766, MOR13767, MOR13768, MOR13867, MOR13868, MOR13869, MOR13870, MOR13871, MOR14535 and MOR14536 inhibited HER3 activity and downstream signaling they were tested for their ability to block ligand dependent and independent in vitro cell growth (Example data is shown in FIG. 8 and summarized in Table 8). The anti-HER3 antibodies tested were effective inhibitors of cell proliferation confirming their ability to inhibit ligand dependent and independent HER3 driven phenotypes.

TABLE 8
Inhibition of proliferation following treatment with anti-HER3 IgG in
SK-Br-3, BT-474 and MCF7 cells.

| | SK-Br-3 | | BT-474 | | MCF7 | |
|---|---|---|---|---|---|---|
| | IC₅₀ | % | IC₅₀ | % | IC₅₀ | % |
| MOR# | (pM) | inhibition | (pM) | inhibition | (pM) | inhibition |
| MOR12509 | 572 | 45 | nd | nd | 1472 | 24 |
| MOR12510 | 355 | 44 | nd | nd | 483 | 25 |
| MOR12616 | 123 | 48 | 635 | 45 | 415 | 32 |
| MOR12923 | 62 | 42 | nd | nd | 266 | 29 |
| MOR12924 | 533 | 44 | nd | nd | 497 | 23 |
| MOR12925 | 101 | 40 | 522 | 47 | 604 | 47 |
| MOR13750 | 5469 | 34 | 13780 | 21 | nd | nd |
| MOR13752 | 3005 | 40 | 42752 | 22 | nd | nd |
| MOR13754 | 6474 | 40 | 8005 | 17 | nd | nd |
| MOR13755 | 207 | 52 | 738 | 41 | nd | nd |
| MOR13756 | 97 | 50 | 710 | 42 | nd | nd |
| MOR13758 | 133 | 47 | 516 | 38 | nd | nd |
| MOR13759 | 230 | 41 | 6258 | 35 | 151 | 49 |
| MOR13761 | 134 | 48 | 560 | 36 | nd | nd |
| MOR13762 | 53 | 45 | 546 | 37 | nd | nd |
| MOR13763 | 326 | 49 | 671 | 36 | nd | nd |
| MOR13765 | 732 | 46 | 3100 | 27 | nd | nd |
| MOR13766 | 2350 | 45 | 2555 | 37 | nd | 53 |
| MOR13767 | 285 | 48 | 924 | 48 | nd | nd |
| MOR13768 | 198 | 43 | 1211 | 43 | nd | 58 |
| MOR13867 | 310 | 41 | 376 | 42 | 56 | 69 |
| MOR13868 | 251 | 29 | 1719 | 39 | | 58 |
| MOR13869 | 5413 | 24 | 10570 | 28 | | 52 |
| MOR13870 | 1135 | 39 | 539 | 43 | 61 | 48 |
| MOR13871 | 1290 | 32 | 1002 | 43 | nd | 58 |
| MOR14535 | 271 | 66 | nd | nd | nd | nd |
| MOR14536 | 181 | 56 | nd | nd | nd | nd |

(viii) In Vivo Inhibition of Tumor Growth

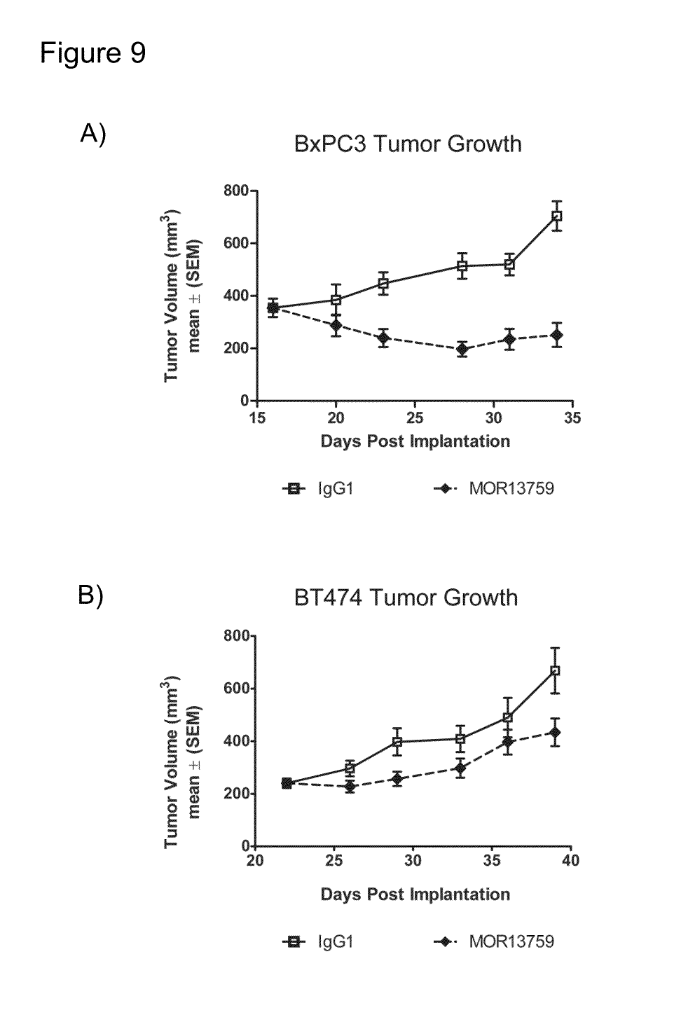

To determine the in vivo activity of the described anti-HER3 antibody, MOR13759 was tested in both BxPC3 and BT-474 tumor models. Repeated MOR13759 treatment yielded a 29.1% regression for the BxPC3 model (FIG. 9A). Treatment of the BT474 model with MOR13759 resulted in 45% inhibition of tumor growth (T/C) (FIG. 9B).

### INCORPORATION BY REFERENCE

All references cited herein, including patents, patent applications, papers, text books, and the like, and the references cited therein, to the extent that they are not already, are hereby incorporated herein by reference in their entirety.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. An isolated antibody or fragment thereof that recognizes an epitope of a HER3 receptor, wherein the epitope comprises amino acid residues 208-328 within domain 2 of the HER3 receptor, wherein the antibody or fragment thereof recognizes at least amino acid residue 268 within domain 2, and wherein the antibody or fragment thereof blocks both ligand-dependent and ligand-independent signal transduction.

2. The isolated antibody or fragment thereof of claim 1, wherein the eptiope is selected from the group consisting of a linear epitope, a non-linear epitope, and a conformational epitope.

3. The isolated antibody or fragment thereof of claim 1, wherein the antibody or fragment thereof binds to an inactive state of the HER3 receptor.

4. The isolated antibody or fragment thereof of claim 1, wherein HER3 ligand binding to the ligand binding site fails to activate HER3 signal transduction.

5. The isolated antibody or fragment thereof of claim 1, wherein a HER3 ligand can concurrently bind to the ligand binding site on the HER3 receptor.

6. The isolated antibody or fragment thereof of claim 5, wherein the HER3 ligand is selected from the group consisting of neuregulin 1 (NRG), neuregulin 2, betacellulin, heparin-binding epidermal growth factor, and epiregulin.

7. The isolated antibody or fragment thereof of claim 1, wherein at least amino acid residue 268 (within domain 2) affects binding in domain 2, thereby blocking antibody or antibody fragment binding.

8. The isolated antibody or fragment thereof wherein the antibody or fragment thereof has a characteristic selected from the group consisting of destabilizing HER3 such that it is susceptale to degradation, accelerating down regulation of cell surface HER3, inhibiting dimerization with other HER receptors, and generating an un-natural HER3 dimer that is susceptible to proteolytic degradation or unable to dimerize with other receptor tyrosine kinases.

9. The isolated antibody or fragment thereof of claim 1, wherein binding of the antibody or fragment thereof to the HER3 receptor in the absence of a HER3 ligand reduces ligand-independent formation of a HER2-HER3 protein complex in a cell which expresses HER2 and HER3.

10. The isolated antibody or fragment thereof of claim 9, wherein the HER3 receptor fails to dimerize with the HER2 receptor to form a HER2-HER3 protein complex.

11. The isolated antibody or fragment thereof of claim 10, wherein the failure to form a HER2-HER3 protein complex prevents activation of signal transduction.

12. The isolated antibody or fragment thereof of claim 9, wherein the antibody or fragment thereof inhibits phosphorylation of HER3 as assessed by a HER3 ligand-independent phosphorylation assay.

13. The isolated antibody or fragment thereof of claim 12, wherein the HER3 ligand-independent phosphorylation assay uses HER2 amplified cells, wherein the HER2 amplified cells are SK-Br-3 cells and BT-474.

14. The isolated antibody or fragment thereof of claim 1, wherein binding of the antibody or fragment thereof to the HER3 receptor in the presence of a HER3 ligand reduces ligand-dependent formation of a HER2-HER3 protein complex in a cell which expresses HER2 and HER3.

15. The isolated antibody or fragment thereof of claim 12, wherein the HER3 receptor fails to dimerize with the HER2 receptor in the presence of a HER3 ligand to form a HER2-HER3 protein complex.

16. The isolated antibody or fragment thereof of claim 13, wherein the failure to form a HER2-HER3 protein complex prevents activation of signal transduction.

17. The isolated antibody or fragment thereof of claim 14, wherein the antibody or fragment thereof inhibits phosphorylation of HER3 as assessed by HER3 ligand-dependent phosphorylation assay.

18. The isolated antibody or fragment thereof of claim 17, wherein the HER3 ligand-dependent phosphorylation assay uses stimulated MCF7 cells in the presence of neuregulin (NRG).

19. The isolated antibody or fragment thereof of claim 1, wherein the antibody is selected from the group consisting of a monoclonal antibody, a polyclonal antibody, a chimeric antibody, a humanized antibody, and a synthetic antibody.

20. An isolated antibody or fragment thereof that recognizes a epitope of a HER3 receptor within domain 2 of the HER3 receptor, wherein the epitope comprises amino acid residues 208-328 within domain 2 of the HER3 receptor, wherein the antibody or fragment thereof recognizes at least amino acid residue 268 within domain 2, and wherein the antibody or fragment thereof has a dissociation (K_{D}) of at least 1×10⁷ M⁻¹, 10⁸ M⁻¹, 10⁹ M⁻¹, 10¹⁰ M⁻¹, 10¹¹ M⁻¹, 10¹² M⁻¹, 10¹³ M⁻¹, and wherein the antibody or fragment thereof blocks both ligand-dependent and ligand-independent signal transduction.

21. The isolated antibody or fragment thereof of claim 20, wherein the antibody or fragment thereof inhibits phosphorylation of HER3 as measured by an in vitro phosphorylation assay selected from the group consisting of phospho-HER3 and phospho-Akt.

22. The isolated antibody or fragment thereof of claim 20, wherein the antibody or fragment thereof binds to the same epitope as an antibody described in Table 1.

23. The isolated antibody or fragment thereof of claim 20, wherein the isolated antibody or fragment thereof cross-competes with an antibody described in Table 1.

24. The isolated antibody or fragment thereof of claim 20, wherein the fragment of an antibody that selected from the group consisting of Fab, F(ab₂)′, F(ab)₂′, scFv, VHH, VH, VL, dAbs.

25. A pharmaceutical composition comprising an antibody or fragment thereof and a pharmaceutically acceptable carrier, wherein the antibody or fragment thereof binds a HER3 receptor comprising amino acid residues 208-328 within domain 2 of the HER3 receptor, wherein the antibody or fragment thereof recognizes at least amino acid residue 268 within domain 2, and wherein the antibody or fragment thereof blocks both ligand-dependent and ligand-independent signal transduction.

26. The pharmaceutical composition of claim 25, further comprising an additional therapeutic agent.

27. The pharmaceutical composition of claim 26, wherein the additional therapeutic agent is selected from the group consisting of an HER1 inhibitor, a HER2 inhibitor, a HER3 inhibitor, a HER4 inhibitor, an mTOR inhibitor and a PI3 Kinase inhibitor.

28. The pharmaceutical composition of claim 27, wherein the additional therapeutic agent is a HER1 inhibitor selected from the group consisting of Matuzumab (EMD72000), Erbitux®/Cetuximab, Vectibix®/Panitumumab, mAb 806, Nimotuzumab, Iressa®/Gefitinib, CI-1033 (PD183805), Lapatinib (GW-572016), Tykerb®/Lapatinib Ditosylate, Tarceva®/Erlotinib HCL (OSI-774), PKI-166, and Tovok®; a HER2 inhibitor selected from the group consisting of Pertuzumab, Trastuzumab, MM-111, neratinib, lapatinib or lapatinib ditosylate/Tykerb®; a HER3 inhibitor selected from the group consisting of, MM-121, MM-111, IB4C3, 2DID12 (U3 Pharma AG), AMG888 (Amgen), AV-203 (Aveo), MEHD7945A (Genentech), MOR10703 (Novartis), and small molecules that inhibit HER3; and a HER4 inhibitor.

29. The pharmaceutical composition of claim 27, wherein the additional therapeutic agent is an mTOR inhibitor selected from the group consisting of Temsirolimus/Torisel®, ridaforolimus/Deforolimus, AP23573, MK8669, everolimus/Affinitor®.

30. The pharmaceutical composition of claim 27, wherein the additional therapeutic agent is a PI3 Kinase inhibitor selected from the group consisting of GDC 0941, BEZ235, BMK120 and BYL719.

31. A method of treating a cancer comprising selecting a subject having an HER3 expressing cancer, administering to the subject an effective amount of a composition comprising an antibody or fragment thereof disclosed in Table 1, wherein the antibody or fragment thereof recognizes an epitope of a HER3 receptor comprising amino acid residues 208-328 within domain 2 of the HER3 receptor, wherein the antibody or fragment thereof recognizes at least amino acid residue 268 within domain 2, and wherein the antibody or fragment thereof blocks both ligand-dependent and ligand-independent signal transduction.

32. The method of claim 31, wherein the subject is a human and the cancer is selected from the group consisting of breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia (BPH), gynacomastica, and endometriosis.

33. The method of claim 31, wherein the cancer is breast cancer.


SEQUENCE LISTING (542 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 5 aa, Homo sapiens
NSWVA

SEQ ID NO: 3 - protein, 17 aa, Homo sapiens
IIYPGNSDTIYSPSFQG

SEQ ID NO: 4 - protein, 17 aa, Homo sapiens
VHIIQPPSAWSYNAMDV

SEQ ID NO: 5 - protein, 7 aa, Homo sapiens
GYSFTNS

SEQ ID NO: 6 - protein, 6 aa, Homo sapiens
YPGNSD

SEQ ID NO: 7 - protein, 17 aa, Homo sapiens
VHIIQPPSAWSYNAMDV

SEQ ID NO: 8 - protein, 11 aa, Homo sapiens
RASQSITHYLN

SEQ ID NO: 9 - protein, 7 aa, Homo sapiens
GASNLQS

SEQ ID NO: 10 - protein, 10 aa, Homo sapiens
QQAFVDPSTT

SEQ ID NO: 11 - protein, 7 aa, Homo sapiens
SQSITHY

SEQ ID NO: 12 - protein, 3 aa, Homo sapiens
GAS

SEQ ID NO: 13 - protein, 7 aa, Homo sapiens
AFVDPST

SEQ ID NO: 14 - protein, 126 aa, Homo sapiens
QVQLVQSGAEVKKPGESLKISCKGSGYSFTNSWVAWVRQMPGKGLEWMGIIYPGNSDTIYSPSFQGQVTISADKSISTAYLQWSSLKASDTAMYYCARVHIIQPPSAWSYNAMDVWGQGTLVTVSS

SEQ ID NO: 15 - protein, 108 aa, Homo sapiens
DIQMTQSPSSLSASVGDRVTITCRASQSITHYLNWYQQKPGKAPKLLIFGASNLQSGVPSRFSGSGSGTDFTLTISSLQPEDFATYYCQQAFVDPSTTFGQGTKVEIK

SEQ ID NO: 16 - DNA, 378 bp, Homo sapiens
caggtgcaattggtgcagagcggtgcggaagtgaaaaaaccgggcgaaagcctgaaaattagctgcaaaggctccggatatagcttcactaactcttgggttgcttgggtgcgccagatgccgggcaaaggtctcgagtggatgggcatcatctacccgggtaacagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 17 - DNA, 324 bp, Homo sapiens
gatatccagatgacccagagcccgagcagcctgagcgccagcgtgggcgatcgcgtgaccattacctgcagagccagccagtctattactcattacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcaggctttcgttgacccgtctactacctttggccagggcacgaaagttgaaattaaa

SEQ ID NO: 18 - protein, 456 aa, Homo sapiens
QVQLVQSGAEVKKPGESLKISCKGSGYSFTNSWVAWVRQMPGKGLEWMGIIYPGNSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 215 aa, Homo sapiens
DIQMTQSPSSLSASVGDRVTITCRASQSITHYLNWYQQKPGKAPKLLIFGASNLQSGVPSRFSGSGSGTDFTLTISSLQPEDFATYYCQQAFVDPSTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 1368 bp, Homo sapiens
caggtgcaattggtgcagagcggtgcggaagtgaaaaaaccgggcgaaagcctgaaaattagctgcaaaggctccggatatagcttcactaactcttgggttgcttgggtgcgccagatgccgggcaaaggtctcgagtggatgggcatcatctacccgggtaacagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - DNA, 645 bp, Homo sapiens
gatatccagatgacccagagcccgagcagcctgagcgccagcgtgggcgatcgcgtgaccattacctgcagagccagccagtctattactcattacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcaggctttcgttgacccgtctactacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 22 - protein, 5 aa, Homo sapiens
NSWVA

SEQ ID NO: 23 - protein, 17 aa, Homo sapiens
IIYPGNSDTIYSPSFQG

SEQ ID NO: 24 - protein, 17 aa, Homo sapiens
VHIIQPPSAWSYNAMDV

SEQ ID NO: 25 - protein, 7 aa, Homo sapiens
GYSFTNS

SEQ ID NO: 26 - protein, 6 aa, Homo sapiens
YPGNSD

SEQ ID NO: 27 - protein, 17 aa, Homo sapiens
VHIIQPPSAWSYNAMDV

SEQ ID NO: 28 - protein, 11 aa, Homo sapiens
RASQSISTYLN

SEQ ID NO: 29 - protein, 7 aa, Homo sapiens
GASNLQS

SEQ ID NO: 30 - protein, 9 aa, Homo sapiens
QQDIDEPWT

SEQ ID NO: 31 - protein, 7 aa, Homo sapiens
SQSISTY

SEQ ID NO: 32 - protein, 3 aa, Homo sapiens
GAS

SEQ ID NO: 33 - protein, 6 aa, Homo sapiens
DIDEPW

SEQ ID NO: 34 - protein, 126 aa, Homo sapiens; identical to SEQ ID NO: 14

SEQ ID NO: 35 - protein, 107 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQDIDEPWTFGQGTKVEIK

SEQ ID NO: 36 - DNA, 378 bp, Homo sapiens; identical to SEQ ID NO: 16

SEQ ID NO: 37 - DNA, 321 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcaggacatcgacgaaccgtggacctttggccagggcacgaaagttgaaattaaa

SEQ ID NO: 38 - protein, 456 aa, Homo sapiens; identical to SEQ ID NO: 18

SEQ ID NO: 39 - protein, 214 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQDIDEPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 1368 bp, Homo sapiens; identical to SEQ ID NO: 20

SEQ ID NO: 41 - DNA, 642 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcaggacatcgacgaaccgtggacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 42 - protein, 5 aa, Homo sapiens
NSWVA

SEQ ID NO: 43 - protein, 17 aa, Homo sapiens
IIYPGNSDTIYSPSFQG

SEQ ID NO: 44 - protein, 17 aa, Homo sapiens
VHIIQPPSAWSYNAMDV

SEQ ID NO: 45 - protein, 7 aa, Homo sapiens
GYSFTNS

SEQ ID NO: 46 - protein, 6 aa, Homo sapiens
YPGNSD

SEQ ID NO: 47 - protein, 17 aa, Homo sapiens
VHIIQPPSAWSYNAMDV

SEQ ID NO: 48 - protein, 11 aa, Homo sapiens
RASQSISTYLN

SEQ ID NO: 49 - protein, 7 aa, Homo sapiens
GASNLQS

SEQ ID NO: 50 - protein, 9 aa, Homo sapiens
QQSITELFT

SEQ ID NO: 51 - protein, 7 aa, Homo sapiens
SQSISTY

SEQ ID NO: 52 - protein, 3 aa, Homo sapiens
GAS

SEQ ID NO: 53 - protein, 6 aa, Homo sapiens
SITELF

SEQ ID NO: 54 - protein, 126 aa, Homo sapiens; identical to SEQ ID NO: 14

SEQ ID NO: 55 - protein, 107 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQSITELFTFGQGTKVEIK

SEQ ID NO: 56 - DNA, 378 bp, Homo sapiens; identical to SEQ ID NO: 16

SEQ ID NO: 57 - DNA, 321 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcagtctatcactgaactgttcacctttggccagggcacgaaagttgaaattaaa

SEQ ID NO: 58 - protein, 456 aa, Homo sapiens; identical to SEQ ID NO: 18

SEQ ID NO: 59 - protein, 214 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQSITELFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 1368 bp, Homo sapiens; identical to SEQ ID NO: 20

SEQ ID NO: 61 - DNA, 642 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcagtctatcactgaactgttcacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 62 - protein, 5 aa, Homo sapiens
NSWVA

SEQ ID NO: 63 - protein, 17 aa, Homo sapiens
IIYPGNSDTIYSPSFQG

SEQ ID NO: 64 - protein, 17 aa, Homo sapiens
VHIIQPPSAWSYNAMDV

SEQ ID NO: 65 - protein, 7 aa, Homo sapiens
GYSFTNS

SEQ ID NO: 66 - protein, 6 aa, Homo sapiens
YPGNSD

SEQ ID NO: 67 - protein, 17 aa, Homo sapiens
VHIIQPPSAWSYNAMDV

SEQ ID NO: 68 - protein, 11 aa, Homo sapiens
RASQSISTYLN

SEQ ID NO: 69 - protein, 7 aa, Homo sapiens
GASNLQS

SEQ ID NO: 70 - protein, 9 aa, Homo sapiens
QQSMYLPFT

SEQ ID NO: 71 - protein, 7 aa, Homo sapiens
SQSISTY

SEQ ID NO: 72 - protein, 3 aa, Homo sapiens
GAS

SEQ ID NO: 73 - protein, 6 aa, Homo sapiens
SMYLPF

SEQ ID NO: 74 - protein, 126 aa, Homo sapiens; identical to SEQ ID NO: 14

SEQ ID NO: 75 - protein, 107 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQSMYLPFTFGQGTKVEIK

SEQ ID NO: 76 - DNA, 378 bp, Homo sapiens; identical to SEQ ID NO: 16

SEQ ID NO: 77 - DNA, 321 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcagtctatgtacctgccgttcacctttggccagggcacgaaagttgaaattaaa

SEQ ID NO: 78 - protein, 456 aa, Homo sapiens; identical to SEQ ID NO: 18

SEQ ID NO: 79 - protein, 214 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQSMYLPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 1368 bp, Homo sapiens; identical to SEQ ID NO: 20

SEQ ID NO: 81 - DNA, 642 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcagtctatgtacctgccgttcacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 82 - protein, 5 aa, Homo sapiens
NSWVA

SEQ ID NO: 83 - protein, 17 aa, Homo sapiens
IIYPGNSDTIYSPSFQG

SEQ ID NO: 84 - protein, 17 aa, Homo sapiens
VHIIQPPSAWSYNAMDV

SEQ ID NO: 85 - protein, 7 aa, Homo sapiens
GYSFTNS

SEQ ID NO: 86 - protein, 6 aa, Homo sapiens
YPGNSD

SEQ ID NO: 87 - protein, 17 aa, Homo sapiens
VHIIQPPSAWSYNAMDV

SEQ ID NO: 88 - protein, 11 aa, Homo sapiens
RASQSISTYLN

SEQ ID NO: 89 - protein, 7 aa, Homo sapiens
GASNLQS

SEQ ID NO: 90 - protein, 9 aa, Homo sapiens
QQSVWEPAT

SEQ ID NO: 91 - protein, 7 aa, Homo sapiens
SQSISTY

SEQ ID NO: 92 - protein, 3 aa, Homo sapiens
GAS

SEQ ID NO: 93 - protein, 6 aa, Homo sapiens
SVWEPA

SEQ ID NO: 94 - protein, 126 aa, Homo sapiens; identical to SEQ ID NO: 14

SEQ ID NO: 95 - protein, 107 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQSVWEPATFGQGTKVEIK

SEQ ID NO: 96 - DNA, 378 bp, Homo sapiens; identical to SEQ ID NO: 16

SEQ ID NO: 97 - DNA, 321 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcagtctgtttgggaaccggctacctttggccagggcacgaaagttgaaattaaa

SEQ ID NO: 98 - protein, 456 aa, Homo sapiens; identical to SEQ ID NO: 18

SEQ ID NO: 99 - protein, 214 aa, Homo sapiens
DIQMTQSPSSLSASVGDRVTITCRASQSISTYLNWYQQKPGKAPKLLIFGASNLQSGVPSRFSGSGSGTDFTLTISSLQPEDFATYYCQQSVWEPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 1368 bp, Homo sapiens; identical to SEQ ID NO: 20

SEQ ID NO: 101 - DNA, 642 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatcttcggtgcttctaacctgcaaagcggcgtgccgagccgctttagcggcagcggatccggcaccgatttcaccctgaccattagctctctgcaaccggaagactttgcgacctattattgccagcagtctgtttgggaaccggctacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 102 - protein, 5 aa, Homo sapiens
NYWIA

SEQ ID NO: 103 - protein, 17 aa, Homo sapiens
IIYPGNSDTIYSPSFQG

SEQ ID NO: 104 - protein, 17 aa, Homo sapiens
VHIIQPPSAWSYNAMDV

SEQ ID NO: 105 - protein, 7 aa, Homo sapiens
GYSFSNY

SEQ ID NO: 106 - protein, 6 aa, Homo sapiens
YPGNSD

SEQ ID NO: 107 - protein, 17 aa, Homo sapiens
VHIIQPPSAWSYNAMDV

SEQ ID NO: 108 - protein, 11 aa, Homo sapiens
RASQSISTYLN

SEQ ID NO: 109 - protein, 7 aa, Homo sapiens
GASNLQS

SEQ ID NO: 110 - protein, 9 aa, Homo sapiens
QQSITELFT

SEQ ID NO: 111 - protein, 7 aa, Homo sapiens
SQSISTY

SEQ ID NO: 112 - protein, 3 aa, Homo sapiens
GAS

SEQ ID NO: 113 - protein, 6 aa, Homo sapiens
SITELF

SEQ ID NO: 114 - protein, 126 aa, Homo sapiens
QVQLVQSGAEVKKPGESLKISCKGSGYSFSNYWIAWVRQMPGKGLEWMGIIYPGNSDTIYSPSFQGQVTISADKSISTAYLQWSSLKASDTAMYYCARVHIIQPPSAWSYNAMDVWGQGTLVTVSS

SEQ ID NO: 115 - protein, 107 aa, Homo sapiens; identical to SEQ ID NO: 55

SEQ ID NO: 116 - DNA, 378 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtaacagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 117 - DNA, 333 bp, Homo sapiens
gatatcgcgctgacccagccggcgagcgtgagcggtagcccgggccagagcattaccattagctgcaccggcaccagcagcgatgtgggcacttacaaccaggtgtcttggtaccagcagcatccgggcaaggcgccgaaactgatgatctacggtgtttctaaacgtccgagcggcgtgagcaaccgttttagcggatccaaaagcggcaacaccgcgagcctgaccattagcggcctgcaagcggaagacgaagcggattattactgccagtctcgtggtgaataccgtccgggttgggtgtttggcggcggcacgaagttaaccgtccta

SEQ ID NO: 118 - protein, 456 aa, Homo sapiens
QVQLVQSGAEVKKPGESLKISCKGSGYSFSNYWIAWVRQMPGKGLEWMGIIYPGNSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 214 aa, Homo sapiens; identical to SEQ ID NO: 59

SEQ ID NO: 120 - DNA, 1368 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtaacagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 121 - DNA, 642 bp, Homo sapiens; identical to SEQ ID NO: 61

SEQ ID NO: 122 - protein, 5 aa, Homo sapiens
SSWVA

SEQ ID NO: 123 - protein, 17 aa, Homo sapiens
IIYPGGSDTIYSPSFQG

SEQ ID NO: 124 - protein, 17 aa, Homo sapiens
VHIIQPPSAWSYNAMDV

SEQ ID NO: 125 - protein, 7 aa, Homo sapiens
GYSFTSS

SEQ ID NO: 126 - protein, 6 aa, Homo sapiens
YPGGSD

SEQ ID NO: 127 - protein, 17 aa, Homo sapiens
VHIIQPPSAWSYNAMDV

SEQ ID NO: 128 - protein, 11 aa, Homo sapiens
RASQSISTYLN

SEQ ID NO: 129 - protein, 7 aa, Homo sapiens
GASNLQS

SEQ ID NO: 130 - protein, 9 aa, Homo sapiens
QQSITELFT

SEQ ID NO: 131 - protein, 7 aa, Homo sapiens
SQSISTY

SEQ ID NO: 132 - protein, 3 aa, Homo sapiens
GAS

SEQ ID NO: 133 - protein, 6 aa, Homo sapiens
SITELF

SEQ ID NO: 134 - protein, 126 aa, Homo sapiens
QVQLVQSGAEVKKPGESLKISCKGSGYSFTSSWVAWVRQMPGKGLEWMGIIYPGGSDTIYSPSFQGQVTISADKSISTAYLQWSSLKASDTAMYYCARVHIIQPPSAWSYNAMDVWGQGTLVTVSS

SEQ ID NO: 135 - protein, 107 aa, Homo sapiens; identical to SEQ ID NO: 55

SEQ ID NO: 136 - DNA, 363 bp, Homo sapiens
caggtgcaattggtgcagagcggtgcggaagtgaaaaaaccgggtgccagcgtgaaagttagctgcaaagcgtccggatataccttcacttcttacgacatccattgggtgcgccaggccccgggccagggcctcgagtggatgggccgtatcgacccgtactctggcaacacgaactacgcgcagaaatttcagggccgggtgaccatgacccgtgataccagcattagcaccgcgtatatggaactgagccgtctgcgtagcgaagatacggccgtgtattattgcgcgcgtggttctttctacactcgtgactcttacttcgatgtttggggccaaggcaccctggtgactgttagctca

SEQ ID NO: 137 - DNA, 333 bp, Homo sapiens; identical to SEQ ID NO: 117

SEQ ID NO: 138 - protein, 456 aa, Homo sapiens
QVQLVQSGAEVKKPGESLKISCKGSGYSFTSSWVAWVRQMPGKGLEWMGIIYPGGSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 214 aa, Homo sapiens; identical to SEQ ID NO: 59

SEQ ID NO: 140 - DNA, 1353 bp, Homo sapiens
caggtgcaattggtgcagagcggtgcggaagtgaaaaaaccgggtgccagcgtgaaagttagctgcaaagcgtccggatataccttcacttcttacgacatccattgggtgcgccaggccccgggccagggcctcgagtggatgggccgtatcgacccgtactctggcaacacgaactacgcgcagaaatttcagggccgggtgaccatgacccgtgataccagcattagcaccgcgtatatggaactgagccgtctgcgtagcgaagatacggccgtgtattattgcgcgcgtggttctttctacactcgtgactcttacttcgatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 141 - DNA, 651 bp, Homo sapiens
gatatcgcgctgacccagccggcgagcgtgagcggtagcccgggccagagcattaccattagctgcaccggcaccagcagcgatgtgggcacttacaaccaggtgtcttggtaccagcagcatccgggcaaggcgccgaaactgatgatctacggtgtttctaaacgtccgagcggcgtgagcaaccgttttagcggatccaaaagcggcaacaccgcgagcctgaccattagcggcctgcaagcggaagacgaagcggattattactgccagtctcgtggtgaataccgtccgggttgggtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 142 - protein, 5 aa, Homo sapiens
NYWVA

SEQ ID NO: 143 - protein, 17 aa, Homo sapiens
IIYPGGSDTIYSPSFQG

SEQ ID NO: 144 - protein, 17 aa, Homo sapiens
VHIIQPPSAWSYNAMDV

SEQ ID NO: 145 - protein, 7 aa, Homo sapiens
GYSFTNY

SEQ ID NO: 146 - protein, 6 aa, Homo sapiens
YPGGSD

SEQ ID NO: 147 - protein, 17 aa, Homo sapiens
VHIIQPPSAWSYNAMDV

SEQ ID NO: 148 - protein, 11 aa, Homo sapiens
RASQSISTYLN

SEQ ID NO: 149 - protein, 7 aa, Homo sapiens
GASNLQS

SEQ ID NO: 150 - protein, 9 aa, Homo sapiens
QQSITELFT

SEQ ID NO: 151 - protein, 7 aa, Homo sapiens
SQSISTY

SEQ ID NO: 152 - protein, 3 aa, Homo sapiens
GAS

SEQ ID NO: 153 - protein, 6 aa, Homo sapiens
SITELF

SEQ ID NO: 154 - protein, 126 aa, Homo sapiens
QVQLVQSGAEVKKPGESLKISCKGSGYSFTNYWVAWVRQMPGKGLEWMGIIYPGGSDTIYSPSFQGQVTISADKSISTAYLQWSSLKASDTAMYYCARVHIIQPPSAWSYNAMDVWGQGTLVTVSS

SEQ ID NO: 155 - protein, 107 aa, Homo sapiens; identical to SEQ ID NO: 55

SEQ ID NO: 156 - DNA, 378 bp, Homo sapiens
caggtgcaattggtgcagagcggtgcggaagtgaaaaaaccgggcgaaagcctgaaaattagctgcaaaggctccggatatagcttcactaactattgggttgcttgggtgcgccagatgccgggcaaaggtctcgagtggatgggcatcatctacccgggtggcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 157 - DNA, 321 bp, Homo sapiens; identical to SEQ ID NO: 57

SEQ ID NO: 158 - protein, 456 aa, Homo sapiens
QVQLVQSGAEVKKPGESLKISCKGSGYSFTNYWVAWVRQMPGKGLEWMGIIYPGGSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 214 aa, Homo sapiens; identical to SEQ ID NO: 59

SEQ ID NO: 160 - DNA, 1368 bp, Homo sapiens
caggtgcaattggtgcagagcggtgcggaagtgaaaaaaccgggcgaaagcctgaaaattagctgcaaaggctccggatatagcttcactaactattgggttgcttgggtgcgccagatgccgggcaaaggtctcgagtggatgggcatcatctacccgggtggcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 161 - DNA, 642 bp, Homo sapiens; identical to SEQ ID NO: 61

SEQ ID NO: 162 - protein, 5 aa, Homo sapiens
SYWVA

SEQ ID NO: 163 - protein, 17 aa, Homo sapiens
IIYPGGSDTIYSPSFQG

SEQ ID NO: 164 - protein, 17 aa, Homo sapiens
VHIIQPPSAWSYNAMDV

SEQ ID NO: 165 - protein, 7 aa, Homo sapiens
GYSFTSY

SEQ ID NO: 166 - protein, 6 aa, Homo sapiens
YPGGSD

SEQ ID NO: 167 - protein, 17 aa, Homo sapiens
VHIIQPPSAWSYNAMDV

SEQ ID NO: 168 - protein, 11 aa, Homo sapiens
RASQSISTYLN

SEQ ID NO: 169 - protein, 7 aa, Homo sapiens
GASNLQS

SEQ ID NO: 170 - protein, 9 aa, Homo sapiens
QQSITELFT

SEQ ID NO: 171 - protein, 7 aa, Homo sapiens
SQSISTY

SEQ ID NO: 172 - protein, 3 aa, Homo sapiens
GAS

SEQ ID NO: 173 - protein, 6 aa, Homo sapiens
SITELF

SEQ ID NO: 174 - protein, 126 aa, Homo sapiens
QVQLVQSGAEVKKPGESLKISCKGSGYSFTSYWVAWVRQMPGKGLEWMGIIYPGGSDTIYSPSFQGQVTISADKSISTAYLQWSSLKASDTAMYYCARVHIIQPPSAWSYNAMDVWGQGTLVTVSS

SEQ ID NO: 175 - protein, 107 aa, Homo sapiens; identical to SEQ ID NO: 55

SEQ ID NO: 176 - DNA, 378 bp, Homo sapiens
caggtgcaattggtgcagagcggtgcggaagtgaaaaaaccgggcgaaagcctgaaaattagctgcaaaggctccggatatagcttcactagctattgggttgcttgggtgcgccagatgccgggcaaaggtctcgagtggatgggcatcatctacccgggtggcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 177 - DNA, 321 bp, Homo sapiens; identical to SEQ ID NO: 57

SEQ ID NO: 178 - protein, 456 aa, Homo sapiens
QVQLVQSGAEVKKPGESLKISCKGSGYSFTSYWVAWVRQMPGKGLEWMGIIYPGGSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 214 aa, Homo sapiens; identical to SEQ ID NO: 59

SEQ ID NO: 180 - DNA, 1368 bp, Homo sapiens
caggtgcaattggtgcagagcggtgcggaagtgaaaaaaccgggcgaaagcctgaaaattagctgcaaaggctccggatatagcttcactagctattgggttgcttgggtgcgccagatgccgggcaaaggtctcgagtggatgggcatcatctacccgggtggcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 181 - DNA, 642 bp, Homo sapiens; identical to SEQ ID NO: 61

SEQ ID NO: 182 - protein, 5 aa, Homo sapiens
TYWVA

SEQ ID NO: 183 - protein, 17 aa, Homo sapiens
IIYPGQSDTIYSPSFQG

SEQ ID NO: 184 - protein, 17 aa, Homo sapiens
VHIIQPPSAWSYNAMDV

SEQ ID NO: 185 - protein, 7 aa, Homo sapiens
GYSFTTY

SEQ ID NO: 186 - protein, 6 aa, Homo sapiens
YPGQSD

SEQ ID NO: 187 - protein, 17 aa, Homo sapiens
VHIIQPPSAWSYNAMDV

SEQ ID NO: 188 - protein, 11 aa, Homo sapiens
RASQSISTYLN

SEQ ID NO: 189 - protein, 7 aa, Homo sapiens
GASNLQS

SEQ ID NO: 190 - protein, 9 aa, Homo sapiens
QQSITELFT

SEQ ID NO: 191 - protein, 7 aa, Homo sapiens
SQSISTY

SEQ ID NO: 192 - protein, 3 aa, Homo sapiens
GAS

SEQ ID NO: 193 - protein, 6 aa, Homo sapiens
SITELF

SEQ ID NO: 194 - protein, 126 aa, Homo sapiens
QVQLVQSGAEVKKPGESLKISCKGSGYSFTTYWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 195 - protein, 107 aa, Homo sapiens; identical to SEQ ID NO: 55

SEQ ID NO: 196 - DNA, 378 bp, Homo sapiens
caggtgcaattggtgcagagcggtgcggaagtgaaaaaaccgggcgaaagcctgaaaattagctgcaaaggctccggatatagcttcactacctattgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 197 - DNA, 321 bp, Homo sapiens; identical to SEQ ID NO: 57

SEQ ID NO: 198 - protein, 456 aa, Homo sapiens
QVQLVQSGAEVKKPGESLKISCKGSGYSFTTYWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 214 aa, Homo sapiens; identical to SEQ ID NO: 59

SEQ ID NO: 200 - DNA, 1368 bp, Homo sapiens
caggtgcaattggtgcagagcggtgcggaagtgaaaaaaccgggcgaaagcctgaaaattagctgcaaaggctccggatatagcttcactacctattgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 201 - DNA, 642 bp, Homo sapiens; identical to SEQ ID NO: 61

SEQ ID NO: 202 - protein, 5 aa, Homo sapiens
LTWVA

SEQ ID NO: 203 - protein, 17 aa, Homo sapiens
IIYPGQSDTIYSPSFQG

SEQ ID NO: 204 - protein, 17 aa, Homo sapiens
VHIIQPPSAWSYNAMDV

SEQ ID NO: 205 - protein, 7 aa, Homo sapiens
GYSFTLT

SEQ ID NO: 206 - protein, 6 aa, Homo sapiens
YPGQSD

SEQ ID NO: 207 - protein, 17 aa, Homo sapiens
VHIIQPPSAWSYNAMDV

SEQ ID NO: 208 - protein, 11 aa, Homo sapiens
RASQSISTYLN

SEQ ID NO: 209 - protein, 7 aa, Homo sapiens
GASNLQS

SEQ ID NO: 210 - protein, 9 aa, Homo sapiens
QQSITELFT

SEQ ID NO: 211 - protein, 7 aa, Homo sapiens
SQSISTY

SEQ ID NO: 212 - protein, 3 aa, Homo sapiens
GAS

SEQ ID NO: 213 - protein, 6 aa, Homo sapiens
SITELF

SEQ ID NO: 214 - protein, 126 aa, Homo sapiens
QVQLVQSGAEVKKPGESLKISCKGSGYSFTLTWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 215 - protein, 107 aa, Homo sapiens; identical to SEQ ID NO: 55

SEQ ID NO: 216 - DNA, 378 bp, Homo sapiens
caggtgcaattggtgcagagcggtgcggaagtgaaaaaaccgggcgaaagcctgaaaattagctgcaaaggctccggatatagcttcactctcacctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 217 - DNA, 321 bp, Homo sapiens; identical to SEQ ID NO: 57

SEQ ID NO: 218 - protein, 456 aa, Homo sapiens
QVQLVQSGAEVKKPGESLKISCKGSGYSFTLTWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 214 aa, Homo sapiens; identical to SEQ ID NO: 59

SEQ ID NO: 220 - DNA, 1368 bp, Homo sapiens
caggtgcaattggtgcagagcggtgcggaagtgaaaaaaccgggcgaaagcctgaaaattagctgcaaaggctccggatatagcttcactctcacctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 221 - DNA, 642 bp, Homo sapiens; identical to SEQ ID NO: 61

SEQ ID NO: 222 - protein, 5 aa, Homo sapiens
LYWVA

SEQ ID NO: 223 - protein, 17 aa, Homo sapiens
IIYPGQSDTIYSPSFQG

SEQ ID NO: 224 - protein, 17 aa, Homo sapiens
VHIIQPPSAWSYNAMDV

SEQ ID NO: 225 - protein, 7 aa, Homo sapiens
GYSFTLY

SEQ ID NO: 226 - protein, 6 aa, Homo sapiens
YPGQSD

SEQ ID NO: 227 - protein, 17 aa, Homo sapiens
VHIIQPPSAWSYNAMDV

SEQ ID NO: 228 - protein, 11 aa, Homo sapiens
RASQSISTYLN

SEQ ID NO: 229 - protein, 7 aa, Homo sapiens
GASNLQS

SEQ ID NO: 230 - protein, 9 aa, Homo sapiens
QQSITELFT

SEQ ID NO: 231 - protein, 7 aa, Homo sapiens
SQSISTY

SEQ ID NO: 232 - protein, 3 aa, Homo sapiens
GAS

SEQ ID NO: 233 - protein, 6 aa, Homo sapiens
SITELF

SEQ ID NO: 234 - protein, 126 aa, Homo sapiens
QVQLVQSGAEVKKPGESLKISCKGSGYSFTLYWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 235 - protein, 107 aa, Homo sapiens; identical to SEQ ID NO: 55

SEQ ID NO: 236 - DNA, 378 bp, Homo sapiens
caggtgcaattggtgcagagcggtgcggaagtgaaaaaaccgggcgaaagcctgaaaattagctgcaaaggctccggatatagcttcactctctactgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 237 - DNA, 321 bp, Homo sapiens; identical to SEQ ID NO: 57

SEQ ID NO: 238 - protein, 456 aa, Homo sapiens
QVQLVQSGAEVKKPGESLKISCKGSGYSFTLYWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 214 aa, Homo sapiens; identical to SEQ ID NO: 59

SEQ ID NO: 240 - DNA, 1368 bp, Homo sapiens
caggtgcaattggtgcagagcggtgcggaagtgaaaaaaccgggcgaaagcctgaaaattagctgcaaaggctccggatatagcttcactctctactgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 241 - DNA, 642 bp, Homo sapiens; identical to SEQ ID NO: 61

SEQ ID NO: 242 - protein, 5 aa, Homo sapiens
TTWVA

SEQ ID NO: 243 - protein, 17 aa, Homo sapiens
IIYPGLSDTIYSPSFQG

SEQ ID NO: 244 - protein, 17 aa, Homo sapiens
VHIIQPPSAWSYNAMDV

SEQ ID NO: 245 - protein, 7 aa, Homo sapiens
GYSFTTT

SEQ ID NO: 246 - protein, 6 aa, Homo sapiens
YPGLSD

SEQ ID NO: 247 - protein, 17 aa, Homo sapiens
VHIIQPPSAWSYNAMDV

SEQ ID NO: 248 - protein, 11 aa, Homo sapiens
RASQSISTYLN

SEQ ID NO: 249 - protein, 7 aa, Homo sapiens
GASNLQS

SEQ ID NO: 250 - protein, 9 aa, Homo sapiens
QQSITELFT

SEQ ID NO: 251 - protein, 7 aa, Homo sapiens
SQSISTY

SEQ ID NO: 252 - protein, 3 aa, Homo sapiens
GAS

SEQ ID NO: 253 - protein, 6 aa, Homo sapiens
SITELF

SEQ ID NO: 254 - protein, 126 aa, Homo sapiens
QVQLVQSGAEVKKPGESLKISCKGSGYSFTTTWVAWVRQMPGKGLEWMGIIYPGLSDTIYSPSFQGQVTISADKSISTAYLQWSSLKASDTAMYYCARVHIIQPPSAWSYNAMDVWGQGTLVTVSS

SEQ ID NO: 255 - protein, 107 aa, Homo sapiens; identical to SEQ ID NO: 55

SEQ ID NO: 256 - DNA, 378 bp, Homo sapiens
caggtgcaattggtgcagagcggtgcggaagtgaaaaaaccgggcgaaagcctgaaaattagctgcaaaggctccggatatagcttcactaccacttgggttgcttgggtgcgccagatgccgggcaaaggtctcgagtggatgggcatcatctacccgggtctgagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 257 - DNA, 321 bp, Homo sapiens; identical to SEQ ID NO: 57

SEQ ID NO: 258 - protein, 456 aa, Homo sapiens
QVQLVQSGAEVKKPGESLKISCKGSGYSFTTTWVAWVRQMPGKGLEWMGIIYPGLSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 214 aa, Homo sapiens; identical to SEQ ID NO: 59

SEQ ID NO: 260 - DNA, 1368 bp, Homo sapiens
caggtgcaattggtgcagagcggtgcggaagtgaaaaaaccgggcgaaagcctgaaaattagctgcaaaggctccggatatagcttcactaccacttgggttgcttgggtgcgccagatgccgggcaaaggtctcgagtggatgggcatcatctacccgggtctgagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 261 - DNA, 642 bp, Homo sapiens; identical to SEQ ID NO: 61

SEQ ID NO: 262 - protein, 5 aa, Homo sapiens
QTWVA

SEQ ID NO: 263 - protein, 17 aa, Homo sapiens
IIYPGQSDTIYSPSFQG

SEQ ID NO: 264 - protein, 17 aa, Homo sapiens
VHIIQPPSAWSYNAMDV

SEQ ID NO: 265 - protein, 7 aa, Homo sapiens
GYSFTQT

SEQ ID NO: 266 - protein, 6 aa, Homo sapiens
YPGQSD

SEQ ID NO: 267 - protein, 17 aa, Homo sapiens
VHIIQPPSAWSYNAMDV

SEQ ID NO: 268 - protein, 11 aa, Homo sapiens
RASQSISTYLN

SEQ ID NO: 269 - protein, 7 aa, Homo sapiens
GASNLQS

SEQ ID NO: 270 - protein, 9 aa, Homo sapiens
QQSITELFT

SEQ ID NO: 271 - protein, 7 aa, Homo sapiens
SQSISTY

SEQ ID NO: 272 - protein, 3 aa, Homo sapiens
GAS

SEQ ID NO: 273 - protein, 6 aa, Homo sapiens
SITELF

SEQ ID NO: 274 - protein, 126 aa, Homo sapiens
QVQLVQSGAEVKKPGESLKISCKGSGYSFTQTWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 275 - protein, 107 aa, Homo sapiens; identical to SEQ ID NO: 55

SEQ ID NO: 276 - DNA, 378 bp, Homo sapiens
caggtgcaattggtgcagagcggtgcggaagtgaaaaaaccgggcgaaagcctgaaaattagctgcaaaggctccggatatagcttcactcagacctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 277 - DNA, 321 bp, Homo sapiens; identical to SEQ ID NO: 57

SEQ ID NO: 278 - protein, 456 aa, Homo sapiens
QVQLVQSGAEVKKPGESLKISCKGSGYSFTQTWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 214 aa, Homo sapiens; identical to SEQ ID NO: 59

SEQ ID NO: 280 - DNA, 1368 bp, Homo sapiens
caggtgcaattggtgcagagcggtgcggaagtgaaaaaaccgggcgaaagcctgaaaattagctgcaaaggctccggatatagcttcactcagacctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 281 - DNA, 642 bp, Homo sapiens; identical to SEQ ID NO: 61

SEQ ID NO: 282 - protein, 5 aa, Homo sapiens
NTWVA

SEQ ID NO: 283 - protein, 17 aa, Homo sapiens
IIYPGQSDTIYSPSFQG

SEQ ID NO: 284 - protein, 17 aa, Homo sapiens
VHIIQPPSAWSYNAMDV

SEQ ID NO: 285 - protein, 7 aa, Homo sapiens
GYSFTNT

SEQ ID NO: 286 - protein, 6 aa, Homo sapiens
YPGQSD

SEQ ID NO: 287 - protein, 17 aa, Homo sapiens
VHIIQPPSAWSYNAMDV

SEQ ID NO: 288 - protein, 11 aa, Homo sapiens
RASQSISTYLN

SEQ ID NO: 289 - protein, 7 aa, Homo sapiens
GASNLQS

SEQ ID NO: 290 - protein, 9 aa, Homo sapiens
QQSITELFT

SEQ ID NO: 291 - protein, 7 aa, Homo sapiens
SQSISTY

SEQ ID NO: 292 - protein, 3 aa, Homo sapiens
GAS

SEQ ID NO: 293 - protein, 6 aa, Homo sapiens
SITELF

SEQ ID NO: 294 - protein, 126 aa, Homo sapiens
QVQLVQSGAEVKKPGESLKISCKGSGYSFTNTWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 295 - protein, 107 aa, Homo sapiens; identical to SEQ ID NO: 55

SEQ ID NO: 296 - DNA, 378 bp, Homo sapiens
caggtgcaattggtgcagagcggtgcggaagtgaaaaaaccgggcgaaagcctgaaaattagctgcaaaggctccggatatagcttcactaacacctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 297 - DNA, 321 bp, Homo sapiens; identical to SEQ ID NO: 57

SEQ ID NO: 298 - protein, 456 aa, Homo sapiens
QVQLVQSGAEVKKPGESLKISCKGSGYSFTNTWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 214 aa, Homo sapiens; identical to SEQ ID NO: 59

SEQ ID NO: 300 - DNA, 1368 bp, Homo sapiens
caggtgcaattggtgcagagcggtgcggaagtgaaaaaaccgggcgaaagcctgaaaattagctgcaaaggctccggatatagcttcactaacacctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 301 - DNA, 642 bp, Homo sapiens; identical to SEQ ID NO: 61

SEQ ID NO: 302 - protein, 5 aa, Homo sapiens
SYWVA

SEQ ID NO: 303 - protein, 17 aa, Homo sapiens
IIYPGQSDTIYSPSFQG

SEQ ID NO: 304 - protein, 17 aa, Homo sapiens
VHIIQPPSAWSYNAMDV

SEQ ID NO: 305 - protein, 7 aa, Homo sapiens
GYSFTSY

SEQ ID NO: 306 - protein, 6 aa, Homo sapiens
YPGQSD

SEQ ID NO: 307 - protein, 17 aa, Homo sapiens
VHIIQPPSAWSYNAMDV

SEQ ID NO: 308 - protein, 11 aa, Homo sapiens
RASQSISTYLN

SEQ ID NO: 309 - protein, 7 aa, Homo sapiens
GASNLQS

SEQ ID NO: 310 - protein, 9 aa, Homo sapiens
QQSITELFT

SEQ ID NO: 311 - protein, 7 aa, Homo sapiens
SQSISTY

SEQ ID NO: 312 - protein, 3 aa, Homo sapiens
GAS

SEQ ID NO: 313 - protein, 6 aa, Homo sapiens
SITELF

SEQ ID NO: 314 - protein, 126 aa, Homo sapiens
QVQLVQSGAEVKKPGESLKISCKGSGYSFTSYWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 315 - protein, 107 aa, Homo sapiens; identical to SEQ ID NO: 55

SEQ ID NO: 316 - DNA, 378 bp, Homo sapiens
caggtgcaattggtgcagagcggtgcggaagtgaaaaaaccgggcgaaagcctgaaaattagctgcaaaggctccggatatagcttcactagctactgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 317 - DNA, 321 bp, Homo sapiens; identical to SEQ ID NO: 57

SEQ ID NO: 318 - protein, 456 aa, Homo sapiens
QVQLVQSGAEVKKPGESLKISCKGSGYSFTSYWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 214 aa, Homo sapiens; identical to SEQ ID NO: 59

SEQ ID NO: 320 - DNA, 1368 bp, Homo sapiens
caggtgcaattggtgcagagcggtgcggaagtgaaaaaaccgggcgaaagcctgaaaattagctgcaaaggctccggatatagcttcactagctactgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 321 - DNA, 642 bp, Homo sapiens; identical to SEQ ID NO: 61

SEQ ID NO: 322 - protein, 5 aa, Homo sapiens
GSWVA

SEQ ID NO: 323 - protein, 17 aa, Homo sapiens
IIYPGTSDTIYSPSFQG

SEQ ID NO: 324 - protein, 17 aa, Homo sapiens
VHIIQPPSAWSYNAMDV

SEQ ID NO: 325 - protein, 7 aa, Homo sapiens
GYSFTGS

SEQ ID NO: 326 - protein, 6 aa, Homo sapiens
YPGTSD

SEQ ID NO: 327 - protein, 17 aa, Homo sapiens
VHIIQPPSAWSYNAMDV

SEQ ID NO: 328 - protein, 11 aa, Homo sapiens
RASQSISTYLN

SEQ ID NO: 329 - protein, 7 aa, Homo sapiens
GASNLQS

SEQ ID NO: 330 - protein, 9 aa, Homo sapiens
QQSITELFT

SEQ ID NO: 331 - protein, 7 aa, Homo sapiens
SQSISTY

SEQ ID NO: 332 - protein, 3 aa, Homo sapiens
GAS

SEQ ID NO: 333 - protein, 6 aa, Homo sapiens
SITELF

SEQ ID NO: 334 - protein, 126 aa, Homo sapiens
QVQLVQSGAEVKKPGESLKISCKGSGYSFTGSWVAWVRQMPGKGLEWMGIIYPGTSDTIYSPSFQGQVTISADKSISTAYLQWSSLKASDTAMYYCARVHIIQPPSAWSYNAMDVWGQGTLVTVSS

SEQ ID NO: 335 - protein, 107 aa, Homo sapiens; identical to SEQ ID NO: 55

SEQ ID NO: 336 - DNA, 378 bp, Homo sapiens
caggtgcaattggtgcagagcggtgcggaagtgaaaaaaccgggcgaaagcctgaaaattagctgcaaaggctccggatatagcttcactggcagctgggttgcttgggtgcgccagatgccgggcaaaggtctcgagtggatgggcatcatctacccgggtaccagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 337 - DNA, 321 bp, Homo sapiens; identical to SEQ ID NO: 57

SEQ ID NO: 338 - protein, 456 aa, Homo sapiens
QVQLVQSGAEVKKPGESLKISCKGSGYSFTGSWVAWVRQMPGKGLEWMGIIYPGTSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 214 aa, Homo sapiens; identical to SEQ ID NO: 59

SEQ ID NO: 340 - DNA, 1368 bp, Homo sapiens
caggtgcaattggtgcagagcggtgcggaagtgaaaaaaccgggcgaaagcctgaaaattagctgcaaaggctccggatatagcttcactggcagctgggttgcttgggtgcgccagatgccgggcaaaggtctcgagtggatgggcatcatctacccgggtaccagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 341 - DNA, 642 bp, Homo sapiens; identical to SEQ ID NO: 61

SEQ ID NO: 342 - protein, 5 aa, Homo sapiens
TTWVA

SEQ ID NO: 343 - protein, 17 aa, Homo sapiens
IIYPGSSDTIYSPSFQG

SEQ ID NO: 344 - protein, 17 aa, Homo sapiens
VHIIQPPSAWSYNAMDV

SEQ ID NO: 345 - protein, 7 aa, Homo sapiens
GYSFTTT

SEQ ID NO: 346 - protein, 6 aa, Homo sapiens
YPGSSD

SEQ ID NO: 347 - protein, 17 aa, Homo sapiens
VHIIQPPSAWSYNAMDV

SEQ ID NO: 348 - protein, 11 aa, Homo sapiens
RASQSISTYLN

SEQ ID NO: 349 - protein, 7 aa, Homo sapiens
GASNLQS

SEQ ID NO: 350 - protein, 9 aa, Homo sapiens
QQSITELFT

SEQ ID NO: 351 - protein, 7 aa, Homo sapiens
SQSISTY

SEQ ID NO: 352 - protein, 3 aa, Homo sapiens
GAS

SEQ ID NO: 353 - protein, 6 aa, Homo sapiens
SITELF

SEQ ID NO: 354 - protein, 126 aa, Homo sapiens
QVQLVQSGAEVKKPGESLKISCKGSGYSFTTTWVAWVRQMPGKGLEWMGIIYPGSSDTIYSPSFQGQVTISADKSISTAYLQWSSLKASDTAMYYCARVHIIQPPSAWSYNAMDVWGQGTLVTVSS

SEQ ID NO: 355 - protein, 107 aa, Homo sapiens; identical to SEQ ID NO: 55

SEQ ID NO: 356 - DNA, 378 bp, Homo sapiens
caggtgcaattggtgcagagcggtgcggaagtgaaaaaaccgggcgaaagcctgaaaattagctgcaaaggctccggatatagcttcactaccacctgggttgcttgggtgcgccagatgccgggcaaaggtctcgagtggatgggcatcatctacccgggtagcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 357 - DNA, 321 bp, Homo sapiens; identical to SEQ ID NO: 57

SEQ ID NO: 358 - protein, 456 aa, Homo sapiens
QVQLVQSGAEVKKPGESLKISCKGSGYSFTTTWVAWVRQMPGKGLEWMGIIYPGSSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 214 aa, Homo sapiens; identical to SEQ ID NO: 59

SEQ ID NO: 360 - DNA, 1368 bp, Homo sapiens
caggtgcaattggtgcagagcggtgcggaagtgaaaaaaccgggcgaaagcctgaaaattagctgcaaaggctccggatatagcttcactaccacctgggttgcttgggtgcgccagatgccgggcaaaggtctcgagtggatgggcatcatctacccgggtagcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 361 - DNA, 642 bp, Homo sapiens; identical to SEQ ID NO: 61

SEQ ID NO: 362 - protein, 5 aa, Homo sapiens
TSWVA

SEQ ID NO: 363 - protein, 17 aa, Homo sapiens
IIYPGQSDTIYSPSFQG

SEQ ID NO: 364 - protein, 17 aa, Homo sapiens
VHIIQPPSAWSYNAMDV

SEQ ID NO: 365 - protein, 7 aa, Homo sapiens
GYSFTTS

SEQ ID NO: 366 - protein, 6 aa, Homo sapiens
YPGQSD

SEQ ID NO: 367 - protein, 17 aa, Homo sapiens
VHIIQPPSAWSYNAMDV

SEQ ID NO: 368 - protein, 11 aa, Homo sapiens
RASQSISTYLN

SEQ ID NO: 369 - protein, 7 aa, Homo sapiens
GASNLQS

SEQ ID NO: 370 - protein, 9 aa, Homo sapiens
QQSITELFT

SEQ ID NO: 371 - protein, 7 aa, Homo sapiens
SQSISTY

SEQ ID NO: 372 - protein, 3 aa, Homo sapiens
GAS

SEQ ID NO: 373 - protein, 6 aa, Homo sapiens
SITELF

SEQ ID NO: 374 - protein, 126 aa, Homo sapiens
QVQLVQSGAEVKKPGESLKISCKGSGYSFTTSWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 375 - protein, 107 aa, Homo sapiens; identical to SEQ ID NO: 55

SEQ ID NO: 376 - DNA, 378 bp, Homo sapiens
caggtgcaattggtgcagagcggtgcggaagtgaaaaaaccgggcgaaagcctgaaaattagctgcaaaggctccggatatagcttcactaccagctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 377 - DNA, 321 bp, Homo sapiens; identical to SEQ ID NO: 57

SEQ ID NO: 378 - protein, 456 aa, Homo sapiens
QVQLVQSGAEVKKPGESLKISCKGSGYSFTTSWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 214 aa, Homo sapiens; identical to SEQ ID NO: 59

SEQ ID NO: 380 - DNA, 1368 bp, Homo sapiens
caggtgcaattggtgcagagcggtgcggaagtgaaaaaaccgggcgaaagcctgaaaattagctgcaaaggctccggatatagcttcactaccagctgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 381 - DNA, 642 bp, Homo sapiens; identical to SEQ ID NO: 61

SEQ ID NO: 382 - protein, 5 aa, Homo sapiens
SSWVA

SEQ ID NO: 383 - protein, 17 aa, Homo sapiens
IIYPGQSDTIYSPSFQG

SEQ ID NO: 384 - protein, 17 aa, Homo sapiens
VHIIQPPSAWSYNAMDV

SEQ ID NO: 385 - protein, 7 aa, Homo sapiens
GYSFTSS

SEQ ID NO: 386 - protein, 6 aa, Homo sapiens
YPGQSD

SEQ ID NO: 387 - protein, 17 aa, Homo sapiens
VHIIQPPSAWSYNAMDV

SEQ ID NO: 388 - protein, 11 aa, Homo sapiens
RASQSISTYLN

SEQ ID NO: 389 - protein, 7 aa, Homo sapiens
GASNLQS

SEQ ID NO: 390 - protein, 9 aa, Homo sapiens
QQSITELFT

SEQ ID NO: 391 - protein, 7 aa, Homo sapiens
SQSISTY

SEQ ID NO: 392 - protein, 3 aa, Homo sapiens
GAS

SEQ ID NO: 393 - protein, 6 aa, Homo sapiens
SITELF

SEQ ID NO: 394 - protein, 126 aa, Homo sapiens
QVQLVQSGAEVKKPGESLKISCKGSGYSFTSSWVAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 395 - protein, 107 aa, Homo sapiens; identical to SEQ ID NO: 55

SEQ ID NO: 396 - DNA, 378 bp, Homo sapiens
caggtgcaattggtgcagagcggtgcggaagtgaaaaaaccgggcgaaagcctgaaaattagctgcaaaggctccggatatagcttcactagctcatgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 397 - DNA, 321 bp, Homo sapiens; identical to SEQ ID NO: 57

SEQ ID NO: 398 - protein, 456 aa, Homo sapiens
QVQLVQSGAEVKKPGESLKISCKGSGYSFTSSWVAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 399 - protein, 214 aa, Homo sapiens; identical to SEQ ID NO: 59

SEQ ID NO: 400 - DNA, 1368 bp, Homo sapiens
caggtgcaattggtgcagagcggtgcggaagtgaaaaaaccgggcgaaagcctgaaaattagctgcaaaggctccggatatagcttcactagctcatgggttgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 401 - DNA, 642 bp, Homo sapiens; identical to SEQ ID NO: 61

SEQ ID NO: 402 - protein, 5 aa, Homo sapiens
NYWIA

SEQ ID NO: 403 - protein, 17 aa, Homo sapiens
IIYPGQSDTIYSPSFQG

SEQ ID NO: 404 - protein, 17 aa, Homo sapiens
VHIIQPPSAWSYNAMDV

SEQ ID NO: 405 - protein, 7 aa, Homo sapiens
GYSFSNY

SEQ ID NO: 406 - protein, 6 aa, Homo sapiens
YPGQSD

SEQ ID NO: 407 - protein, 17 aa, Homo sapiens
VHIIQPPSAWSYNAMDV

SEQ ID NO: 408 - protein, 11 aa, Homo sapiens
RASQSISTYLN

SEQ ID NO: 409 - protein, 7 aa, Homo sapiens
GASNLQS

SEQ ID NO: 410 - protein, 9 aa, Homo sapiens
QQSITELFT

SEQ ID NO: 411 - protein, 7 aa, Homo sapiens
SQSISTY

SEQ ID NO: 412 - protein, 3 aa, Homo sapiens
GAS

SEQ ID NO: 413 - protein, 6 aa, Homo sapiens
SITELF

SEQ ID NO: 414 - protein, 126 aa, Homo sapiens
QVQLVQSGAEVKKPGESLKISCKGSGYSFSNYWIAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 415 - protein, 107 aa, Homo sapiens; identical to SEQ ID NO: 55

SEQ ID NO: 416 - DNA, 378 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 417 - DNA, 321 bp, Homo sapiens; identical to SEQ ID NO: 57

SEQ ID NO: 418 - protein, 456 aa, Homo sapiens
QVQLVQSGAEVKKPGESLKISCKGSGYSFSNYWIAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 419 - protein, 214 aa, Homo sapiens; identical to SEQ ID NO: 59

SEQ ID NO: 420 - DNA, 1368 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 421 - DNA, 642 bp, Homo sapiens; identical to SEQ ID NO: 61

SEQ ID NO: 422 - protein, 5 aa, Homo sapiens
NYWIA

SEQ ID NO: 423 - protein, 17 aa, Homo sapiens
IIYPGLSDTIYSPSFQG

SEQ ID NO: 424 - protein, 17 aa, Homo sapiens
VHIIQPPSAWSYNAMDV

SEQ ID NO: 425 - protein, 7 aa, Homo sapiens
GYSFSNY

SEQ ID NO: 426 - protein, 6 aa, Homo sapiens
YPGLSD

SEQ ID NO: 427 - protein, 17 aa, Homo sapiens
VHIIQPPSAWSYNAMDV

SEQ ID NO: 428 - protein, 11 aa, Homo sapiens
RASQSISTYLN

SEQ ID NO: 429 - protein, 7 aa, Homo sapiens
GASNLQS

SEQ ID NO: 430 - protein, 9 aa, Homo sapiens
QQSITELFT

SEQ ID NO: 431 - protein, 7 aa, Homo sapiens
SQSISTY

SEQ ID NO: 432 - protein, 3 aa, Homo sapiens
GAS

SEQ ID NO: 433 - protein, 6 aa, Homo sapiens
SITELF

SEQ ID NO: 434 - protein, 126 aa, Homo sapiens
QVQLVQSGAEVKKPGESLKISCKGSGYSFSNYWIAWVRQMPGKGLEWMGIIYPGLSDTIYSPSFQGQVTISADKSISTAYLQWSSLKASDTAMYYCARVHIIQPPSAWSYNAMDVWGQGTLVTVSS

SEQ ID NO: 435 - protein, 107 aa, Homo sapiens; identical to SEQ ID NO: 55

SEQ ID NO: 436 - DNA, 378 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtctgagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 437 - DNA, 321 bp, Homo sapiens; identical to SEQ ID NO: 57

SEQ ID NO: 438 - protein, 456 aa, Homo sapiens
QVQLVQSGAEVKKPGESLKISCKGSGYSFSNYWIAWVRQMPGKGLEWMGIIYPGLSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 439 - protein, 214 aa, Homo sapiens; identical to SEQ ID NO: 59

SEQ ID NO: 440 - DNA, 1368 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtctgagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 441 - DNA, 642 bp, Homo sapiens; identical to SEQ ID NO: 61

SEQ ID NO: 442 - protein, 5 aa, Homo sapiens
NYWIA

SEQ ID NO: 443 - protein, 17 aa, Homo sapiens
IIYPGGSDTIYSPSFQG

SEQ ID NO: 444 - protein, 17 aa, Homo sapiens
VHIIQPPSAWSYNAMDV

SEQ ID NO: 445 - protein, 7 aa, Homo sapiens
GYSFSNY

SEQ ID NO: 446 - protein, 6 aa, Homo sapiens
YPGGSD

SEQ ID NO: 447 - protein, 17 aa, Homo sapiens
VHIIQPPSAWSYNAMDV

SEQ ID NO: 448 - protein, 11 aa, Homo sapiens
RASQSISTYLN

SEQ ID NO: 449 - protein, 7 aa, Homo sapiens
GASNLQS

SEQ ID NO: 450 - protein, 9 aa, Homo sapiens
QQSITELFT

SEQ ID NO: 451 - protein, 7 aa, Homo sapiens
SQSISTY

SEQ ID NO: 452 - protein, 3 aa, Homo sapiens
GAS

SEQ ID NO: 453 - protein, 6 aa, Homo sapiens
SITELF

SEQ ID NO: 454 - protein, 126 aa, Homo sapiens
QVQLVQSGAEVKKPGESLKISCKGSGYSFSNYWIAWVRQMPGKGLEWMGIIYPGGSDTIYSPSFQGQVTISADKSISTAYLQWSSLKASDTAMYYCARVHIIQPPSAWSYNAMDVWGQGTLVTVSS

SEQ ID NO: 455 - protein, 107 aa, Homo sapiens; identical to SEQ ID NO: 55

SEQ ID NO: 456 - DNA, 378 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtggcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 457 - DNA, 321 bp, Homo sapiens; identical to SEQ ID NO: 57

SEQ ID NO: 458 - protein, 456 aa, Homo sapiens
QVQLVQSGAEVKKPGESLKISCKGSGYSFSNYWIAWVRQMPGKGLEWMGIIYPGGSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 459 - protein, 214 aa, Homo sapiens; identical to SEQ ID NO: 59

SEQ ID NO: 460 - DNA, 1368 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtggcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 461 - DNA, 642 bp, Homo sapiens; identical to SEQ ID NO: 61

SEQ ID NO: 462 - protein, 5 aa, Homo sapiens
NYWIA

SEQ ID NO: 463 - protein, 17 aa, Homo sapiens
IIYPGTSDTIYSPSFQG

SEQ ID NO: 464 - protein, 17 aa, Homo sapiens
VHIIQPPSAWSYNAMDV

SEQ ID NO: 465 - protein, 7 aa, Homo sapiens
GYSFSNY

SEQ ID NO: 466 - protein, 6 aa, Homo sapiens
YPGTSD

SEQ ID NO: 467 - protein, 17 aa, Homo sapiens
VHIIQPPSAWSYNAMDV

SEQ ID NO: 468 - protein, 11 aa, Homo sapiens
RASQSISTYLN

SEQ ID NO: 469 - protein, 7 aa, Homo sapiens
GASNLQS

SEQ ID NO: 470 - protein, 9 aa, Homo sapiens
QQSITELFT

SEQ ID NO: 471 - protein, 7 aa, Homo sapiens
SQSISTY

SEQ ID NO: 472 - protein, 3 aa, Homo sapiens
GAS

SEQ ID NO: 473 - protein, 6 aa, Homo sapiens
SITELF

SEQ ID NO: 474 - protein, 126 aa, Homo sapiens
QVQLVQSGAEVKKPGESLKISCKGSGYSFSNYWIAWVRQMPGKGLEWMGIIYPGTSDTIYSPSFQGQVTISADKSISTAYLQWSSLKASDTAMYYCARVHIIQPPSAWSYNAMDVWGQGTLVTVSS

SEQ ID NO: 475 - protein, 107 aa, Homo sapiens; identical to SEQ ID NO: 55

SEQ ID NO: 476 - DNA, 378 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtaccagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 477 - DNA, 321 bp, Homo sapiens; identical to SEQ ID NO: 57

SEQ ID NO: 478 - protein, 456 aa, Homo sapiens
QVQLVQSGAEVKKPGESLKISCKGSGYSFSNYWIAWVRQMPGKGLEWMGIIYPGTSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 479 - protein, 214 aa, Homo sapiens; identical to SEQ ID NO: 59

SEQ ID NO: 480 - DNA, 1368 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtaccagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 481 - DNA, 642 bp, Homo sapiens; identical to SEQ ID NO: 61

SEQ ID NO: 482 - protein, 5 aa, Homo sapiens
NYWIA

SEQ ID NO: 483 - protein, 17 aa, Homo sapiens
IIYPGSSDTIYSPSFQG

SEQ ID NO: 484 - protein, 17 aa, Homo sapiens
VHIIQPPSAWSYNAMDV

SEQ ID NO: 485 - protein, 7 aa, Homo sapiens
GYSFSNY

SEQ ID NO: 486 - protein, 6 aa, Homo sapiens
YPGSSD

SEQ ID NO: 487 - protein, 17 aa, Homo sapiens
VHIIQPPSAWSYNAMDV

SEQ ID NO: 488 - protein, 11 aa, Homo sapiens
RASQSISTYLN

SEQ ID NO: 489 - protein, 7 aa, Homo sapiens
GASNLQS

SEQ ID NO: 490 - protein, 9 aa, Homo sapiens
QQSITELFT

SEQ ID NO: 491 - protein, 7 aa, Homo sapiens
SQSISTY

SEQ ID NO: 492 - protein, 3 aa, Homo sapiens
GAS

SEQ ID NO: 493 - protein, 6 aa, Homo sapiens
SITELF

SEQ ID NO: 494 - protein, 126 aa, Homo sapiens
QVQLVQSGAEVKKPGESLKISCKGSGYSFSNYWIAWVRQMPGKGLEWMGIIYPGSSDTIYSPSFQGQVTISADKSISTAYLQWSSLKASDTAMYYCARVHIIQPPSAWSYNAMDVWGQGTLVTVSS

SEQ ID NO: 495 - protein, 107 aa, Homo sapiens; identical to SEQ ID NO: 55

SEQ ID NO: 496 - DNA, 378 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtagcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 497 - DNA, 321 bp, Homo sapiens; identical to SEQ ID NO: 57

SEQ ID NO: 498 - protein, 456 aa, Homo sapiens
QVQLVQSGAEVKKPGESLKISCKGSGYSFSNYWIAWVRQMPGKGLEWMGIIYPGSSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 499 - protein, 214 aa, Homo sapiens; identical to SEQ ID NO: 59

SEQ ID NO: 500 - DNA, 1368 bp, Homo sapiens
caggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtagcagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 501 - DNA, 642 bp, Homo sapiens; identical to SEQ ID NO: 61

SEQ ID NO: 502 - protein, 5 aa, Homo sapiens
TTWVA

SEQ ID NO: 503 - protein, 17 aa, Homo sapiens
IIYPGLSDTIYSPSFQG

SEQ ID NO: 504 - protein, 17 aa, Homo sapiens
VHIIQPPSAWSYNAMDV

SEQ ID NO: 505 - protein, 7 aa, Homo sapiens
GYSFTTT

SEQ ID NO: 506 - protein, 6 aa, Homo sapiens
YPGLSD

SEQ ID NO: 507 - protein, 17 aa, Homo sapiens
VHIIQPPSAWSYNAMDV

SEQ ID NO: 508 - protein, 11 aa, Homo sapiens
RASQSISTYLN

SEQ ID NO: 509 - protein, 7 aa, Homo sapiens
GASNLQS

SEQ ID NO: 510 - protein, 9 aa, Homo sapiens
QQSITELFT

SEQ ID NO: 511 - protein, 7 aa, Homo sapiens
SQSISTY

SEQ ID NO: 512 - protein, 3 aa, Homo sapiens
GAS

SEQ ID NO: 513 - protein, 6 aa, Homo sapiens
SITELF

SEQ ID NO: 514 - protein, 126 aa, Homo sapiens
EVQLVQSGAEVKKPGESLKISCKGSGYSFTTTWVAWVRQMPGKGLEWMGIIYPGLSDTIYSPSFQGQVTISADKSISTAYLQWSSLKASDTAMYYCARVHIIQPPSAWSYNAMDVWGQGTLVTVSS

SEQ ID NO: 515 - protein, 107 aa, Homo sapiens
DIQMTQSPSSLSASVGDRVTITCRASQSISTYLNWYQQKPGKAPKLLIYGASNLQSGVPSRFSGSGSGTDFTLTISSLQPEDFATYYCQQSITELFTFGQGTKVEIK

SEQ ID NO: 516 - DNA, 378 bp, Homo sapiens
gaggtgcaattggtgcagagcggtgcggaagtgaaaaaaccgggcgaaagcctgaaaattagctgcaaaggctccggatatagcttcactaccacttgggttgcttgggtgcgccagatgccgggcaaaggtctcgagtggatgggcatcatctacccgggtctgagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 517 - DNA, 321 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatctacggtgcttctaacctgcaaagcggcgtgccgagccgctttagcggcagcggatccggcaccgatttcaccctgaccattagctctctgcaaccggaagactttgcgacctattattgccagcagtctatcactgaactgttcacctttggccagggcacgaaagttgaaattaaa

SEQ ID NO: 518 - protein, 456 aa, Homo sapiens
EVQLVQSGAEVKKPGESLKISCKGSGYSFTTTWVAWVRQMPGKGLEWMGIIYPGLSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 519 - protein, 214 aa, Homo sapiens
DIQMTQSPSSLSASVGDRVTITCRASQSISTYLNWYQQKPGKAPKLLIYGASNLQSGVPSRFSGSGSGTDFTLTISSLQPEDFATYYCQQSITELFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 520 - DNA, 1368 bp, Homo sapiens
gaggtgcaattggtgcagagcggtgcggaagtgaaaaaaccgggcgaaagcctgaaaattagctgcaaaggctccggatatagcttcactaccacttgggttgcttgggtgcgccagatgccgggcaaaggtctcgagtggatgggcatcatctacccgggtctgagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 521 - DNA, 642 bp, Homo sapiens
gatatccagatgacccagagcccgagcagcctgagcgccagcgtgggcgatcgcgtgaccattacctgcagagccagccagtctatttctacttacctgaactggtaccagcagaaaccgggcaaagcgccgaaactattaatctacggtgcttctaacctgcaaagcggcgtgccgagccgctttagcggcagcggatccggcaccgatttcaccctgaccattagctctctgcaaccggaagactttgcgacctattattgccagcagtctatcactgaactgttcacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 522 - protein, 5 aa, Homo sapiens
NYWIA

SEQ ID NO: 523 - protein, 17 aa, Homo sapiens
IIYPGQSDTIYSPSFQG

SEQ ID NO: 524 - protein, 17 aa, Homo sapiens
VHIIQPPSAWSYNAMDV

SEQ ID NO: 525 - protein, 7 aa, Homo sapiens
GYSFSNY

SEQ ID NO: 526 - protein, 6 aa, Homo sapiens
YPGQSD

SEQ ID NO: 527 - protein, 17 aa, Homo sapiens
VHIIQPPSAWSYNAMDV

SEQ ID NO: 528 - protein, 11 aa, Homo sapiens
RASQSISTYLN

SEQ ID NO: 529 - protein, 7 aa, Homo sapiens
GASNLQS

SEQ ID NO: 530 - protein, 9 aa, Homo sapiens
QQSITELFT

SEQ ID NO: 531 - protein, 7 aa, Homo sapiens
SQSISTY

SEQ ID NO: 532 - protein, 3 aa, Homo sapiens
GAS

SEQ ID NO: 533 - protein, 6 aa, Homo sapiens
SITELF

SEQ ID NO: 534 - protein, 126 aa, Homo sapiens
EVQLVQSGAEVKKPGESLKISCKGSGYSFSNYWIAWVRQMPGKGLEWMGIIYPGQSDTIYSPSFQGQVTISADKSISTAYLQWSSLKASDTAMYYCARVHIIQPPSAWSYNAMDVWGQGTLVTVSS

SEQ ID NO: 535 - protein, 107 aa, Homo sapiens; identical to SEQ ID NO: 515

SEQ ID NO: 536 - DNA, 378 bp, Homo sapiens
gaggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctca

SEQ ID NO: 537 - DNA, 321 bp, Homo sapiens; identical to SEQ ID NO: 517

SEQ ID NO: 538 - protein, 456 aa, Homo sapiens
EVQLVQSGAEVKKPGESLKISCKGSGYSFSNYWIAWVRQMPGKGLEWMGIIYPGQSDTIYSPSFQGQVTISADKSISTAYLQWSSLKASDTAMYYCARVHIIQPPSAWSYNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 539 - protein, 214 aa, Homo sapiens; identical to SEQ ID NO: 519

SEQ ID NO: 540 - DNA, 1368 bp, Homo sapiens
gaggtgcaattggtgcagagcggtgcggaagtgaaaaaaccgggcgaaagcctgaaaattagctgcaaaggctccggatatagcttctctaactactggatcgcttgggtgcgccagatgccgggcaaaggtctcgagtggatgggcatcatctacccgggtcaaagcgacaccatctatagcccgagctttcagggccaggtgaccattagcgcggataaaagcatcagcaccgcgtatctgcaatggagcagcctgaaagcgagcgataccgcgatgtattattgcgcgcgtgttcatatcatccagccgccgtctgcttggtcttacaacgctatggatgtttggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 541 - DNA, 642 bp, Homo sapiens; identical to SEQ ID NO: 521

SEQ ID NO: 542 - protein, 6 aa, Artificial Sequence
  Description of Artificial Sequence Synthetic 6xHis tag
HHHHHH